(19) United States
(12) Patent Application Publication
(10) Pub. No.: US 2026/0028321 A1
(43) Pub. Date: Jan. 29, 2026

(54) 1,3,4-OXADIAZOLE DERIVATIVE COMPOUNDS AS HISTONE DEACETYLASE 6 INHIBITOR, AND USES THEREOF

(71) Applicant: CHONG KUN DANG PHARMACEUTICAL CORP., Seoul (KR)

(72) Inventors: Chang Sik LEE, Yongin-si, Gyeonggi-do (KR); Jung Taek OH, Yongin-si, Gyeonggi-do (KR); Hyeseung SONG, Yongin-si, Gyeonggi-do (KR); Hyunjin Michael KIM, Yongin-si, Gyeonggi-do (KR)

(21) Appl. No.: 18/854,489

(22) PCT Filed: Apr. 6, 2023
(86) PCT No.: PCT/KR2023/004684

§ 371 Date: Oct. 4, 2024

(30) Foreign Application Priority Data
Apr. 7, 2022 (KR) 10-2022-0043429

(51) Int. Cl. C07D 271/10; A61K 31/4245; A61K 31/496; A61K 31/4995; A61K 31/5377; A61K 31/541; C07D 413/12; C07D 413/14; C07D 487/08; C07D 487/10

(52) U.S. Cl. CPC C07D 271/10; A61K 31/4245; A61K 31/496; A61K 31/4995; A61K 31/5377; A61K 31/541; C07D 413/12; C07D 413/14; C07D 487/08; C07D 487/10

(57) ABSTRACT

The present invention relates to a novel compound having histone deacetylase 6 (HDAC6) inhibitory activity, a method for preparing the same, and uses thereof. The novel compound according to the present invention, a stereoisomer thereof or a pharmaceutically acceptable salt thereof has HDAC6 inhibitory activity, and is effective for preventing or treating HDAC6-mediated diseases including cancer, inflammatory diseases, autoimmune diseases, neurological or neurodegenerative diseases.

### TECHNICAL FIELD

The present invention relates to a 1,3,4-oxadiazole derivative compound having histone deacetylase 6 (HDAC6) inhibitory activity, a method for preparing the same, and uses thereof.

### BACKGROUND ART

Post-translational modifications such as acetylation in cells are very important regulatory modules at the center of biological processes and are strictly controlled by a number of enzymes. Histones are core proteins constituting chromatin, and help DNA condensation by acting as an axis around which DNA is wound. In addition, the balance between acetylation and deacetylation of histones plays a critical role in gene expression.

Histone deacetylases (HDACs) are enzymes that remove acetyl groups of lysine residues of histone proteins constituting chromatin, and are known to be related to gene silencing and to induce cell cycle arrest, angiogenesis inhibition, immunoregulation, cell death, and the like (Hassig et al., Curr. Opin. Chem. Biol. 1997, 1, 300-308). Further, inhibition of HDAC enzyme function has been reported to reduce the activity of cancer cell survival-related factors in vivo and activate cancer cell death-related factors, thereby inducing self-death of cancer cells (Warrell et al, J. Natl. Cancer Inst. 1998, 90, 1621-1625).

In humans, 18 HDACs enzymes are known, and are classified into four classes depending on homology with yeast HDACs. Here, the 11 HDACs using zinc as a cofactor may be divided into 3 groups of Class I (HDAC1, 2, 3 and 8), Class II (IIa: HDAC4, 5, 7 and 9; IIb: HDAC6 and 10), and Class IV (HDAC11). Additionally, 7 HDACs of Class III (SIRT 1-7) use NAD⁺ as a cofactor instead of zinc (Bolden et al., Nat. Rev. Drug. Discov. 2006, 5(9), 769-784).

Even though various HDAC inhibitors are in the preclinical or clinical development stage, up to date, only non-selective HDAC inhibitors are known as anticancer agents, wherein vorinostat (SAHA) and romidepsin (FK228) have been approved for the treatment of cutaneous T-cell lymphoma, and panobinostat (LBH-589) has been approved for the treatment of multiple myeloma. However, non-selective HDACs inhibitors are generally known to cause side effects such as fatigue, nausea, and the like, at high doses (Piekarz et al., Pharmaceuticals 2010, 3, 2751-2767). These side effects have been reported to be caused by inhibition of class I HDACs, and due to these side effects, non-selective HDACs inhibitors have been limited in drug development in areas other than anticancer drugs (Witt et al., Cancer Letters 277 (2009) 8.21).

Meanwhile, it has been reported that selective class II HDAC inhibition may not exhibit the toxicity seen in class I HDAC inhibition, and the development of selective HDAC inhibitors could solve side effects such as toxicity caused by non-selective HDAC inhibition. Thus, the selective HDAC inhibitors have the potential to be developed as effective treatments for various diseases (Matthias et al., Mol. Cell. Biol. 2008, 28, 1688-1701).

HDAC6, which is one of Class IIb HDACs, is mainly present in the cytoplasm and is known to include tubulin protein to be involved in deacetylation of a number of nonhistone substrates (HSP90, cortactin, and the like) (Yao et al., Mol. Cell 2005, 18, 601-607). HDAC6 may have two catalytic domains, and the C-terminal of zinc finger domain may bind to ubiquitinated proteins. HDAC6 is known to play an important role in various diseases such as cancer, inflammatory diseases, autoimmune diseases, neurological diseases, and neurodegenerative disorders, and the like, since it has a number of non-histone proteins as substrates (Santo et al., Blood 2012 119: 2579-258; Vishwakarma et al., International Immunopharmacology 2013, 16, 72-78; Hu et al., J. Neurol. Sci. 2011, 304, 1-8).

Common structural features of various HDAC inhibitors are composed of a cap group, a linker group, and a zinc binding group (ZBG), as shown in the structure of vorinostat below. A number of researchers have conducted studies on the inhibitory activity and selectivity to the enzyme through structural modifications of the cap group and linker group. The zinc-binding group is known to play a more important role in enzyme inhibitory activity and selectivity (Wiest et al., J. Org. Chem. 2013 78: 5051-5065; Methot et al., Bioorg. Med. Chem. Lett. 2008, 18, 973-978).

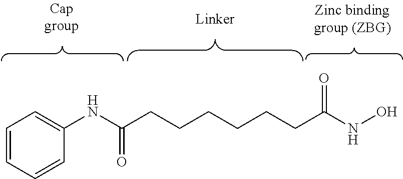 [SMILES: O=C(CCCCCCC(=O)Nc1ccccc1)NO]

Most of the zinc-binding groups are hydroxamic acid or benzamide, wherein the hydroxamic acid derivative show strong HDAC inhibitory effects, but have problems such as low bioavailability and serious off-target activity. The benzamide contains aniline, and thus also has a problem of generating toxic metabolites in vivo (Woster et al., Med. Chem. Commun. 2015, online publication).

Therefore, for the treatment of cancer, inflammatory diseases, autoimmune diseases, neurological diseases, and neurodegenerative disorders, and the like, there is a need to develop a selective HDAC6 inhibitor having a zinc-binding group with no side effects and improved bioavailability, unlike nonselective inhibitors with side effects.

### DISCLOSURE OF INVENTION

### Technical Problem

An object of the present invention is to provide a 1,3,4-oxadiazole derivative compound having selective histone deacetylase 6 (HDAC6) inhibitory activity, a stereoisomer thereof or a pharmaceutically acceptable salt thereof.

Another object of the present invention is to provide a pharmaceutical composition comprising a 1,3,4-oxadiazole derivative compound having selective HDAC6 inhibitory activity, a stereoisomer thereof or a pharmaceutically acceptable salt thereof.

Still another object of the present invention is to provide a method for preparing the same.

Still another object of the present invention is to provide a pharmaceutical composition comprising the compounds for preventing or treating HDAC6 activity-related diseases including infectious diseases; neoplasm; endocrine, nutritional and metabolic diseases; mental and behavioral disorders; neurological diseases; eye and ocular adnexal disease; circulatory diseases; respiratory diseases; digestive diseases; skin and subcutaneous tissue diseases; musculoskeletal and connective tissue diseases; or congenital malformations, alterations, and chromosomal abnormalities.

Still another object of the present invention is to provide uses of the compounds for the preparation of a medicament for preventing or treating HDAC6 activity-related diseases.

Still another object of the present invention is to provide a method for treating HDAC6 activity-related diseases comprising: administering a therapeutically effective amount of a pharmaceutical composition containing the compounds as described above.

### Solution to Problem

The present inventors discovered a 1,3,4-oxadiazole derivative compound having histone deacetylase 6 (HDAC6) inhibitory activity and used the 1,3,4-oxadiazole derivative compound to inhibit or treat HDAC6 activity-related diseases, thereby completing the present invention.

### 1,3,4-Oxadiazole Derivative Compound

In one general aspect, the present invention provides a 1,3,4-oxadiazole derivative compound represented by the following Chemical Formula I, a stereoisomer thereof or a pharmaceutically acceptable salt thereof:

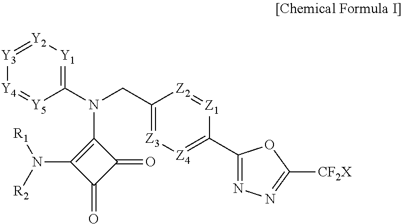

in the Chemical Formula I above,
R₁ and R₂ are each independently —(C₁-C₄alkyl), or R₁ and R₂ are linked to form a heterocycloalkyl together with the N atom, wherein at least one H of the heterocycloalkyl ring may each independently be substituted with —(C₁-C₄alkyl), —(C₁-C₄aminoalkyl), —(C₁-C₄hydroxyalkyl), —(C₁-C₄haloalkyl), -halo, or heterocycloalkyl;
X is —H or —F;
Y₁ to Y₅ are each independently N or CR₃, wherein Y₁ to Y₅ may not be 3 or more N at the same time;
R₃ is —H, —(C₁-C₄alkyl), —(C₁-C₄aminoalkyl), —(C₁-C₄hydroxyalkyl), —(C₁-C₄haloalkyl), -halo, aryl or heteroaryl, wherein at least one —H of the aryl or heteroaryl ring may each independently be substituted with —(C₁-C₄alkyl), —(C₁-C₄aminoalkyl), —(C₁-C₄ hydroxyalkyl), —(C₁-C₄haloalkyl), —OH, —O(C₁-C₄alkyl), or -halo;
Z₁ to Z₄ are each independently N or CR₄, wherein Z₁ to Z₄ may not be 3 or more N at the same time; and
R₄ is —H, —(C₁-C₄alkyl), —(C₁-C₄aminoalkyl), —(C₁-C₄hydroxyalkyl), —(C₁-C₄haloalkyl), -halo, aryl or heteroaryl, wherein at least one —H of the aryl or heteroaryl ring may each independently be substituted with —(C₁-C₄alkyl), —(C₁-C₄aminoalkyl), —(C₁-C₄ hydroxyalkyl), —(C₁-C₄haloalkyl), —OH, —O(C₁-C₄alkyl), or -halo.

According to an embodiment of the present invention, the compound represented by Chemical Formula I, a stereoisomer thereof, or a pharmaceutically acceptable salt thereof may be in the following ranges:
R₁ and R₂ are linked to form a heterocycloalkyl together with the N atom, wherein at least one H of the heterocycloalkyl ring may each independently be substituted with —(C₁-C₄alkyl) or heterocycloalkyl;
X is —H or —F;
Y₁ to Y₅ are each independently CR₃;
R₃ is —H, -halo or heteroaryl, wherein at least one —H of the heteroaryl ring may each independently be substituted with —(C₁-C₄alkyl), —(C₁-C₄aminoalkyl), —(C₁-C₄ hydroxyalkyl), —(C₁-C₄haloalkyl), —OH, —O(C₁-C₄alkyl), or -halo;
Z₁ to Z₄ are each independently N or CR₄, wherein Z₁ to Z₄ may not be 3 or more N at the same time; and
R₄ is —H or -halo.

According to an embodiment of the present invention, the compound represented by Chemical Formula I, a stereoisomer thereof, or a pharmaceutically acceptable salt thereof may be in the following ranges:
R₁ and R₂ are linked to form a 4-12 membered heterocycloalkyl together with the N atom, wherein at least one H of the heterocycloalkyl ring may each independently be substituted with —(C₁-C₄alkyl), —(C₁-C₄aminoalkyl), —(C₁-C₄hydroxyalkyl), —(C₁-C₄ haloalkyl), -halo, or 3-6 membered heterocycloalkyl.

According to an embodiment of the present invention, the compound represented by Chemical Formula I, a stereoisomer thereof, or a pharmaceutically acceptable salt thereof may be in the following ranges:
R₁ and R₂ are linked to form

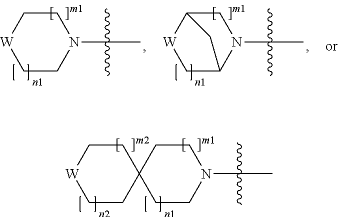

together with the N atom, wherein at least one H of the

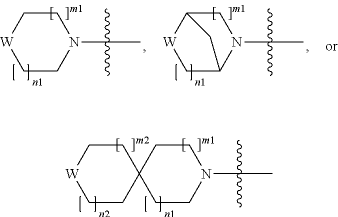

ring may each independently be substituted with —(C₁-C₄alkyl), —(C₁-C₄aminoalkyl), —(C₁-C₄hydroxyalkyl), —(C₁-C₄haloalkyl), or -halo;
W is NR₅, O, S, or S(═O)₂;
R₅ is —(C₁-C₄alkyl) or 3-6 membered heterocycloalkyl; and
n1, n2, m1, and m2 are each independently 0, 1, or 2.

According to an embodiment of the present invention, the compound represented by Chemical Formula I, a stereoisomer thereof, or a pharmaceutically acceptable salt thereof may be in the following ranges:
R₁ and R₂ are linked to form

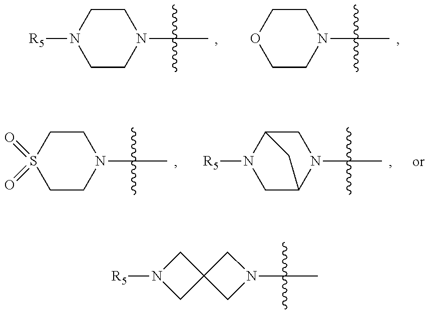

together with the N atom, wherein at least one H of the

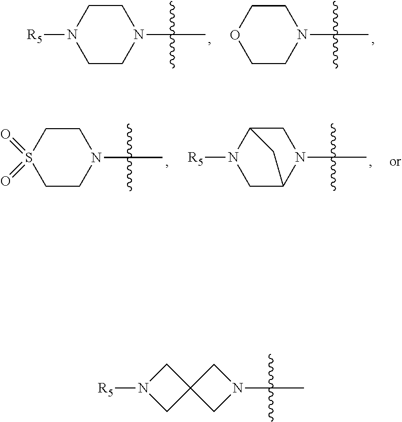

ring may each independently be substituted with —(C₁-C₄alkyl), —(C₁-C₄aminoalkyl), —(C₁-C₄hydroxyalkyl), —(C₁-C₄haloalkyl), or -halo; and
R₅ is —(C₁-C₄alkyl) or

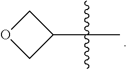 [SMILES: *C1COC1]

In addition, according to an embodiment of the present invention, specific compounds represented by Chemical Formula I of the present invention are shown in Table 1 below:

TABLE 1

| Compound | |
|---|---|
| No. | Structure |
| 1 | 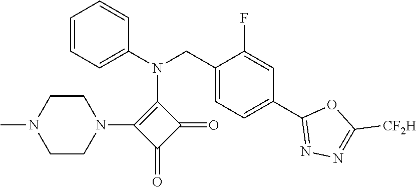 [SMILES: CN1CCN(c2c(N(Cc3ccc(-c4nnc(C(F)F)o4)cc3F)c3ccccc3)c(=O)c2=O)CC1] |
| 2 | 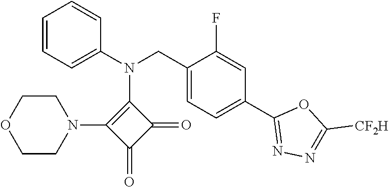 [SMILES: O=c1c(N2CCOCC2)c(N(Cc2ccc(-c3nnc(C(F)F)o3)cc2F)c2ccccc2)c1=O] |
| 3 | 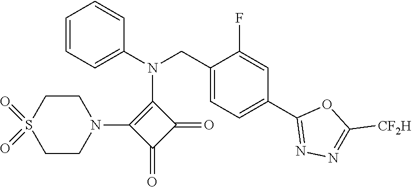 [SMILES: O=c1c(N2CCS(=O)(=O)CC2)c(N(Cc2ccc(-c3nnc(C(F)F)o3)cc2F)c2ccccc2)c1=O] |
| 4 | 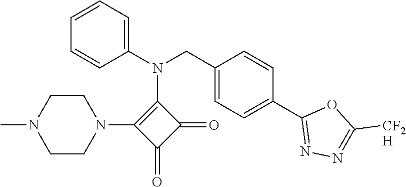 [SMILES: CN1CCN(c2c(N(Cc3ccc(-c4nnc(C(F)F)o4)cc3)c3ccccc3)c(=O)c2=O)CC1] |
| 5 | 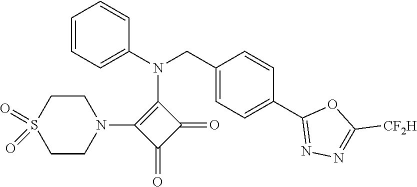 [SMILES: O=c1c(N2CCS(=O)(=O)CC2)c(N(Cc2ccc(-c3nnc(C(F)F)o3)cc2)c2ccccc2)c1=O] |
| 6 | 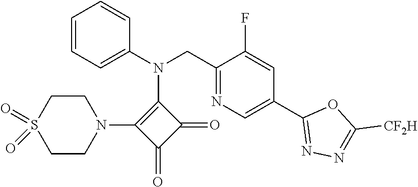 [SMILES: O=c1c(N2CCS(=O)(=O)CC2)c(N(Cc2ncc(-c3nnc(C(F)F)o3)cc2F)c2ccccc2)c1=O] |
| 7 | 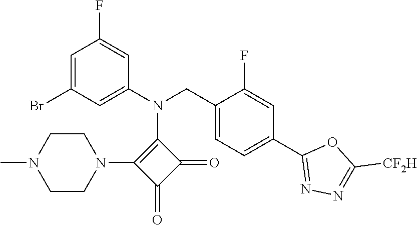 [SMILES: CN1CCN(c2c(N(Cc3ccc(-c4nnc(C(F)F)o4)cc3F)c3cc(F)cc(Br)c3)c(=O)c2=O)CC1] |
| 8 | 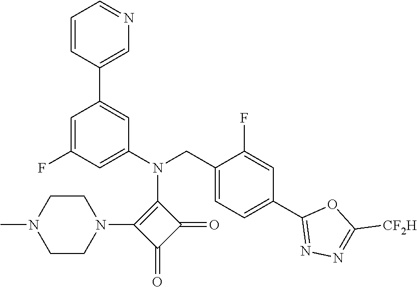 [SMILES: CN1CCN(c2c(N(Cc3ccc(-c4nnc(C(F)F)o4)cc3F)c3cc(F)cc(-c4cccnc4)c3)c(=O)c2=O)CC1] |
| 9 | 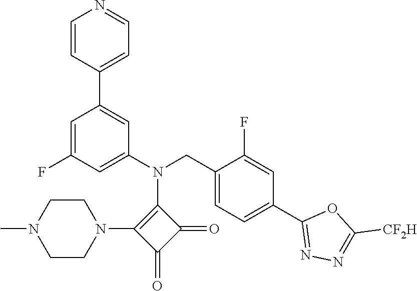 [SMILES: CN1CCN(c2c(N(Cc3ccc(-c4nnc(C(F)F)o4)cc3F)c3cc(F)cc(-c4ccncc4)c3)c(=O)c2=O)CC1] |
| 10 | 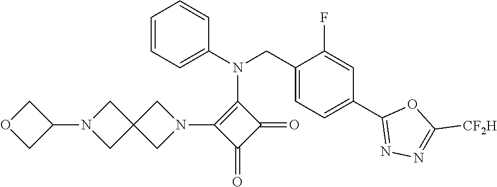 [SMILES: O=c1c(N2CC3(C2)CN(C2COC2)C3)c(N(Cc2ccc(-c3nnc(C(F)F)o3)cc2F)c2ccccc2)c1=O] |
| 11 | 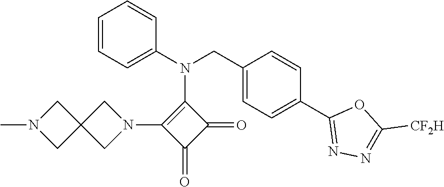 [SMILES: CN1CC2(C1)CN(c1c(N(Cc3ccc(-c4nnc(C(F)F)o4)cc3)c3ccccc3)c(=O)c1=O)C2] |
| 12 | 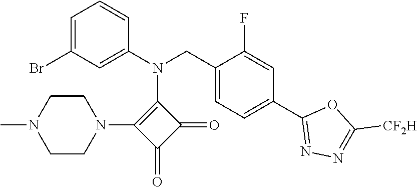 [SMILES: CN1CCN(c2c(N(Cc3ccc(-c4nnc(C(F)F)o4)cc3F)c3cccc(Br)c3)c(=O)c2=O)CC1] |
| 13 | 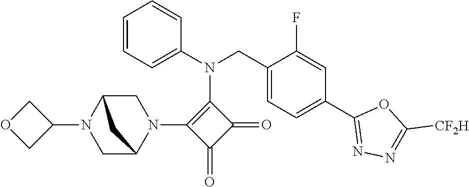 [SMILES: O=c1c(N(Cc2ccc(-c3nnc(C(F)F)o3)cc2F)c2ccccc2)c(N2C[C@@H]3C[C@H]2CN3C2COC2)c1=O] |
| 14 | 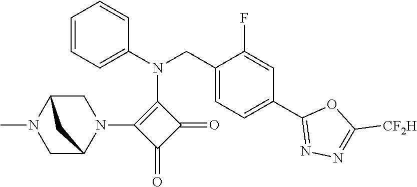 [SMILES: CN1C[C@@H]2C[C@H]1CN2c1c(N(Cc2ccc(-c3nnc(C(F)F)o3)cc2F)c2ccccc2)c(=O)c1=O] |
| 15 | 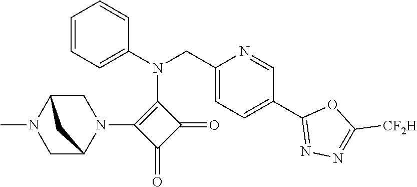 [SMILES: CN1C[C@@H]2C[C@H]1CN2c1c(N(Cc2ccc(-c3nnc(C(F)F)o3)cn2)c2ccccc2)c(=O)c1=O] |
| 16 | 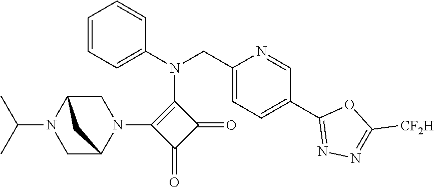 [SMILES: CC(C)N1C[C@@H]2C[C@H]1CN2c1c(N(Cc2ccc(-c3nnc(C(F)F)o3)cn2)c2ccccc2)c(=O)c1=O] |
| 17 | 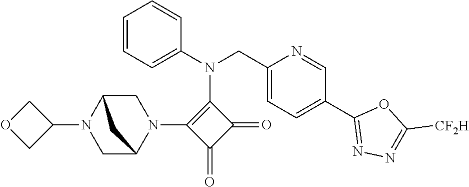 [SMILES: O=c1c(N(Cc2ccc(-c3nnc(C(F)F)o3)cn2)c2ccccc2)c(N2C[C@@H]3C[C@H]2CN3C2COC2)c1=O] |
| 18 | 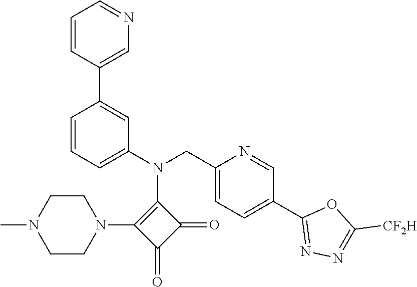 [SMILES: CN1CCN(c2c(N(Cc3ccc(-c4nnc(C(F)F)o4)cn3)c3cccc(-c4cccnc4)c3)c(=O)c2=O)CC1] |
| 19 | 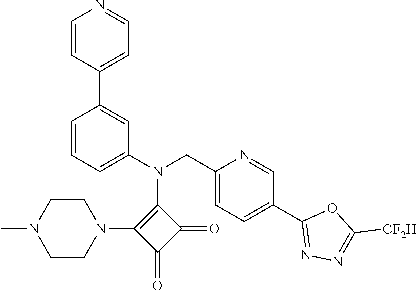 [SMILES: CN1CCN(c2c(N(Cc3ccc(-c4nnc(C(F)F)o4)cn3)c3cccc(-c4ccncc4)c3)c(=O)c2=O)CC1] |
| 20 | 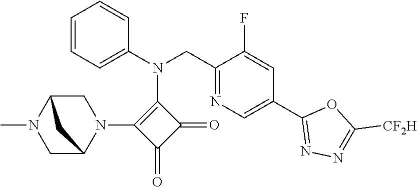 [SMILES: CN1C[C@@H]2C[C@H]1CN2c1c(N(Cc2ncc(-c3nnc(C(F)F)o3)cc2F)c2ccccc2)c(=O)c1=O] |
| 21 | 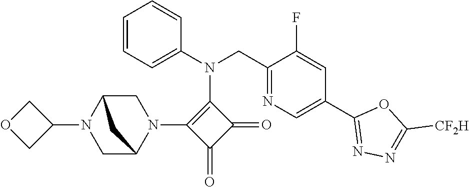 [SMILES: O=c1c(N(Cc2ncc(-c3nnc(C(F)F)o3)cc2F)c2ccccc2)c(N2C[C@@H]3C[C@H]2CN3C2COC2)c1=O] |
| 22 | 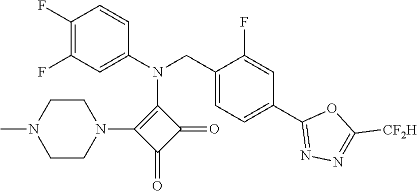 [SMILES: CN1CCN(c2c(N(Cc3ccc(-c4nnc(C(F)F)o4)cc3F)c3ccc(F)c(F)c3)c(=O)c2=O)CC1] |
| 23 | 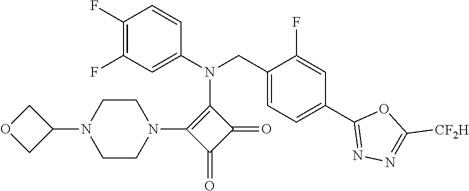 [SMILES: O=c1c(N2CCN(C3COC3)CC2)c(N(Cc2ccc(-c3nnc(C(F)F)o3)cc2F)c2ccc(F)c(F)c2)c1=O] |
| 24 | 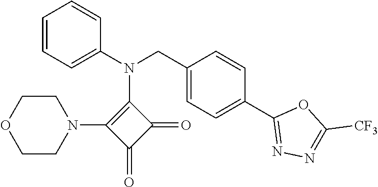 [SMILES: O=c1c(N2CCOCC2)c(N(Cc2ccc(-c3nnc(C(F)(F)F)o3)cc2)c2ccccc2)c1=O] |

In the present invention, the term “alkyl” as used herein may refer to a straight-chain or branched-chain acyclic, cyclic, or saturated hydrocarbon in which the carbon atoms are connected, unless otherwise specified. For example, “C₁₋₄ alkyl” may mean an alkyl containing 1 to 4 carbon atoms. The acyclic alkyl may include, for example, methyl, ethyl, n-propyl, n-butyl, isopropyl, sec-butyl, isobutyl, tert-butyl, and the like, but is not limited thereto. The cyclic alkyl may be used interchangeably with “cycloalkyl” in the present specification, and may include, as an example, cyclopropyl, cyclobutyl, cyclopentyl, cyclohexyl, cycloheptyl, cyclooctyl, and the like, but is not limited thereto.

In the present invention, “alkoxy” may refer to —(O-alkyl) as an alkyl ether group, wherein the alkyl is the same as defined above. For example, “C₁₋₄ alkoxy” may mean an alkoxy containing C₁₋₄ alkyl, i.e. —(O—C₁₋₄ alkyl); and examples of the alkoxy may include, but are not limited to, methoxy, ethoxy, n-propoxy, isopropoxy, n-butoxy, isobutoxy, sec-butoxy, tert-butoxy, and the like.

In the present invention, “halo” may be F, Cl, Br, or I.

In the present invention, the term “haloalkyl” may mean a straight or branched chain alkyl (hydrocarbon) having carbon atoms substituted with at least one halo as defined herein. Examples of the haloalkyl include, but are not limited to, methyl, ethyl, propyl, isopropyl, isobutyl or n-butyl independently substituted with at least one halogen such as F, Cl, Br, or I.

In the present invention, the term “hydroxyalkyl” may refer to a straight or branched chain alkyl (hydrocarbon) having carbon atoms substituted with —OH. Examples of the hydroxyalkyl include, but are not limited to, methyl, ethyl, propyl, isopropyl, isobutyl or n-butyl independently substituted with at least one hydroxy.

In the present invention, the term “aminoalkyl” may refer to a straight or branched chain alkyl (hydrocarbon) having carbon atoms substituted with amino-(NR′R″). Herein, R′ and R″ may each independently be selected from the group consisting of hydrogen and C₁₋₄ alkyl, and the selected R′ and R″ may each independently be substituted or unsubstituted.

In the present invention, the term “heterocycloalkyl” may mean a ring containing 1 to 5 heteroatoms selected from N, O and S as atoms forming the ring, and may be saturated or partially unsaturated. Herein, when unsaturated, the heterocycloalkyl may be referred to as a heterocycloalkene. Unless otherwise stated, the heterocycloalkyl may be a single ring or multiple rings such as spiro rings, bridged rings or fused rings. Further, “3 to 12-membered heterocycloalkyl” may mean a heterocycloalkyl containing 3 to 12 atoms forming a ring. Examples of the heterocycloalkyl may include, but are not limited to, pyrrolidine, piperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, quinuclidine, tropane, 2-azaspiro[3.3]heptane, (1R,5S)-3-azabicyclo[3.2.1]octane, (1s,4s)-2-azabicyclo[2.2.2]octane, or (1R,4R)-2-oxa-5-azabicyclo[2.2.2]octane, and the like.

In the present invention, “arene” may mean an aromatic hydrocarbon ring. The arene may be monocyclic arene or polycyclic arene. The number of ring carbon atoms in the arene may be 5 or more and 30 or less, 5 or more and 20 or less, or 5 or more and 15 or less. Examples of arene may include, but are not limited to, benzene, naphthalene, fluorene, anthracene, phenanthrene, bibenzene, terbenzene, quaterbenzene, quinquebenzene, sexibenzene, triphenylene, pyrene, benzofluoranthene, chrysene, and the like. In the present specification, a moiety obtained by removing one hydrogen atom from the above “arene” is referred to as “aryl”.

In the present invention, “heteroarene” may be a ring containing one or more of O, N, P, Si, and S as heterogeneous elements. The number of ring carbon atoms of the heteroarene may be 2 or more and 30 or less, or 2 or more and 20 or less. The heteroarene may be a monocyclic heteroarene or a polycyclic heteroarene. The polycyclic heteroarene may have, for example, a bicyclic or tricyclic structure. Examples of the heteroarene include, but not limited to, thiophene, purine, pyrrole, pyrazole, imidazole, thiazole, oxazole, isothiazole, oxadiazole, triazole, pyridine, bipyridyl, triazine, acridyl, pyridazine, pyrazine, quinoline, quinazoline, quinoxaline, phenoxazine, phthalazine, pyrimidine, pyridopyrimidine, pyridopyrazine, pyrazinopyrazine, isoquinoline, indole, carbazole, imidazopyridazine, imidazopyridine, imidazopyrimidine, pyrazolopyrimidine, imidazopyrazine or pyrazolopyridine, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, isoxazole, oxadiazole, thiadiazole, benzothiazole, tetrazole, phenothiazine, dibenzosilole, dibenzofuran, and the like. In an embodiment of the present invention, the heteroarene may also include a bicyclic heterocyclo-arene including an arene ring fused to a heterocycloalkyl ring or a heteroarene fused to a cycloalkyl ring. In the present specification, a moiety obtained by removing one hydrogen atom from the above “heteroarene” is referred to as “heteroaryl”.

The compound represented by Chemical Formula I of the present invention may contain at least one asymmetric carbon and thus may be present as a racemate, a racemic mixture, a single enantiomer, a diastereomeric mixture and each diastereomer. These stereoisomers may be separated by conventional techniques, and for example, the compound represented by Chemical Formula I may be separated by column chromatography, HPLC, or the like. Otherwise, each stereoisomer of the compound represented by Chemical Formula I may be stereospecifically synthesized using optically pure starting materials and/or reagents with known configurations.

In the present invention, the term “enantiomer” as used herein means a compound having the same chemical formula or molecular formula but different in spatial arrangement, or a salt thereof. Each of these enantiomers and mixtures thereof are also included within the scope of the present invention. Unless otherwise specified, a solid bond (—) connected to an asymmetric carbon atom may include a wedged solid bond ( [symbol] ) or wedge dashed bond ( [symbol] ) representing the absolute arrangement of stereocenters.

The compound represented by Chemical Formula I of the present invention may be present in the form of a “pharmaceutically acceptable salt”. As the salt, an acid addition salt formed by a pharmaceutically acceptable free acid is useful. The term “pharmaceutically acceptable salt” as used herein refers to a concentration having a relatively non-toxic and harmless effective effect on patients, which includes any organic acid or inorganic acid addition salt of the compound represented by Chemical Formula I in which side effects caused by these salts do not reduce the beneficial efficacy of the compound.

Acid addition salts are prepared by conventional methods, for example, by dissolving a compound in an excess aqueous acid solution and precipitating the salt using a water-miscible organic solvent, such as methanol, ethanol, acetone or acetonitrile. An equimolar amounts of the compound and an acid or alcohol in water may be heated, and then the mixture may be evaporated to dryness, or the precipitated salt may be suction filtered.

In this case, as the free acid, organic acids and inorganic acids may be used, wherein the inorganic acid may include hydrochloric acid, phosphoric acid, sulfuric acid, nitric acid, and the like, and the organic acid may include methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, and the like. However, the organic acid and inorganic acid are not limited thereto.

Further, a pharmaceutically acceptable metal salt may be prepared using a base. The alkali metal salt or alkaline earth metal salt is obtained, for example, by dissolving the compound in an excess alkali metal hydroxide or alkaline earth metal hydroxide solution, and filtering an insoluble compound salt, followed by evaporating and drying the filtrate. Here, as the metal salt, sodium, potassium, or calcium salt is particularly suitable in a pharmaceutical aspect, but the metal salt is not limited thereto. In addition, the corresponding silver salt may be obtained by reacting an alkali metal or alkaline earth metal salt with a suitable silver salt (for example, silver nitrate).

The pharmaceutically acceptable salt of the present invention, unless otherwise indicated, includes salts of acidic or basic groups that may be present in the compounds represented by Chemical Formula I above. For example, the pharmaceutically acceptable salt may include sodium, calcium, and potassium salts of a hydroxy group, and other pharmaceutically acceptable salts of an amino group may include hydrobromide, sulfate, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate) salts, and the like, which may be prepared through a salt preparation method known in the art.

Method for Preparing 1,3,4-oxadiazole Derivative Compound

The present invention provides a method for preparing a 1,3,4-oxadiazole derivative compound represented by the following Chemical Formula I, a stereoisomer thereof, or a pharmaceutically acceptable salt thereof:

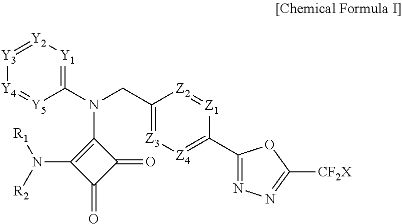

The Chemical Formula I is as defined above.

In the present invention, a preferred method for preparing the oxadiazole derivative compound represented by Chemical Formula I, a stereoisomer thereof or a pharmaceutically acceptable salt thereof are shown in [Reaction Scheme 1] to [Reaction Scheme 4], which may also include preparation methods modified to a level obvious to those skilled in the art.

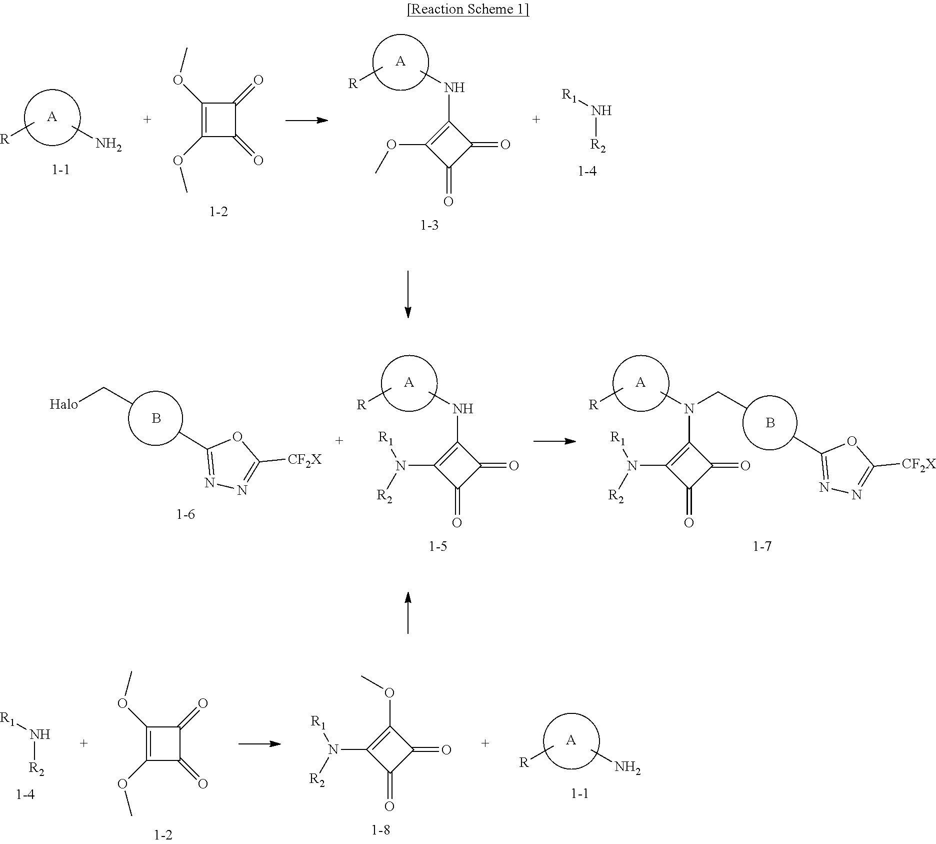

[Reaction Scheme 1]above is a method for synthesizing a 1,3,4-oxadiazole derivative compound having a cyclobutenedione structure. First, a compound represented by Chemical Formula 1-1 containing an amine group is reacted with a compound represented by Chemical Formula 1-2 to prepare a compound represented by Chemical Formula 1-3 containing an amine substituent. Then, the compound represented by Chemical Formula 1-3 is reacted with a compound represented by Chemical Formula 1-4 to prepare a compound represented by Chemical Formula 1-5 having a cyclobutenedione structure containing a diamine substituent. The compound represented by Chemical Formula 1-5 and the compound represented by Chemical Formula 1-6 are subjected to substitution reaction to thereby prepare a compound represented by Chemical Formula 1-7. Meanwhile, the compound represented by Chemical Formula 1-5 may be prepared by reacting the compound represented by Chemical Formula 1-4 with the compound represented by Chemical Formula 1-2 to prepare a compound represented by Chemical Formula 1-8, followed by a reaction with the compound represented by Chemical Formula 1-1. In the present invention, compounds 2, 3, 4, 5, 6, 7, 10, and 24 may be prepared through Reaction Scheme 1 above.

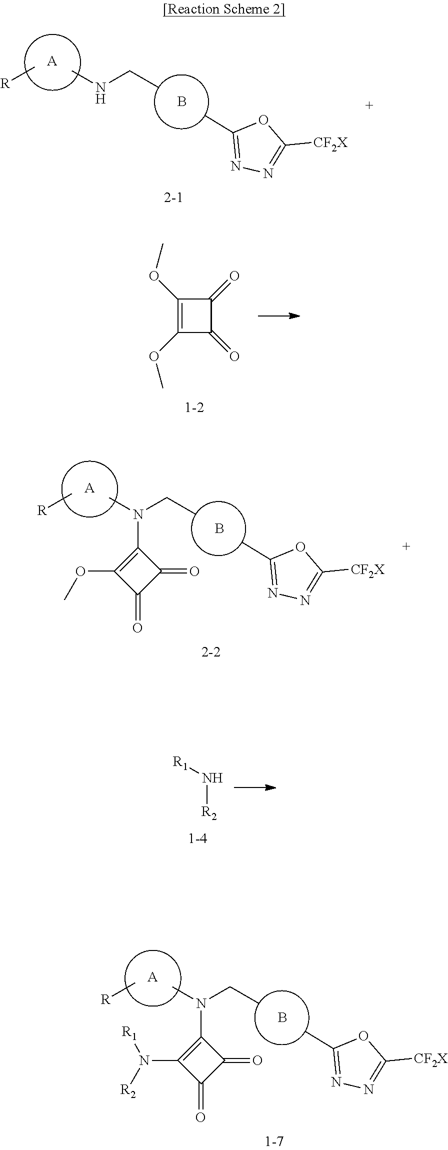

[Reaction Scheme 2]above is another synthetic method for synthesizing a 1,3,4-oxadiazole derivative compound having a cyclobutenedione structure. First, a compound represented by Chemical Formula 2-1 is reacted with a compound represented by Chemical Formula 1-2 to prepare a compound represented by Chemical Formula 2-2 containing an amine substituent. Then, the compound represented by Chemical Formula 2-2 is reacted with a compound represented by Chemical Formula 1-4 to prepare a compound represented by Chemical Formula 1-7 having a cyclobutenedione structure containing a diamine substituent. In the present invention, it is possible to prepare Compound 1, and the like, through Reaction Scheme 2 above.

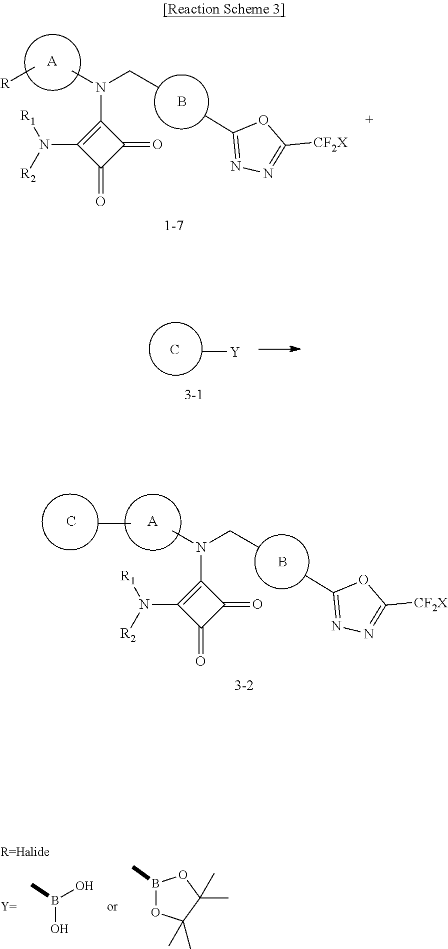

[Reaction Scheme 3]above is still another method for synthesizing a 1,3,4-oxadiazole derivative compound having a cyclobutenedione structure. The compound represented by Chemical Formula 1-7 prepared in [Reaction Scheme 1] and a compound represented by Chemical Formula 3-1 are subjected to C—C coupling (Suzuki reaction) to prepare a compound represented by Chemical Formula 3-2. In the present invention, it is possible to prepare Compounds 8 and 9, and the like, through Reaction Scheme 3 above.

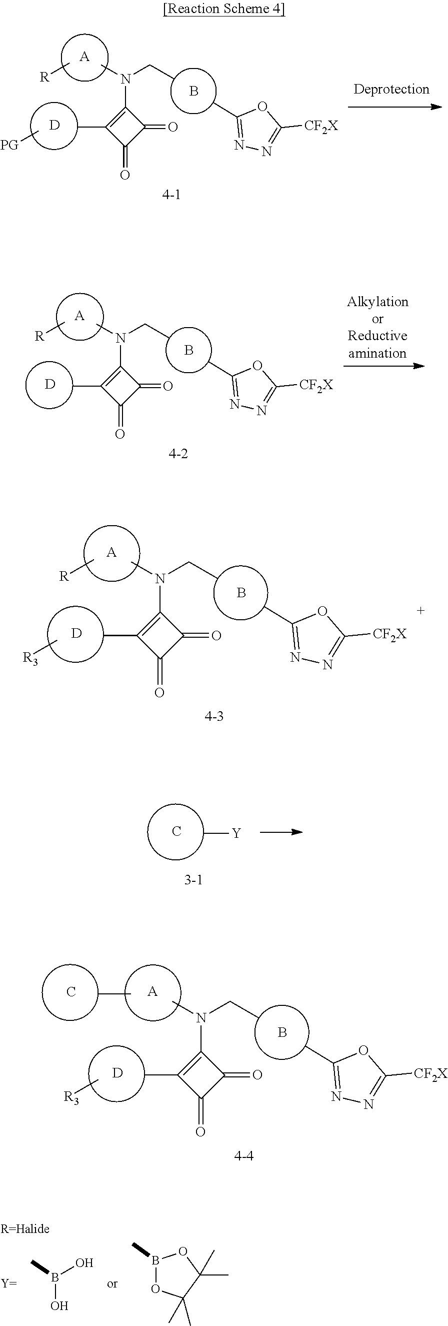

[Reaction Scheme 4]above is still another method for synthesizing a 1,3,4-oxadiazole derivative compound having a cyclobutenedione structure. A compound represented by Chemical Formula 4-2 is prepared by removing the protecting group from a compound represented by Chemical Formula 4-1 having a protecting group prepared in [Reaction Scheme 1]. Then, a compound represented by Chemical Formula 4-3 is prepared using an alkylation reaction or a reduction reaction. The compound represented by Chemical Formula 4-3 and the compound represented by Chemical Formula 3-1 are subjected to C—C coupling (Suzuki reaction) to prepare a compound represented by Chemical Formula 4-4. In the present invention, Reaction Scheme 4 above may be employed to prepare the compounds represented by Chemical Formula 4-3, i.e., Compounds 10, 11, 12, 13, 14, 15, 16, 17, 20, 21, 22, and 23, and the like, and to prepare the Compounds represented by Chemical Formula 4-4, i.e., Compounds 18 and 19, and the like.

Uses of 1.3.4-oxadiazole Derivative Compound

The present invention provides uses of a compound represented by the following Chemical Formula I, a stereoisomer thereof or a pharmaceutically acceptable salt thereof:

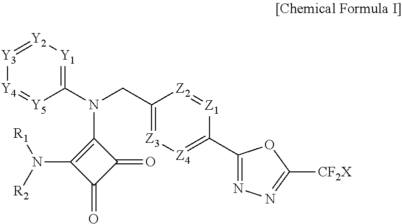

The Chemical Formula I is as defined above.

According to an embodiment of the present invention, the present invention provides a pharmaceutical composition for preventing or treating histone deacetylase 6 activity-related diseases, comprising the compound represented by Chemical Formula I as described above, a stereoisomer thereof, or a pharmaceutically acceptable salt thereof as an active ingredient. The pharmaceutical composition of the present invention selectively inhibits histone deacetylase 6, thereby exhibiting remarkable effects in preventing or treating histone deacetylase 6 activity-related diseases.

The histone deacetylase 6 activity-related diseases include infectious diseases such as prion disease; neoplasm such as benign tumors (for example, myelodysplastic syndrome) or malignant tumors (for example, multiple myeloma, lymphoma, leukemia, lung cancer, colorectal cancer, colon cancer, prostate cancer, urinary tract epithelial cell carcinoma, breast cancer, melanoma, skin cancer, liver cancer, brain cancer, stomach cancer, ovarian cancer, pancreatic cancer, head and neck cancer, oral cancer or glioma); endocrine, nutritional and metabolic diseases such as Wilson's disease, amyloidosis or diabetes; mental and behavioral disorders such as depression or Rett syndrome; neurological diseases such as central nervous system atrophy (for example, Huntington's disease, spinal muscular atrophy (SMA), spinal cerebellar ataxia (SCA)), neurodegenerative diseases (for example, Alzheimer's disease), movement disorders (for example, Parkinson's disease), neuropathy (for example, hereditary neuropathy (Charcot-Marie-Tooth disease), sporadic neuropathy, inflammatory neuropathy, drug-induced neuropathy), motor neuropathy (for example, amyotrophic lateral sclerosis (ALS)), and central nervous system demyelination (for example, multiple sclerosis (MS)); eye and ocular adnexal disease such as uveitis; circulatory diseases such as atrial fibrillation or stroke; respiratory diseases such as asthma; digestive diseases such as alcoholic liver disease, inflammatory bowel disease, Crohn's disease, ulcerative bowel disease and the like; skin and subcutaneous tissue diseases such as psoriasis; musculoskeletal and connective tissue diseases such as rheumatoid arthritis, osteoarthritis or systemic lupus erythematosus (SLE); or congenital malformations, alterations and chromosomal abnormalities such as autosomal dominant polycystic kidney disease, and in addition thereto include symptoms or diseases related to the abnormal function of histone deacetylases.

The stereoisomers and pharmaceutically acceptable salts are as described above in the stereoisomers and pharmaceutically acceptable salts of the compound represented by Chemical Formula I of the present invention.

For administration, the pharmaceutical composition of the present invention may further comprise at least one or more pharmaceutically acceptable carrier in addition to the compound represented by Chemical Formula I, a stereoisomer or a pharmaceutically acceptable salt thereof. The pharmaceutically acceptable carrier may be saline, sterile water, Ringer's solution, buffered saline, dextrose solution, maltodextrin solution, glycerol, ethanol, or a mixture of one or more of these ingredients, and, if necessary, may contain other conventional additives such as antioxidants, buffers, bacteriostatic agents, and the like. Further, diluents, dispersants, surfactants, binders and lubricants may be additionally added and may be formulated into injectable formulations such as aqueous solutions, suspensions, emulsions, and the like, pills, capsules, granules or tablets. Accordingly, the pharmaceutical composition of the present invention may be a patch, liquid, pill, capsule, granule, tablet, suppository, or the like. These formulations may be prepared by conventional methods used for formulation in the art or a method disclosed in the document [see, Remington's Pharmaceutical Science (latest edition), Mack Publishing Company, Easton PA], and may be formulated into various formulations depending on each disease or component.

The composition of the present invention may be administered orally or parenterally (for example, intravenous, subcutaneous, intraperitoneal or topical application) according to the desired method, and the dosage varies depending on the patient's weight, age, sex, health condition, diet, administration time, administration method, excretion rate, and severity of the disease. The daily dosage of the compound represented by Chemical Formula I of the present invention is about 1 to 1000 mg/kg, preferably 5 to 100 mg/kg, and may be divided and administered once or several times a day.

The pharmaceutical composition of the present invention may further comprise at least one active ingredient exhibiting the same or similar efficacy in addition to the compound represented by Chemical Formula I, a stereoisomer or a pharmaceutically acceptable salt thereof.

According to an embodiment of the present invention, the present invention provides a method for preventing or treating histone deacetylase 6 activity-related diseases, comprising: administering a therapeutically effective amount of the compound represented by Chemical Formula I, a stereoisomer thereof, or a pharmaceutically acceptable salt thereof to a subject in need thereof. The subject may be a mammal including a human.

The method for preventing or treating histone deacetylase 6 activity-related diseases comprises not only dealing with the disease itself prior to the onset of symptoms, but also inhibiting or avoiding symptoms thereof by administering the compound represented by Chemical Formula I, a stereoisomer or a pharmaceutically acceptable salt thereof. In addition, the method for preventing or treating histone deacetylase 6 activity-related diseases of the present invention may further administering a therapeutically effective amount of an additional active agent that is helpful in treating the diseases together with the compound represented by Chemical Formula I, wherein the additional active agent may exhibit synergistic or adjuvant effects together with the compound represented by Chemical Formula I above.

The term “therapeutically effective amount” used herein refers to an amount of the compound represented by Chemical Formula I effective for the treatment or prevention of histone deacetylase 6 activity-related diseases. Specifically, the “pharmaceutically effective amount” means an amount sufficient to treat a disease at a reasonable benefit/risk ratio applicable to medical treatment, and the effective dose level may be determined depending on factors including the subject type and severity, age, sex, type of diseases, the activity of the drug, the sensitivity to the drug, the time of administration, the route of administration, the rate of excretion, the duration of treatment, drugs used concurrently, and other factors well known in the medical field. The pharmaceutical composition of the present invention may be administered as an individual therapeutic agent or in combination with other therapeutic agents, and may be administered sequentially or simultaneously with a commercially available therapeutic agent. In addition, the pharmaceutical composition of the present invention may be administered singly or in multiple doses. In consideration of all of the above factors, it is important to administer an amount capable of obtaining the maximum effect with the minimum amount without side effects, which may be easily determined by those skilled in the art. The administration dose of the pharmaceutical composition of the present invention may be determined by specialists according to various factors such as the patient's condition, age, sex, complications, and the like. Since the active ingredient of the pharmaceutical composition of the present invention has excellent safety, the active ingredient may be used even above the predetermined dose.

According to an embodiment of the present invention, the present invention provides a method for selectively inhibiting histone deacetylase 6 (HDAC6) by administering the compound represented by Chemical Formula I, a stereoisomer thereof, or a pharmaceutically acceptable salt thereof to mammals, including humans.

According to an embodiment of the present invention, the present invention provides uses of a compound represented by Chemical Formula I, a stereoisomer thereof, or a pharmaceutically acceptable salt thereof for the manufacture of a medicament for the treatment of histone deacetylase 6 activity-related diseases. The compound represented by Chemical Formula I for the manufacture of medicaments may be mixed with acceptable adjuvants, diluents, carriers, and the like, and may be prepared as a combined preparation with other active agents to have a synergistic effect of the active ingredients.

Matters mentioned in the uses, compositions and treatment methods of the present invention are applied equally as long as they do not contradict each other.

Exemplary embodiments of the present invention may be modified in various other forms, and the scope of the present invention is not limited to the exemplary embodiments described below. In addition, the exemplary embodiments of the present invention are provided to more completely explain the present invention to an ordinary person skilled in the art. Further, “including” a component throughout the specification does not mean excluding other components, but rather it means that other components may be further included, unless otherwise stated.

### Advantageous Effects of Invention

The compound represented by Chemical Formula I, a stereoisomer thereof, or a pharmaceutically acceptable salt thereof according to the present invention may selectively inhibit HDAC6 to thereby have a remarkably excellent preventive or therapeutic effect on histone deacetylase 6 activity-related diseases.

### BEST MODE FOR CARRYING OUT THE INVENTION

Hereinafter, the present invention will be described in more detail through Examples and Experimental Examples. However, these Examples and the like are only presented as examples of the present invention, and the scope of the present invention is not limited only to these Examples.

Preparation of 1,3,4-oxadiazole Derivative Compound

A specific method for preparing a compound represented by Chemical Formula I is described as follows.

### Example 1: Synthesis of 3-((4-(5-(difluoromethyl)-1,3,4-oxadiazol-2-yl)-2-fluorobenzyl)(phenyl)amino)-4-(4-methylpiperazin-1-yl)cyclobut-3-ene-1,2-dione (Compound 1)

### [Step 1] Synthesis of 3-((4-(5-(difluoromethyl)-1,3,4-oxadiazol-2-yl)-2-fluorobenzyl)(phenyl)amino)-4-methoxycyclobut-3-ene-1.2-dione

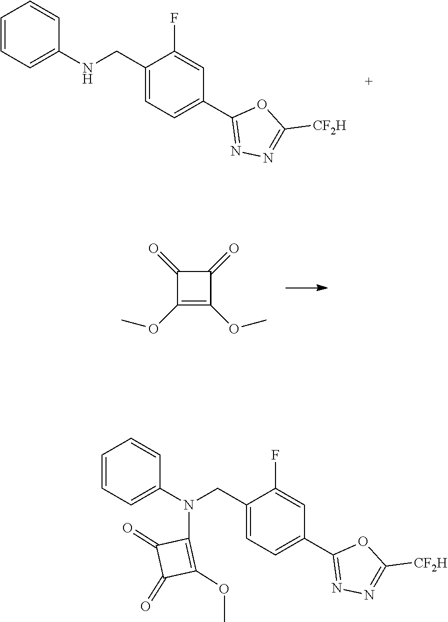

N-(4-(5-(difluoromethyl)-1,3,4-oxadiazol-2-yl)-2-fluorobenzyl)aniline (0.200 g, 0.626 mmol) and 3,4-dimethoxycyclobut-3-ene-1,2-dione (0.098 g, 0.689 mmol) were dissolved in methanol (5 mL) at room temperature, and the reaction solution was stirred at 60° C. for 18 hours. Then, the reaction was completed by lowering the reaction temperature to room temperature. The reaction mixture was filtered through a glass filter to obtain a filtrate from which solids were removed, and the solvent was removed from the filtrate under reduced pressure. Then, the concentrate was purified by column chromatography (SiO₂, 4 g cartridge; ethyl acetate/hexane=10% to 50%) and concentrated to obtain the title compound (0.069 g, 25.7%) as a yellow oil.

### [Step 2] Synthesis of Compound 1

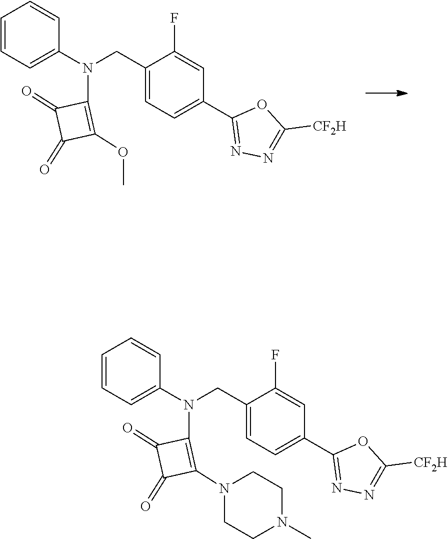 [SMILES: COc1c(N(Cc2ccc(-c3nnc(C(F)F)o3)cc2F)c2ccccc2)c(=O)c1=O ; CN1CCN(c2c(N(Cc3ccc(-c4nnc(C(F)F)o4)cc3F)c3ccccc3)c(=O)c2=O)CC1]

3-((4-(5-(Difluoromethyl)-1,3,4-oxadiazol-2-yl)-2-fluorobenzyl)(phenyl)amino)-4-m ethoxycyclobut-3-ene-1,2-dione (0.100 g, 0.233 mmol) prepared in Step 1 and 1-methylpiperazine (0.052 mL, 0.466 mmol) were dissolved in methanol (3 mL) at room temperature, and the reaction solution was stirred at 60° C. for 18 hours. Then, the reaction was completed by lowering the reaction temperature to room temperature. After removing the solvent from the reaction mixture under reduced pressure, the concentrate was purified by column chromatography (SiO₂, 4 g cartridge; methanol/dichloromethane=0% to 5%) and concentrated to obtain the title compound (0.016 g, 13.8%) as a pale yellow solid.

¹H NMR (400 MHz, CDCl₃) δ 7.88 (d, J=8.0 Hz, 1H), 7.77 (d, J=8.7 Hz, 1H), 7.63 (t, J=7.5 Hz, 1H), 7.36 (t, J=7.9 Hz, 2H), 7.19 (t, J=7.4 Hz, 1H), 7.04-6.79 (m, 3H), 5.58 (s, 2H), 3.28 (brs, 4H), 2.25 (brs, 4H), 2.22 (s, 3H); LRMS (ES) m/z 498.5 (M⁺+1).

### Example 2: Synthesis of 3-((4-(5-(difluoromethyl)-1,3,4-oxadiazol-2-yl)-2-fluorobenzyl)(phenyl)amino)-4-morpholinocyclobut-3-ene-1,2-dione (Compound 2)

### [Step 1] Synthesis of 3-morpholino-4-(phenylamino)cyclobut-3-ene-1,2-dione

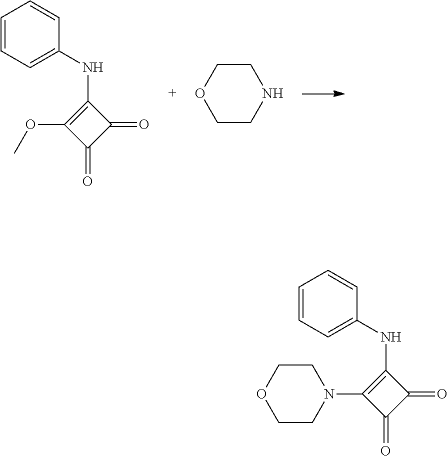 [SMILES: COc1c(Nc2ccccc2)c(=O)c1=O ; C1COCCN1 ; O=c1c(Nc2ccccc2)c(N2CCOCC2)c1=O]

3-Methoxy-4-(phenylamino)cyclobut-3-ene-1,2-dione (0.500 g, 2.461 mmol), morpholine (0.213 mL, 2.461 mmol), and N,N-diisopropylethylamine (0.429 mL, 2.461 mmol) were dissolved in methanol (10 mL) at 50° C., and the reaction solution was stirred for 2 hours at room temperature. Water was poured into the reaction mixture, and the mixture was extracted with dichloromethane. The organic layer was washed with a saturated aqueous sodium chloride solution, dried over anhydrous sodium sulfate, filtered, and concentrated under reduced pressure. Diethyl ether was added to the concentrate and stirred to precipitate a solid, and the precipitated solid was filtered, washed with diethyl ether, and dried to obtain the title compound (0.445 g, 70.0%) as a white solid.

### [Step 2] Synthesis of Compound 2

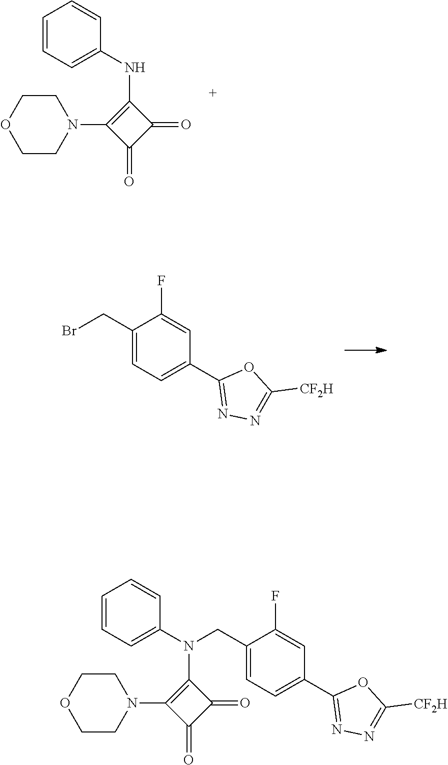 [SMILES: O=c1c(Nc2ccccc2)c(N2CCOCC2)c1=O ; Fc1cc(-c2nnc(C(F)F)o2)ccc1CBr ; O=c1c(N2CCOCC2)c(N(Cc2ccc(-c3nnc(C(F)F)o3)cc2F)c2ccccc2)c1=O]

3-Morpholino-4-(phenylamino)cyclobut-3-ene-1,2-dione (0.100 g, 0.387 mmol) prepared in Step 1 and sodium hydride (60.00%, 0.017 g, 0.426 mmol) were dissolved in N,N-dimethylformamide (3 mL) at 0° C. To the reaction solution, 2-(4-(bromomethyl)-3-fluorophenyl)-5-(difluoromethyl)-1,3,4-oxadiazole (0.131 g, 0.426 mmol) was added and stirred for 1 hour at room temperature. The solvent was removed from the reaction mixture under reduced pressure to obtain a concentrate. Into the concentrate, a saturated aqueous solution of sodium hydrogen carbonate was poured, and the reaction mixture was extracted with dichloromethane. The mixture was filtered through a plastic filter to remove a solid residue and an aqueous layer and then concentrated under reduced pressure. The concentrate was purified by column chromatography (SiO₂, 4 g cartridge; ethyl acetate/hexane=20% to 70%) and concentrated to obtain the title compound (0.019 g, 10.1%) as a white solid.

¹H NMR (400 MHz, CDCl₃) δ 7.88 (d, J=8.1 Hz, 1H), 7.77 (d, J=10.1 Hz, 1H), 7.64 (t, J=7.6 Hz, 1H), 7.38 (t, J=7.8 Hz, 2H), 7.22 (t, J=7.4 Hz, 1H), 7.05-6.79 (m, 3H), 5.59 (s, 2H), 3.52-3.50 (m, 4H), 3.26 (brs, 4H); LRMS (ES) m/z 485.4 (M⁺+1).

### Example 3: Synthesis of 3-((4-(5-(difluoromethyl)-1,3,4-oxadiazol-2-yl)-2-fluorobenzyl)(phenyl)amino)-4-(1,1-dioxidothiomorpholino)cyclobut-3-ene-1,2-dione (Compound 3)

### [Step 1] Synthesis of 3-(1,1-dioxidothiomorpholino)-4-(phenylamino)cyclobut-3-ene-1,2-dione

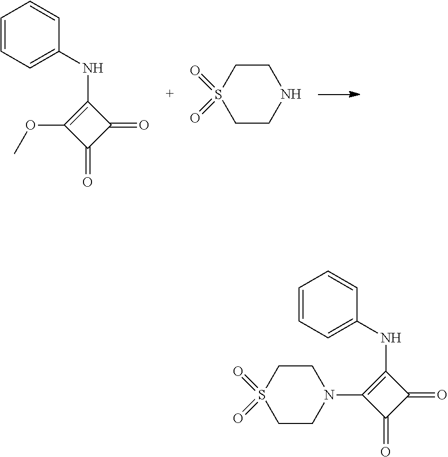 [SMILES: COc1c(Nc2ccccc2)c(=O)c1=O ; O=S1(=O)CCNCC1 ; O=c1c(Nc2ccccc2)c(N2CCS(=O)(=O)CC2)c1=O]

3-Methoxy-4-(phenylamino)cyclobut-3-ene-1,2-dione (0.500 g, 2.461 mmol), thiomorpholine 1,1-dioxide (0.333 g, 2.461 mmol), and N,N-diisopropylethylamine (0.429 mL, 2.461 mmol) were dissolved in methanol (10 mL) at 50° C., and the reaction solution was stirred at room temperature for 4 hours. Water was poured into the reaction mixture, and the mixture was extracted with dichloromethane. The organic layer was washed with a saturated aqueous sodium chloride solution, dried over anhydrous sodium sulfate, filtered, and concentrated under reduced pressure. The precipitated solid was filtered, washed with methanol and dried to obtain the title compound (0.554 g, 73.5%) as a pale yellow solid.

### [Step 2] Synthesis of Compound 3

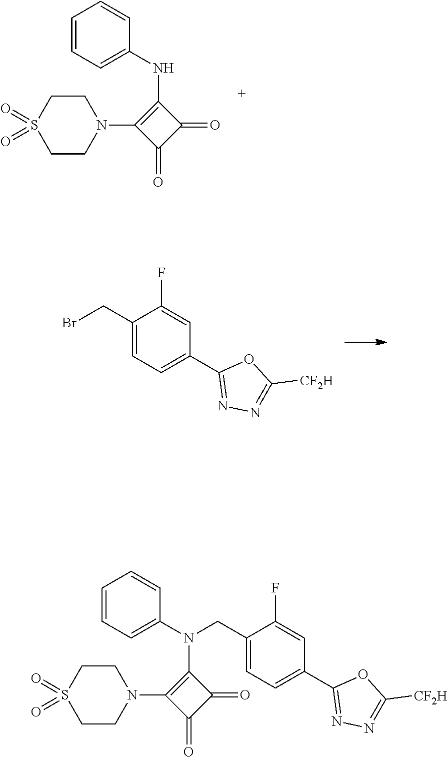 [SMILES: O=c1c(Nc2ccccc2)c(N2CCS(=O)(=O)CC2)c1=O ; Fc1cc(-c2nnc(C(F)F)o2)ccc1CBr ; O=c1c(N2CCS(=O)(=O)CC2)c(N(Cc2ccc(-c3nnc(C(F)F)o3)cc2F)c2ccccc2)c1=O]

3-(1,1-Dioxidothiomorpholino)-4-(phenylamino)cyclobut-3-ene-1,2-dione (0.100 g, 0.326 mmol) prepared in Step 1 and sodium hydride (60.00%, 0.014 g, 0.359 mmol) were dissolved in N,N-dimethylformamide (3 mL) at 0° C. To the reaction solution, 2-(4-(bromomethyl)-3-fluorophenyl)-5-(difluoromethyl)-1,3,4-oxadiazole (0.110 g, 0.359 mmol) was added and stirred at room temperature for 1 hour. The solvent was removed from the reaction mixture under reduced pressure to obtain a concentrate. Into the concentrate, a saturated aqueous sodium chloride solution was poured, and the reaction mixture was extracted with dichloromethane. The organic layer was washed with a saturated aqueous sodium chloride solution, dried over anhydrous calcium chloride (II), filtered, and concentrated under reduced pressure. The concentrate was purified by column chromatography (SiO₂, 4 g cartridge; ethyl acetate/hexane=10% to 40%) and concentrated to obtain the title compound (0.023 g, 13.2%) as a pale yellow solid.

¹H NMR (400 MHz, CDCl₃) δ 7.89 (dd, J=8.0, 1.4 Hz, 1H), 7.77 (dd, J=10.0, 1.4 Hz, 1H), 7.61 (t, J=7.6 Hz, 1H), 7.43 (t, J=7.8 Hz, 2H), 7.31 (d, J=7.4 Hz, 1H), 7.09 (d, J=7.8 Hz, 2H), 7.05-6.79 (m, 1H), 5.60 (s, 2H), 3.68 (brs, 4H), 2.89-2.87 (m, 4H); LRMS (ES) m/z 533.5 (M⁺+1).

### Example 4: Synthesis of 3-((4-(5-(difluoromethyl)-1,3,4-oxadiazol-2-yl)benzyl)(phenyl)amino)-4-(4-methylpiperazin-1-yl)cyclobut-3-ene-1,2-dione (Compound 4)

### [Step 1] Synthesis of tert-butyl 4-(2-methoxy-3,4-dioxocyclobut-1-en-1-yl)piperazine-1-carboxylate

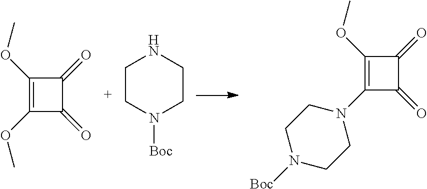

3,4-Dimethoxycyclobut-3-ene-1,2-dione (1.520 g, 10.696 mmol) and tertbutylpiperazine-1-carboxylate (1.992 g, 10.696 mmol) were dissolved in ethanol (30 mL) at room temperature, and the reaction solution was stirred at the same temperature for 18 hours. The precipitated solid was filtered, washed with hexane, and dried to obtain the desired title compound (1.860 g, 58.7%) as a white solid.

### [Step 2] Synthesis of tert-butyl 4-(3,4-dioxo-2-(phenylamino)cyclobut-1-ene-1-yl)piperazine-1-carboxylate

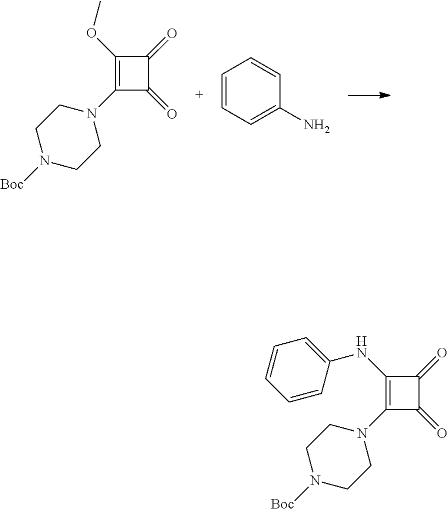 [SMILES: COc1c(N2CCN(C(=O)OC(C)(C)C)CC2)c(=O)c1=O ; Nc1ccccc1 ; CC(C)(C)OC(=O)N1CCN(c2c(Nc3ccccc3)c(=O)c2=O)CC1]

Tert-butyl 4-(2-methoxy-3,4-dioxocyclobut-1-en-1-yl)piperazine-1-carboxylate (1.860 g, 6.277 mmol) prepared in Step 1, aniline (0.573 mL, 6.277 mmol), and triethylamine (1.750 mL, 12.554 mmol) were dissolved in ethanol (20 mL) at 78° C., and the reaction solution was stirred for 18 hours at the same temperature. Then, the reaction was completed by lowering the reaction temperature to room temperature. The solvent was removed from the reaction mixture under reduced pressure to obtain a concentrate. Into the concentrate, water was poured, followed by extraction with dichloromethane. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; ethyl acetate/hexane=0% to 50%) and concentrated to obtain the title compound (0.300 g, 13.4%) as a yellow solid.

### [Step 3] Synthesis of tert-butyl 4-(2-((4-(5-(difluoromethyl)-1,3,4-oxadiazol-2-yl)benzyl)(phenyl)amino)-3,4-dioxocyclobut-1-en-1-yl)piperazine-1-carboxylate

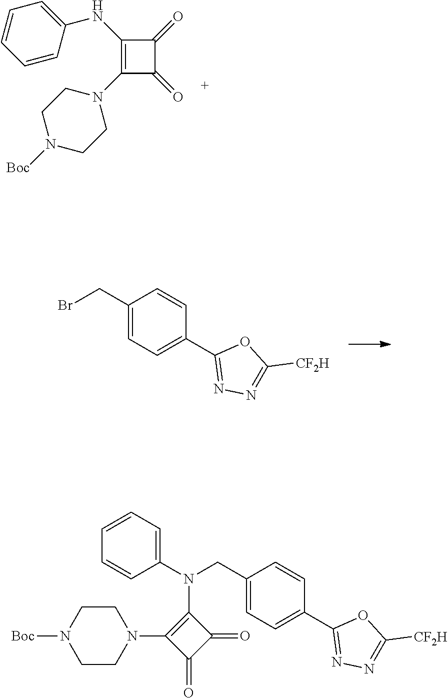 [SMILES: CC(C)(C)OC(=O)N1CCN(c2c(Nc3ccccc3)c(=O)c2=O)CC1 ; FC(F)c1nnc(-c2ccc(CBr)cc2)o1 ; CC(C)(C)OC(=O)N1CCN(c2c(N(Cc3ccc(-c4nnc(C(F)F)o4)cc3)c3ccccc3)c(=O)c2=O)CC1]

Tert-butyl 4-(3,4-dioxo-2-(phenylamino)cyclobut-1-en-1-yl)piperazine-1-carboxylate (0.176 g, 0.492 mmol) prepared in Step 2 was dissolved in N,N-dimethylformamide (10 mL) at 0° C. To the reaction solution, sodium hydride (60.00%, 0.030 g, 0.739 mmol) was added and stirred for 30 minutes at the same temperature. To the reaction mixture, 2-(4-(bromomethyl)phenyl)-5-(difluoromethyl)-1,3,4-oxadiazole (0.17 g, 0.59 mmol) was added and further stirred at room temperature for 3 hours. The solvent was removed from the reaction mixture under reduced pressure to obtain a concentrate. Into the concentrate, water was poured, followed by extraction with ethyl acetate. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; ethyl acetate/hexane=0% to 50%) and concentrated to obtain the title compound (0.135 g, 48.5%) as a white solid.

### [Step 4] Synthesis of 3-((4-(5-(difluoromethyl)-1.3.4-oxadiazol-2-yl)benzyl)(phenyl)amino)-4-(piperazin-1-yl)cyclobut-3-ene-1,2-dione 2,2,2-trifluoroacetate

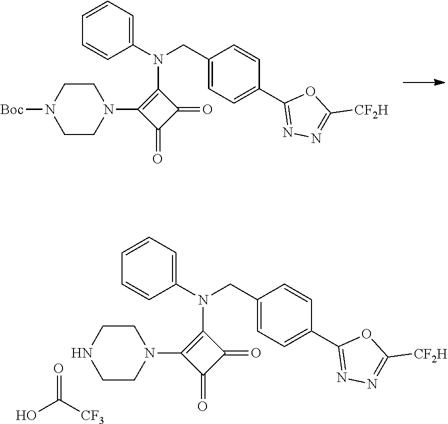

Tert-butyl 4-(2-((4-(5-(difluoromethyl)-1,3,4-oxadiazol-2-yl)benzyl)(phenyl)amino)-3,4-dioxocyclobut-1-en-1-yl)piperazine-1-carboxylate (0.135 g, 0.239 mmol) prepared in Step 3 and trifluoroacetic acid (0.183 mL, 2.387 mmol) were dissolved in dichloromethane (10 mL) at room temperature, and the reaction solution was stirred for 18 hours at the same temperature. After removing the solvent from the reaction mixture under reduced pressure, the obtained product was used without further purification (0.150 g, 108.4%, yellow oil).

### [Step 5] Synthesis of Compound 4

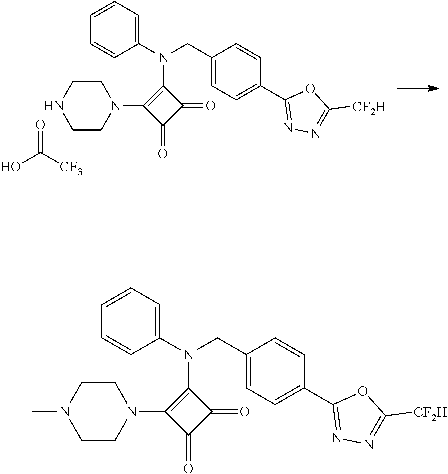

3-((4-(5-(Difluoromethyl)-1,3,4-oxadiazol-2-yl)benzyl)(phenyl)amino)-4-(piperazine-1-yl)cyclobut-3-ene-1,2-dione 2,2,2-trifluoroacetate (0.150 g, 0.259 mmol) prepared in Step 4, N,N-diisopropylethylamine (0.045 mL, 0.259 mmol), formaldehyde (0.016 g, 0.518 mmol), and sodium triacetoxyborohydride (0.110 g, 0.518 mmol) were dissolved in dichloromethane (10 mL) at room temperature. The reaction solution was stirred for 18 hours at the same temperature. Water was poured into the reaction mixture, followed by extraction with dichloromethane. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; methanol/dichloromethane=0% to 10%) and concentrated to obtain the title compound (0.066 g, 53.2%) as a white solid.

¹H NMR (400 MHz, CDCl₃) δ 8.05 (d, J=7.5 Hz, 2H), 7.47 (d, J=8.2 Hz, 2H), 7.35 (t, J=7.9 Hz, 2H), 7.20 (t, J=7.4 Hz, 1H), 7.04 (s, 0.25H), 6.98 (d, J=7.7 Hz, 2H), 6.92 (s, 0.5H), 6.79 (s, 0.25H), 5.50 (s, 2H), 3.28 (br s, 4H), 2.26 (s, 4H), 2.22 (s, 3H); LRMS (ES) m/z 480.5 (M⁺+1).

### Example 5: Synthesis of 3-((4-(5-(difluoromethyl)-1,3,4-oxadiazol-2-yl)benzyl)(phenyl)amino)-4-(1,1-dioxidothiomorpholino)cyclobut-3-ene-1,2-dione (Compound 5)

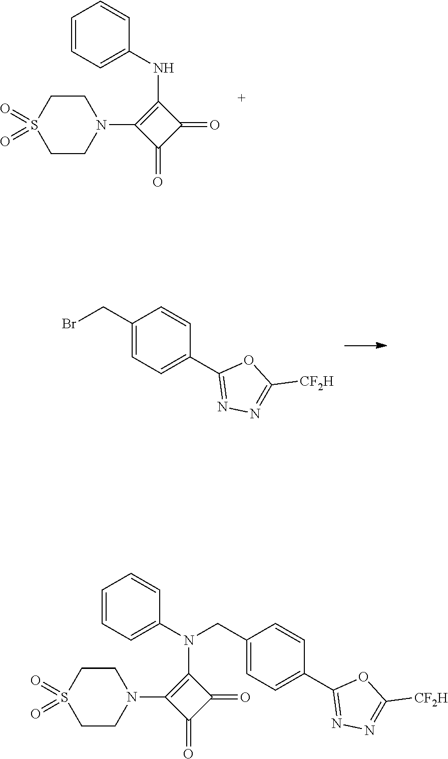 [SMILES: O=c1c(Nc2ccccc2)c(N2CCS(=O)(=O)CC2)c1=O ; FC(F)c1nnc(-c2ccc(CBr)cc2)o1 ; O=c1c(N2CCS(=O)(=O)CC2)c(N(Cc2ccc(-c3nnc(C(F)F)o3)cc2)c2ccccc2)c1=O]

3-(1,1-Dioxidothiomorpholino)-4-(phenylamino)cyclobut-3-ene-1,2-dione (0.100 g, 0.326 mmol) prepared in Step 1 of Example 3 and sodium hydride (60.00%, 0.014 g, 0.359 mmol) were dissolved in N,N-dimethylformamide (3 mL) at room temperature. To the reaction solution, 2-(4-(bromomethyl)phenyl)-5-(difluoromethyl)-1,3,4-oxadiazole (0.104 g, 0.359 mmol) was added and stirred for 3 hours at the same temperature. Water was poured into the reaction mixture, followed by extraction with dichloromethane. Then, the extract was filtered through a plastic filter to remove a solid residue and an aqueous layer, and concentrated under reduced pressure. The concentrate was purified by column chromatography (SiO₂, 4 g cartridge; ethyl acetate/hexane=10% to 40%) and concentrated. Ethyl acetate and diethyl ether were added to the obtained product and stirred. The precipitated solid was filtered, washed with diethyl ether, and dried to obtain the title compound (0.016 g, 9.5%) as a white solid.

¹H NMR (400 MHz, DMSO-d₆) δ 8.00 (d, J=8.2 Hz, 2H), 7.60-7.41 (m, 3H), 7.39 (t, J=6.4 Hz, 1H), 7.23-7.19 (m, 3H), 5.52 (s, 2H), 3.53 (brs, 4H), 3.14 (brs, 4H); LRMS (ES) m/z 515.4 (M⁺+1).

### Example 6: Synthesis of 3-(((5-(5-(difluoromethyl)-1,3,4-oxadiazol-2-yl)-3-fluoropyridin-2-yl)methyl)(phenyl)amino)-4-(1,1-dioxidothiomorpholino)cyclobut-3-ene-1,2-dione (Compound 6)

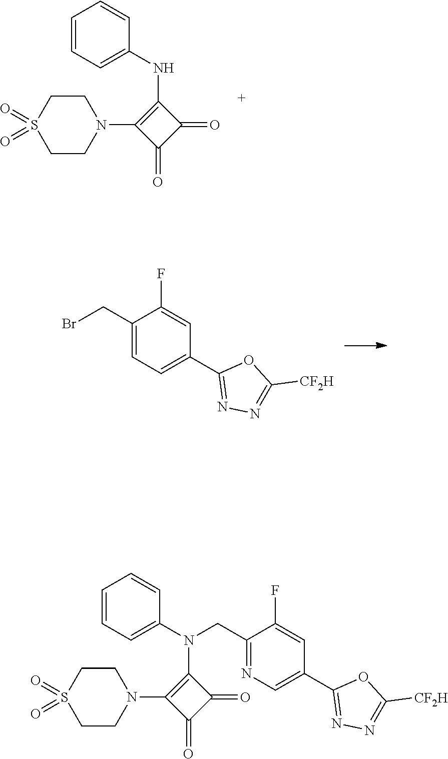 [SMILES: O=c1c(Nc2ccccc2)c(N2CCS(=O)(=O)CC2)c1=O ; Fc1cc(-c2nnc(C(F)F)o2)ccc1CBr ; O=c1c(N2CCS(=O)(=O)CC2)c(N(Cc2ncc(-c3nnc(C(F)F)o3)cc2F)c2ccccc2)c1=O]

3-(1,1-Dioxidothiomorpholino)-4-(phenylamino)cyclobut-3-ene-1,2-dione (0.100 g, 0.326 mmol) prepared in Step 1 of Example 3 and sodium hydride (60.00%, 0.014 g, 0.359 mmol) were dissolved in N,N-dimethylformamide (3 mL) at room temperature. To the reaction solution, 2-(6-(bromomethyl)-5-(fluoropyridin-3-yl)-5-(difluoromethyl)-1,3,4-oxadiazole (0.111 g, 0.359 mmol) was added and stirred for 3 hours at the same temperature. Water was poured into the reaction mixture, and the mixture was extracted with dichloromethane. The mixture was filtered through a plastic filter to remove the solid residue and the aqueous layer and then concentrated under reduced pressure. The concentrate was purified by column chromatography (SiO₂, 4 g cartridge; ethyl acetate/hexane=10% to 40%) and concentrated. Ethyl acetate and diethyl ether were added to the obtained product and stirred to precipitate a solid, and the precipitated solid was filtered, washed with diethyl ether, and dried to obtain the title compound (0.017 g, 9.8%) as a pale yellow solid.

¹H NMR (400 MHz, DMSO-d₆) δ 8.99 (s, 1H), 8.41 (dd, J=9.9, 1.5 Hz, 1H), 7.71-7.45 (m, 1H), 7.41 (t, J=7.8 Hz, 2H), 7.21-7.17 (m, 3H), 5.70 (brs, 2H), 3.62 (brs, 4H), 3.17 (s, 4H); LRMS (ES) m/z 534.4 (M⁺+1).

### Example 7: Synthesis of 3-((3-bromo-5-fluorophenyl)(4-(5-(difluoromethyl)-1,3,4-oxadiazol-2-yl)-2-fluorobenzyl)amino)-4-(4-methylpiperazin-1-yl)cyclobut-3-ene-1,2-dione (Compound 7)

### [Step 1] Synthesis of 3-((3-bromo-5-fluorophenyl)amino)-4-methoxycyclobut-3-ene-1,2-dione

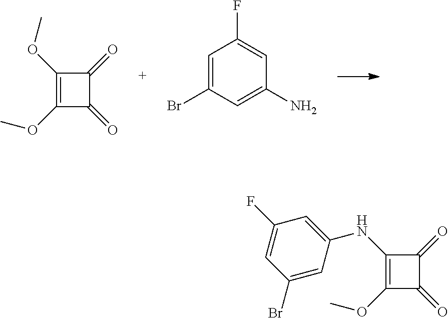

3,4-Dimethoxycyclobut-3-ene-1,2-dione (2.000 g, 14.074 mmol) and 3-bromo-5-fluoroaniline (2.674 g, 14.074 mmol) were dissolved in methanol (50 mL) at room temperature and stirred for 18 hours at the same temperature. The precipitated solid was filtered, washed with hexane and dried to obtain the title compound (3.500 g, 82.9%) as a white solid.

### [Step 2] Synthesis of tert-butyl 4-(2-((3-bromo-5-fluorophenyl)amino)-3,4-dioxocyclobut-1-en-1-yl)piperazine-1-carboxylate

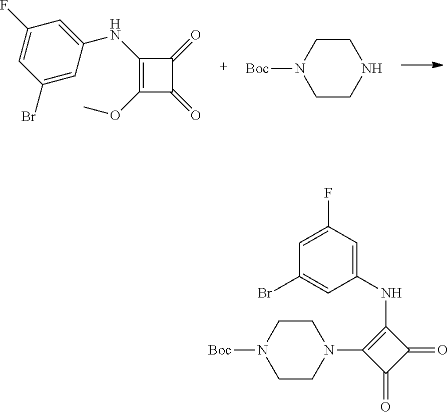 [SMILES: COc1c(Nc2cc(F)cc(Br)c2)c(=O)c1=O ; CC(C)(C)OC(=O)N1CCNCC1 ; CC(C)(C)OC(=O)N1CCN(c2c(Nc3cc(F)cc(Br)c3)c(=O)c2=O)CC1]

3-((3-Bromo-5-fluorophenyl)amino)-4-methoxycyclobut-3-ene-1,2-dione (2.000 g, 6.665 mmol) prepared in Step 1, tert-butyl piperazine-1-carboxylate (1.862 g, 9.997 mmol), and N,N-diisopropylethylamine (2.322 mL, 13.330 mmol) were dissolved in methanol (30 mL) at room temperature, and the reaction solution was stirred for 18 hours at the same temperature. The solvent was removed from the reaction mixture under reduced pressure. The concentrate was purified by column chromatography (SiO₂, 40 g cartridge; ethyl acetate/hexane=0% to 30%) and concentrated to obtain the title compound (2.900 g, 95.8%) as a black foam solid.

### [Step 3] Synthesis of tert-butyl 4-(2-((3-bromo-5-fluorophenyl)(4-(5-(difluoromethyl)-1.3.4-oxadiazol-2-yl)-2-fluorobenzyl)amino)-3,4-dioxocyclobut-1-en-1-yl)piperazine-1-carboxylate

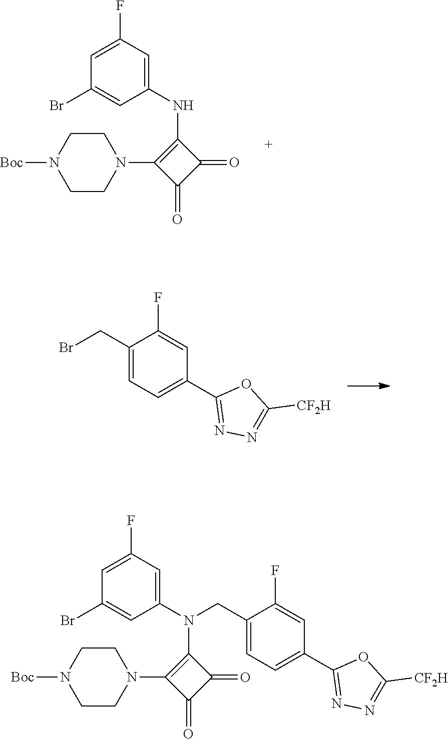 [SMILES: CC(C)(C)OC(=O)N1CCN(c2c(Nc3cc(F)cc(Br)c3)c(=O)c2=O)CC1 ; Fc1cc(-c2nnc(C(F)F)o2)ccc1CBr ; CC(C)(C)OC(=O)N1CCN(c2c(N(Cc3ccc(-c4nnc(C(F)F)o4)cc3F)c3cc(F)cc(Br)c3)c(=O)c2=O)CC1]

Tert-butyl 4-(2-((3-bromo-5-fluorophenyl)amino)-3,4-dioxocyclobut-1-en-1-yl)piperazin-1-carboxylate (2.900 g, 6.383 mmol) prepared in Step 2 was dissolved in N,N-dimethylformamide (30 mL) at 0° C. To the reaction solution, sodium hydride (60.00%, 0.383 g, 9.575 mmol) was added and stirred for 30 minutes at the same temperature. To the reaction mixture, 2-(4-(bromomethyl)-3-fluorophenyl)-5-(difluoromethyl)-1,3,4-oxadiazole (2.548 g, 8.298 mmol) was added and further stirred at room temperature for 3 hours. Water was poured into the reaction mixture, and the mixture was extracted with ethyl acetate. The organic layer was washed with a saturated aqueous sodium chloride solution, dried over anhydrous sodium sulfate, filtered, and concentrated under reduced pressure. The concentrate was purified by column chromatography (SiO₂, 40 g cartridge; ethyl acetate/hexane=0% to 50%) and concentrated to obtain the title compound (2.000 g, 46.0%) as a yellow foam solid.

### [Step 4] Synthesis of 3-((3-bromo-5-fluorophenyl)(4-(5-(difluoromethyl)-1,3,4-oxadiazol-2-yl)-2-fluorobenz yl)amino)-4-(piperazin-1-yl)cyclobut-3-ene-1,2-dione 2,2,2-trifluoroacetate

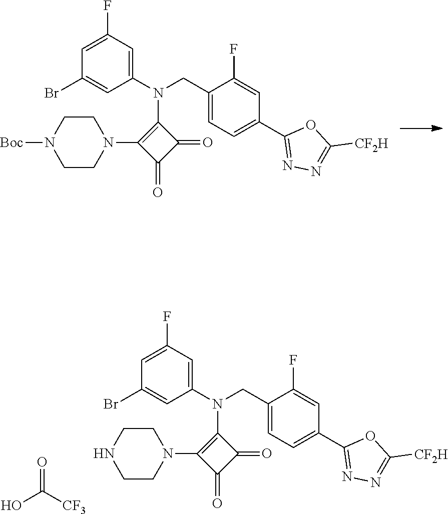 [SMILES: CC(C)(C)OC(=O)N1CCN(c2c(N(Cc3ccc(-c4nnc(C(F)F)o4)cc3F)c3cc(F)cc(Br)c3)c(=O)c2=O)CC1 ; O=C(O)C(F)(F)F ; O=c1c(N2CCNCC2)c(N(Cc2ccc(-c3nnc(C(F)F)o3)cc2F)c2cc(F)cc(Br)c2)c1=O]

Tert-butyl 4-(2-((3-bromo-5-fluorophenyl)(4-(5-(difluoromethyl)-1,3,4-oxadiazol-2-yl)-2-fluorobenzyl)amino)-3,4-dioxocyclobut-1-en-1-yl)piperazine-1-carboxylate (2.000 g, 2.939 mmol) prepared in Step 3 and trifluoroacetic acid (2.251 mL, 29.392 mmol) were dissolved in dichloromethane (20 mL) at room temperature, and the reaction solution was stirred for 18 hours at the same temperature. Water was poured into the reaction mixture, and the mixture was extracted with dichloromethane. The organic layer was washed with a saturated aqueous sodium chloride solution, dried over anhydrous sodium sulfate, filtered, and concentrated under reduced pressure. The obtained product was used without further purification (2.000 g, 98.0%, brown oil).

### [Step 5] Synthesis of Compound 7

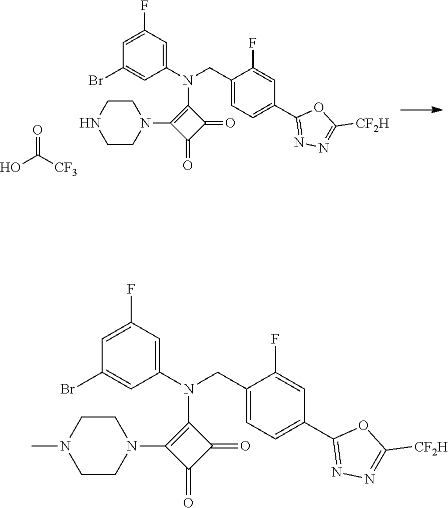 [SMILES: O=C(O)C(F)(F)F ; O=c1c(N2CCNCC2)c(N(Cc2ccc(-c3nnc(C(F)F)o3)cc2F)c2cc(F)cc(Br)c2)c1=O ; CN1CCN(c2c(N(Cc3ccc(-c4nnc(C(F)F)o4)cc3F)c3cc(F)cc(Br)c3)c(=O)c2=O)CC1]

3-((3-Bromo-5-fluorophenyl)(4-(5-(difluoromethyl)-1,3,4-oxadiazol-2-yl)-2-fluorobenzyl)amino)-4-(piperazin-1-yl)cyclobut-3-ene-1,2-dione 2,2,2-trifluoroacetate (2.000 g, 2.880 mmol) prepared in Step 4, N,N-diisopropylethylamine (0.502 mL, 2.880 mmol), formaldehyde (0.173 g, 5.761 mmol), and sodium triacetoxyborohydride (1.221 g, 5.761 mmol) were dissolved in dichloromethane (20 mL) at room temperature. The reaction solution was stirred for 18 hours at the same temperature. Water was poured into the reaction mixture, and the mixture was extracted with dichloromethane. The organic layer was washed with a saturated aqueous sodium chloride solution, dried over anhydrous sodium sulfate, filtered, and concentrated under reduced pressure. The concentrate was purified by column chromatography (SiO₂, 40 g cartridge; methanol/dichloromethane=0% to 10%) and concentrated to obtain the title compound (1.500 g, 87.6%) as a yellow solid.

¹H NMR (400 MHz, CDCl₃) δ 7.92 (dd, J=8.0, 1.5 Hz, 1H), 7.85 (dd, J=10.2, 1.5 Hz, 1H), 7.59 (t, J=7.7 Hz, 1H), 7.06-7.02 (m, 1H), 7.04 (s, 0.25H), 6.93 (s, 0.5H), 6.85 (s, 1H), 6.80 (s, 0.25H), 6.71-6.67 (m, 1H), 5.49 (s, 2H), 3.45 (br s, 4H), 2.39 (br s, 4H), 2.30 (s, 3H); LRMS (ES) m/z 595.4 (M⁺+1).

### Example 8: Synthesis of 3-((4-(5-(difluoromethyl)-1,3,4-oxadiazol-2-yl)-2-fluorobenzyl)(3-fluoro-5-(pyridin-3-yl)phenyl)amino)-4-(4-methylpiperazin-1-yl)cyclobut-3-ene-1,2-dione (Compound 8)

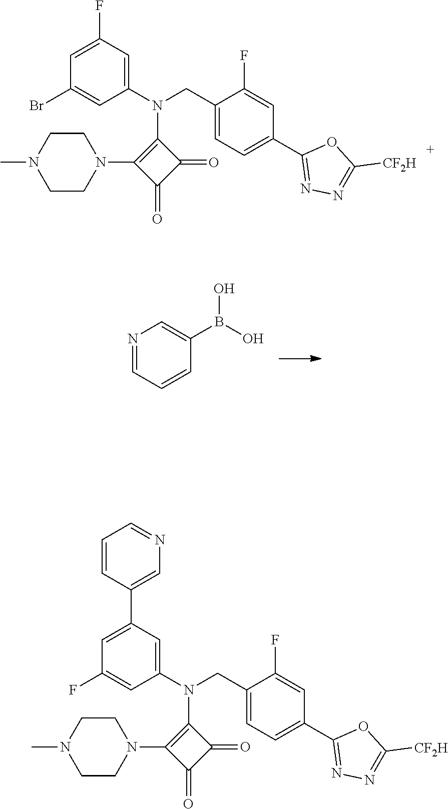 [SMILES: CN1CCN(c2c(N(Cc3ccc(-c4nnc(C(F)F)o4)cc3F)c3cc(F)cc(Br)c3)c(=O)c2=O)CC1 ; OB(O)c1cccnc1 ; CN1CCN(c2c(N(Cc3ccc(-c4nnc(C(F)F)o4)cc3F)c3cc(F)cc(-c4cccnc4)c3)c(=O)c2=O)CC1]

3-((3-Bromo-5-fluorophenyl)(4-(5-(difluoromethyl)-1,3,4-oxadiazol-2-yl)-2-fluorobenzyl)amino)-4-(4-methylpiperazin-1-yl)cyclobut-3-ene-1,2-dione (0.200 g, 0.336 mmol) prepared in Example 7, pyridin-3-ylboronic acid (0.054 g, 0.437 mmol), [1,1′-bis(di-tert-butylphosphino)ferrocene]palladium(II) dichloride (Pd(dtbpf)Cl₂, 0.022 g, 0.034 mmol), and cesium carbonate (0.219 g, 0.673 mmol) were mixed with 1,4-dioxane (9 mL)/water (3 mL) and heated at 100° C. for 20 minutes by microwave irradiation. Then, the reaction was completed by lowering the reaction temperature to room temperature. Water was poured into the reaction mixture, and the mixture was extracted with ethyl acetate. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; methanol/dichloromethane=0% to 20%) and concentrated to obtain the title compound (0.080 g, 40.1%) as a brown oil.

¹H NMR (400 MHz, CDCl₃) (8.78 (d, J=1.9 Hz, 1H), 8.67 (dd, J=4.7, 1.1 Hz, 1H), 7.91 (dd, J=8.0, 1.4 Hz, 1H), 7.85-7.80 (m, 2H), 7.65 (t, J=7.7 Hz, 1H), 7.43 (dd, J=7.9, 4.9 Hz, 1H), 7.11-7.08 (m, 1H), 7.05 (s, 0.25H), 6.96 (s, 1H), 6.92 (s, 0.5H), 6.79 (s, 0.25H), 6.78-6.74 (m, 1H), 5.58 (s, 2H), 3.49 (br s, 4H), 2.28 (br s, 4H), 2.19 (s, 3H); LRMS (ES) m/z 593.5 (M⁺+1).

### Example 9: Synthesis of 3-((4-(5-(difluoromethyl)-1,3,4-oxadiazol-2-yl)-2-fluorobenzyl)(phenyl)amino)-4-(6-(oxetan-3-yl)-2,6-diazaspiro[3.3]heptan-2-yl)cyclobut-3-ene-1,2-dione (Compound 9)

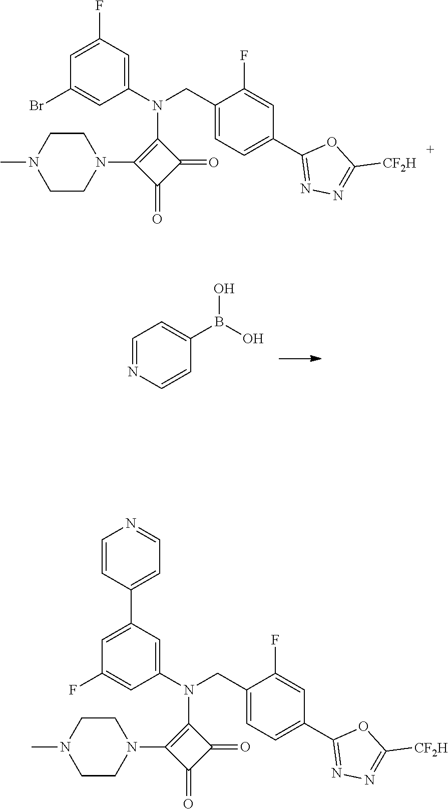 [SMILES: CN1CCN(c2c(N(Cc3ccc(-c4nnc(C(F)F)o4)cc3F)c3cc(F)cc(Br)c3)c(=O)c2=O)CC1 ; OB(O)c1ccncc1 ; CN1CCN(c2c(N(Cc3ccc(-c4nnc(C(F)F)o4)cc3F)c3cc(F)cc(-c4ccncc4)c3)c(=O)c2=O)CC1]

3-((3-Bromo-5-fluorophenyl)(4-(5-(difluoromethyl)-1,3,4-oxadiazol-2-yl)-2-fluorobenzyl)amino)-4-(4-methylpiperazin-1-yl)cyclobut-3-ene-1,2-dione (0.200 g, 0.336 mmol) prepared in Example 7, pyridin-4-ylboronic acid (0.054 g, 0.437 mmol), [1,1′-bis(di-tert-butylphosphino)ferrocene]palladium(II) dichloride (Pd(dtbpf)Cl₂, 0.022 g, 0.034 mmol), and cesium carbonate (0.219 g, 0.673 mmol) were mixed with 1,4-dioxane (9 mL)/water (3 mL) and heated at 100° C. for 20 minutes by microwave irradiation. Then, the reaction was completed by lowering the reaction temperature to room temperature. Water was poured into the reaction mixture, and the mixture was extracted with ethyl acetate. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; methanol/dichloromethane=0% to 20%) and concentrated to obtain the title compound (0.070 g, 35.1%) as a brown oil.

¹H NMR (400 MHz, CDCl₃) δ 8.73 (d, J=5.6 Hz, 1H), 7.92-7.90 (m, 1H), 7.86-7.83 (m, 1H), 7.66 (t, J=7.7 Hz, 1H), 7.43 (d, J=5.9 Hz, 2H), 7.15-7.13 (m, 1H), 7.05 (s, 0.25H), 7.00 (s, 1H), 6.92 (s, 0.5H), 6.81 (s, 0.25H), 6.80-6.78 (m, 1H), 5.58 (s, 2H), 3.30 (br s, 4H), 2.27 (br s, 4H), 2.19 (s, 3H); LRMS (ES) m/z 593.4 (M⁺+1).

### Example 10: Synthesis of 3-((4-(5-(difluoromethyl)-1,3,4-oxadiazol-2-yl)-2-fluorobenzyl)(phenyl)amino)-4-(6-(oxetan-3-yl)-2,6-diazaspiro[3.3]heptan-2-yl)cyclobut-3-ene-1,2-dione (Compound 10)

### [Step 1] Synthesis of tert-butyl 6-(2-methoxy-3,4-dioxocyclobut-1-en-1-yl)-2,6-diazaspiro[3.3]heptane-2-carboxylate

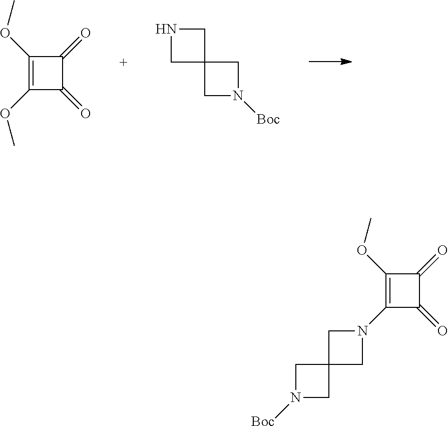

3,4-Dimethoxycyclobut-3-ene-1,2-dione (1.710 g, 12.033 mmol) and tert-butyl 2,6-diazaspiro[3.3]heptane-2-carboxylate (2.386 g, 12.033 mmol) were dissolved in ethanol (30 mL) at room temperature, and the reaction solution was stirred for 18 hours at the same temperature. The precipitated solid was filtered, washed with hexane and dried to obtain the title compound (0.700 g, 18.9%) as a white solid.

### [Step 2] Synthesis of tert-butyl 6-(3,4-dioxo-2-(phenylamino)cyclobut-1-en-1-yl)-2,6-diazaspiro[3.3]heptane-2-carboxylate

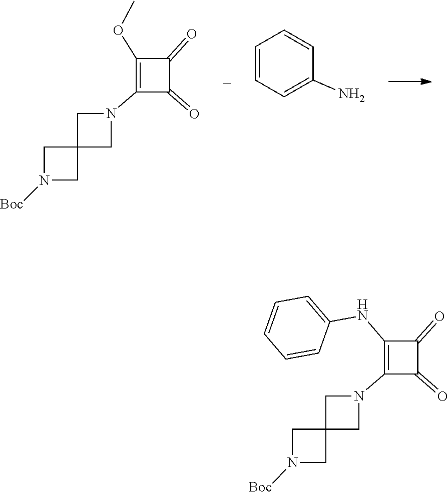 [SMILES: COc1c(N2CC3(CN(C(=O)OC(C)(C)C)C3)C2)c(=O)c1=O ; Nc1ccccc1 ; CC(C)(C)OC(=O)N1CC2(C1)CN(c1c(Nc3ccccc3)c(=O)c1=O)C2]

Tert-butyl 6-(2-methoxy-3,4-dioxocyclobut-1-en-1-yl)-2,6-diazaspiro[3.3]heptane-2-carboxylate (0.700 g, 2.270 mmol) prepared in Step 1, aniline (0.207 mL, 2.270 mmol), and triethylamine (0.633 mL, 4.541 mmol) were dissolved in ethanol (20 mL) at 78° C., and the reaction solution was stirred for 18 hours at the same temperature. Then, the reaction was completed by lowering the reaction temperature to room temperature. The solvent was removed from the reaction mixture under reduced pressure to obtain a concentrate. Into the concentrate, water was poured, and the reaction mixture was extracted with dichloromethane. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; ethyl acetate/hexane=0% to 50%) and concentrated to obtain the title compound (0.600 g, 71.5%) as a white solid.

### [Step 3] Synthesis of tert-butyl 6-(2-((4-(5-(difluoromethyl)-1,3,4-oxadiazol-2-yl)-2-fluorobenzyl)(phenyl)amino)-3,4-dioxocyclobut-1-en-1-yl)-2,6-diazaspiro[3.3]heptane-2-carboxylate

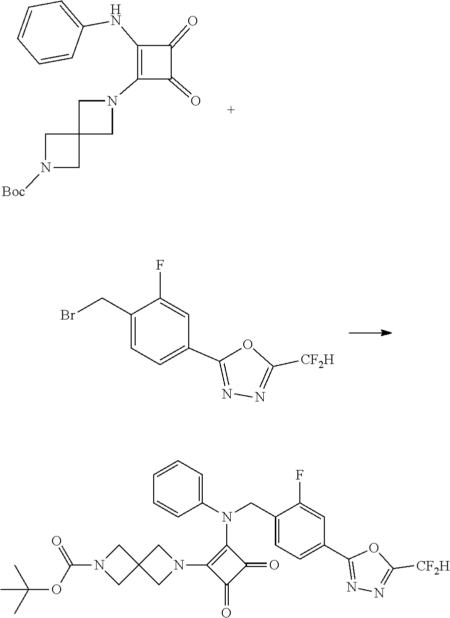 [SMILES: CC(C)(C)OC(=O)N1CC2(C1)CN(c1c(Nc3ccccc3)c(=O)c1=O)C2 ; Fc1cc(-c2nnc(C(F)F)o2)ccc1CBr ; CC(C)(C)OC(=O)N1CC2(C1)CN(c1c(N(Cc3ccc(-c4nnc(C(F)F)o4)cc3F)c3ccccc3)c(=O)c1=O)C2]

Tert-butyl 6-(3,4-dioxo-2-(phenylamino)cyclobut-1-en-1-yl)-2,6-diazaspiro[3.3]heptane-2-carboxylate (0.687 g, 1.860 mmol) prepared in Step 2 was dissolved in N,N-dimethylformamide (5 mL) at 0° C. To the reaction solution, sodium hydride (60.00%, 0.112 g, 2.790 mmol) was added and stirred for 30 minutes at the same temperature. To the reaction mixture, 2-(4-(bromomethyl)-3-fluorophenyl)-5-(difluoromethyl)-1,3,4-oxadiazole (0.685 g, 2.232 mmol) was added and further stirred at room temperature for 3 hours. Water was poured into the reaction mixture, and the mixture was extracted with ethyl acetate. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; ethyl acetate/hexane=0% to 50%) and concentrated to obtain the title compound (0.700 g, 63.2%) as a white solid.

### [Step 4] Synthesis of 3-((4-(5-(difluoromethyl)-1,3,4-oxadiazol-2-yl)-2-fluorobenzyl)(phenyl)amino)-4-(2,6-diazaspiro[3.3]heptan-2-yl)cyclobut-3-ene-1,2-dione

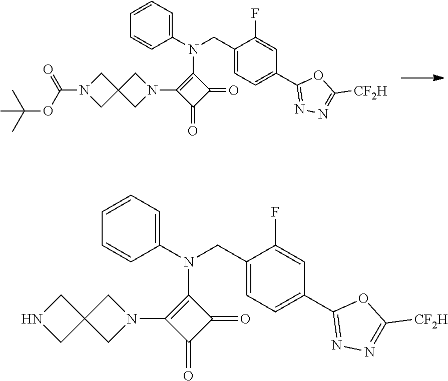 [SMILES: CC(C)(C)OC(=O)N1CC2(C1)CN(c1c(N(Cc3ccc(-c4nnc(C(F)F)o4)cc3F)c3ccccc3)c(=O)c1=O)C2 ; O=c1c(N2CC3(CNC3)C2)c(N(Cc2ccc(-c3nnc(C(F)F)o3)cc2F)c2ccccc2)c1=O]

Tert-butyl 6-(2-((4-(5-(difluoromethyl)-1,3,4-oxadiazol-2-yl)-2-fluorobenzyl)(phenyl)amino)-3,4-dioxocyclobut-1-en-1-yl)-2,6-diazaspiro[3.3]heptane-2-carboxylate (0.700 g, 1.175 mmol) prepared in Step 3 and trifluoroacetic acid (0.900 mL, 11.753 mmol) were dissolved in dichloromethane (10 mL) at room temperature, and the reaction solution was stirred for 18 hours at the same temperature. A saturated aqueous solution of sodium hydrogen carbonate was poured into the reaction mixture, followed by extraction with dichloromethane. The organic layer was washed with a saturated aqueous sodium chloride solution, dried over anhydrous sodium sulfate, filtered, and concentrated under reduced pressure. The obtained product was used without further purification (0.400 g, 68.7%, white solid).

### [Step 5] Synthesis of Compound 10

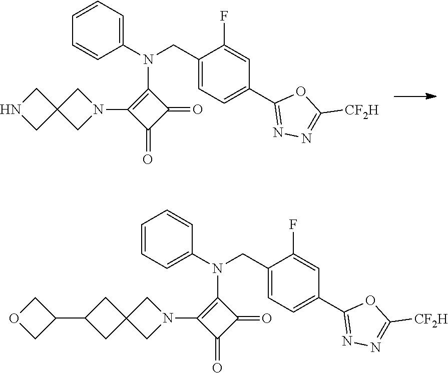 [SMILES: O=c1c(N2CC3(CNC3)C2)c(N(Cc2ccc(-c3nnc(C(F)F)o3)cc2F)c2ccccc2)c1=O ; O=c1c(N2CC3(CC(C4COC4)C3)C2)c(N(Cc2ccc(-c3nnc(C(F)F)o3)cc2F)c2ccccc2)c1=O]

3-((4-(5-(Difluoromethyl)-1,3,4-oxadiazol-2-yl)-2-fluorobenzyl)(phenyl)amino)-4-(2, 6-diazaspiro[3.3]heptan-2-yl)cyclobut-3-ene-1,2-dione (0.100 g, 0.202 mmol) prepared in Step 4, 3-oxetanone (0.029 g, 0.404 mmol), and sodium triacetoxyborohydride (0.086 g, 0.404 mmol) were dissolved in dichloromethane (10 mL) at room temperature. The reaction solution was stirred for 18 hours at the same temperature. Water was poured into the reaction mixture, and the mixture was extracted with dichloromethane. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; methanol/dichloromethane=0% to 20%) and concentrated to obtain the title compound (0.080 g, 71.9%) as a colorless oil.

¹H NMR (400 MHz, CDCl₃) δ 7.84 (dd, J=8.0, 1.4 Hz, 1H), 7.69 (dd, J=9.8, 1.4 Hz, 1H), 7.61 (t, J=7.6 Hz, 1H), 7.39-7.33 (m, 3H), 7.06-7.04 (m, 2H), 7.06 (s, 0.25H), 6.91 (s, 0.5H), 6.78 (s, 0.25H), 5.44 (s, 2H), 4.62-4.58 (m, 2H), 4.34-4.31 (m, 2H), 3.80 (br s, 4H), 3.60-3.58 (m, 1H), 3.23 (s, 4H); LRMS (ES) m/z 552.5 (M⁺+1).

### Example 11: Synthesis of 3-((4-(5-(difluoromethyl)-1,3,4-oxadiazol-2-yl)benzyl)(phenyl)amino)-4-(6-methyl-2,6-diazaspyro[3.3]heptan-2-yl)cyclobut-3-ene-1,2-dione (Compound 11)

### [Step 1] Synthesis of tert-butyl 6-(2-((4-(5-(difluoromethyl)-1,3,4-oxadiazol-2-yl benzyl)(phenyl)amino)-3,4-dioxocyclobut-1-en-1-yl)-2,6-diazaspiro[3.3]heptane-2-carboxylate

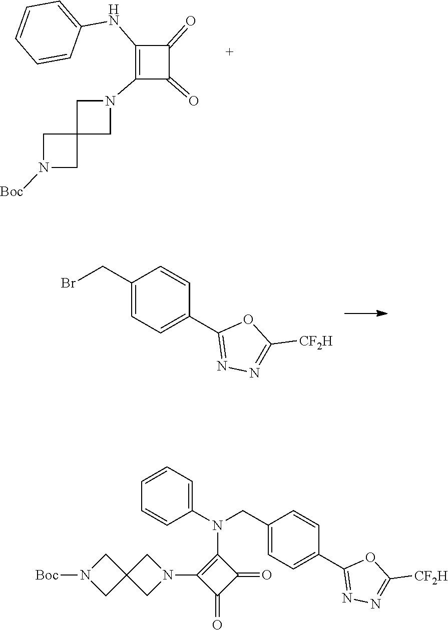 [SMILES: CC(C)(C)OC(=O)N1CC2(C1)CN(c1c(Nc3ccccc3)c(=O)c1=O)C2 ; FC(F)c1nnc(-c2ccc(CBr)cc2)o1 ; CC(C)(C)OC(=O)N1CC2(C1)CN(c1c(N(Cc3ccc(-c4nnc(C(F)F)o4)cc3)c3ccccc3)c(=O)c1=O)C2]

Tert-butyl 6-(3,4-dioxo-2-(phenylamino)cyclobut-1-en-1-yl)-2,6-diazaspiro[3.3]heptane-2-carboxylate (0.177 g, 0.479 mmol) prepared in Step 2 of Example 10 was dissolved in N,N-dimethylformamide (10 mL) at 0° C. To the reaction solution, sodium hydride (60.00%, 0.029 g, 0.719 mmol) was added and stirred for 30 minutes at the same temperature. To the reaction mixture, 2-(4-(bromomethyl)phenyl)-5-(difluoromethyl)-1,3,4-oxadiazole (0.166 g, 0.575 mmol) was added and further stirred at room temperature for 3 hours. Water was poured into the reaction mixture, and the mixture was extracted with ethyl acetate. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; ethyl acetate/hexane=0% to 50%) and concentrated to obtain the title compound (0.150 g, 54.2%) as a white solid.

### [Step 2] Synthesis of 3-((4-(5-(difluoromethyl)-1.3.4-oxadiazol-2-yl)benzyl)(phenyl)amino)-4-(2.6-diazaspiro[3.3]heptan-2-yl)cyclobut-3-ene-1.2-dione 2.2.2-trifluoroacetate

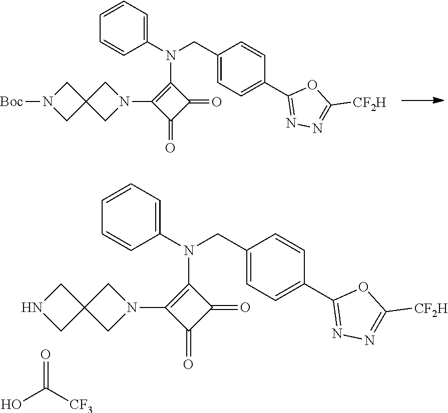

Tert-butyl 6-(2-((4-(5-(difluoromethyl)-1,3,4-oxadiazol-2-yl)benzyl)(phenyl)amino)-3,4-dioxocyclobut-1-en-1-yl)-2,6-diazaspiro[3.3]heptane-2-carboxylate (0.180 g, 0.312 mmol) prepared in Step 1 and trifluoroacetic acid (0.239 mL, 3.116 mmol) were dissolved in dichloromethane (10 mL) at room temperature, and the reaction solution was stirred for 18 hours at the same temperature. After removing the solvent from the reaction mixture under reduced pressure, the obtained product was used without further purification (0.180 g, 97.6%, yellow oil).

### [Step 3] Synthesis of Compound 11

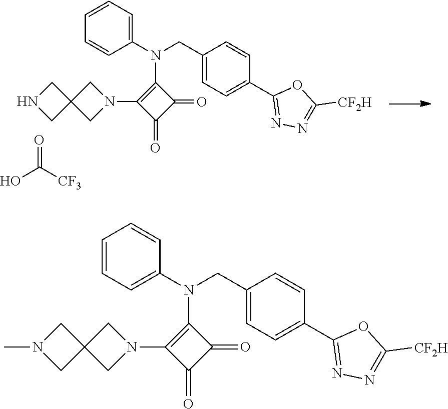

3-((4-(5-(Difluoromethyl)-1,3,4-oxadiazol-2-yl)benzyl)(phenyl)amino)-4-(2,6-diazaspiro[3.3]heptan-2-yl)cyclobut-3-ene-1,2-dione 2,2,2-trifluoroacetate (0.180 g, 0.304 mmol) prepared in Step 2, N,N-diisopropylethylamine (0.053 mL, 0.304 mmol), formaldehyde (0.018 g, 0.609 mmol), and sodium triacetoxyborohydride (0.129 g, 0.609 mmol) were dissolved in dichloromethane (10 mL) at room temperature. The reaction solution was stirred for 18 hours at the same temperature. Water was poured into the reaction mixture, and the mixture was extracted with dichloromethane. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; methanol/dichloromethane=0% to 20%) and concentrated to obtain the title compound (0.056 g, 37.4%) as a white solid.

¹H NMR (400 MHz, CDCl₃) δ 8.02 (d, J=8.2 Hz, 2H), 7.40-7.35 (m, 5H), 7.04 (s, 0.25H), 7.03-7.01 (m, 2H), 6.91 (s, 0.5H), 6.78 (s, 0.25H), 5.33 (s, 2H), 3.68 (s, 4H), 2.45 (s, 3H), 1.45 (s, 4H); LRMS (ES) m/z 492.4 (M⁺+1).

### Example 12: Synthesis of 3-((3-bromophenyl)(4-(5-(difluoromethyl)-1,3,4-oxadiazol-2-yl)-2-fluorobenzyl)amino)-4-(4-methylpiperazin-1-yl)cyclobut-3-ene-1,2-dione (Compound 12)

### [Step 1] Synthesis of 3-((3-bromophenyl)amino)-4-methoxycyclobut-3-ene-1.2-dione

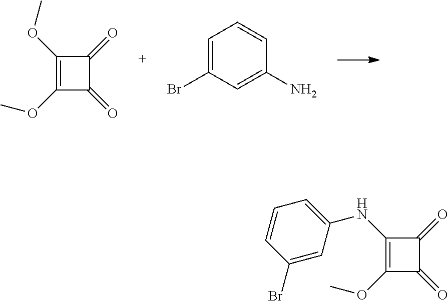

3,4-Dimethoxycyclobut-3-ene-1,2-dione (2.000 g, 14.074 mmol) and 3-bromoaniline (2.421 g, 14.074 mmol) were dissolved in methanol (50 mL) at room temperature and stirred for 18 hours at the same temperature. The precipitated solid was filtered, washed with hexane and dried to obtain the title compound (3.500 g, 88.2%) as a white solid.

### [Step 2] Synthesis of tert-butyl 4-(2-((3-bromophenyl)amino)-3.4-dioxocyclobut-1-en-1-yl)piperazine-1-carboxylate

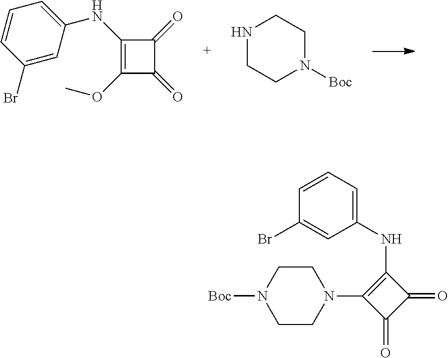 [SMILES: COc1c(Nc2cccc(Br)c2)c(=O)c1=O ; CC(C)(C)OC(=O)N1CCNCC1 ; CC(C)(C)OC(=O)N1CCN(c2c(Nc3cccc(Br)c3)c(=O)c2=O)CC1]

3-((3-Bromophenyl)amino)-4-methoxycyclobut-3-ene-1,2-dione (1.250 g, 4.431 mmol) prepared in Step 1, tert-butylpiperazine-1-carboxylate (1.238 g, 6.647 mmol), and N,N-diisopropylethylamine (1.544 mL, 8.862 mmol) were dissolved in ethanol (20 mL) at 78° C., and the reaction solution was stirred at the same temperature for 18 hours. Then, the reaction was completed by lowering the reaction temperature to room temperature. The precipitated solid was filtered, washed with hexane and dried to obtain the title compound (1.200 g, 62.1%) as a white solid.

### [Step 3] Synthesis of tert-butyl 4-(2-((3-bromophenyl)(4-(5-(difluoromethyl)-1.3.4-oxadiazol-2-yl)-2-fluorobenzyl)amino)-3,4-dioxocyclobut-1-en-1-yl)piperazine-1-carboxylate

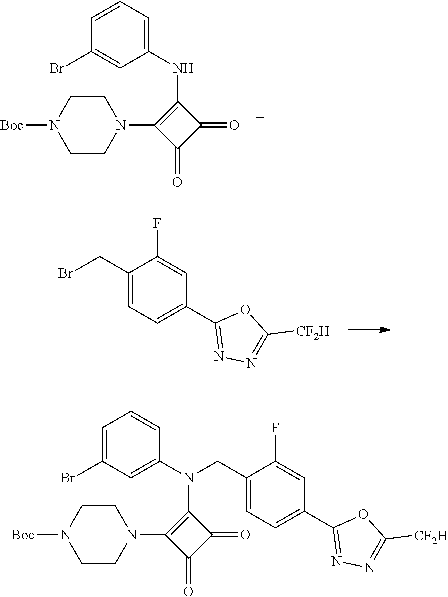

Tert-butyl 4-(2-((3-bromophenyl)amino)-3,4-dioxocyclobut-1-en-1-yl)piperazin-1-carboxylate (1.200 g, 2.750 mmol) prepared in Step 2 was dissolved in N,N-dimethylformamide (30 mL) at 0° C. To the reaction solution, sodium hydride (60.00%, 0.143 g, 3.575 mmol) was added and stirred for 30 minutes at the same temperature. To the reaction mixture, 2-(4-(bromomethyl)-3-fluorophenyl)-5-(difluoromethyl)-1,3,4-oxadiazole (1.013 g, 3.300 mmol) was added and further stirred for 3 hours at room temperature. Water was poured into the reaction mixture, and the mixture was extracted with ethyl acetate. The organic layer was washed with a saturated aqueous sodium chloride solution, dried over anhydrous sodium sulfate, filtered, and concentrated under reduced pressure. The concentrate was purified by column chromatography (SiO₂, 40 g cartridge; ethyl acetate/hexane=0% to 50%) and concentrated to obtain the title compound (1.400 g, 76.8%) as a colorless oil.

### [Step 4] Synthesis of 3-((3-bromophenyl)(4-(5-(difluoromethyl)-1,3,4-oxadiazol-2-yl)-2-fluorobenzyl)amino)-4-(piperazin-1-yl)cyclobut-3-ene-1,2-dione 2,2,2-trifluoroacetate

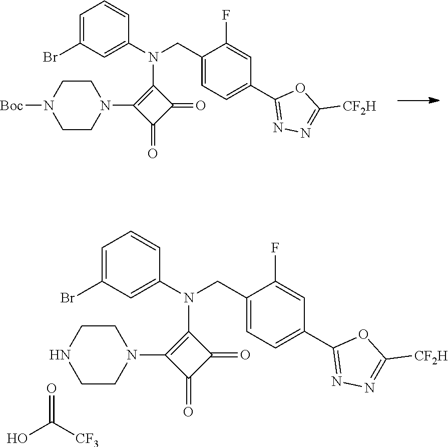

Tert-butyl 4-(2-((3-bromophenyl)(4-(5-(difluoromethyl)-1,3,4-oxadiazol-2-yl)-2-fluorobenzyl)amino)-3,4-dioxocyclobut-1-en-1-yl)piperazine-1-carboxylate (1.400 g, 2.113 mmol) prepared in step 3 and trifluoroacetic acid (1.618 mL, 21.13 mmol) were dissolved in dichloromethane (20 mL) at room temperature, and the reaction solution was stirred for 18 hours at the same temperature. After removing the solvent from the reaction mixture under reduced pressure, the obtained product was used without further purification (1.400 g, 97.9%, brown oil).

### [Step 5] Synthesis of Compound 12

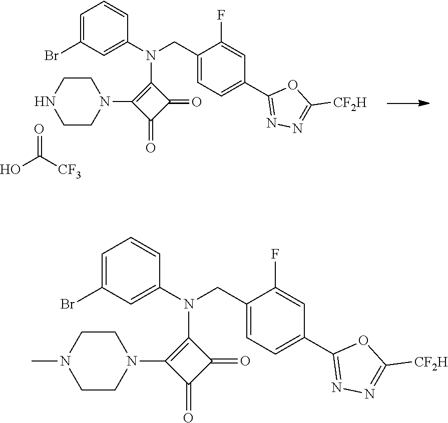

3-((3-Bromophenyl)(4-(5-(difluoromethyl)-1,3,4-oxadiazol-2-yl)-2-fluorobenzyl)amino)-4-(piperazin-1-yl)cyclobut-3-ene-1,2-dione 2,2,2-trifluoroacetate (1.400 g, 2.070 mmol) prepared in Step 4, formaldehyde (0.124 g, 4.140 mmol), N,N-diisopropylethylamine (0.361 mL, 2.070 mmol), and sodium triacetoxyborohydride (0.877 g, 4.140 mmol) were dissolved in dichloromethane (10 mL) at room temperature. The reaction solution was stirred for 18 hours at the same temperature. Water was poured into the reaction mixture, and the mixture was extracted with dichloromethane. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; methanol/dichloromethane=0% to 10%) and concentrated to obtain the title compound (0.900 g, 75.4%) as a white solid.

¹H NMR (400 MHz, CDCl₃) δ 7.89 (dd, J=8.0, 1.5 Hz, 1H), 7.80 (dd, J=10.1, 1.5 Hz, 1H), 7.60 (t, J=7.6 Hz, 1H), 7.30-7.28 (m, 1H), 7.23 (t, J=8.0 Hz, 1H), 7.08 (t, J=1.9 Hz, 1H), 7.05 (s, 0.25H), 6.98 (dd, J=8.0, 1.5 Hz, 1H), 6.92 (s, 0.5H), 6.79 (s, 0.25H), 5.53 (s, 2H), 3.36 (br s, 4H), 2.45 (br s, 4H), 2.25 (s, 3H); LRMS (ES) m/z 577.4 (M⁺+1).

### Example 13: Synthesis of 3-((4-(5-(difluoromethyl)-1,3,4-oxadiazol-2-yl)-2-fluorobenzyl)(phenyl)amino)-4-((1S,4S)-5-(oxetan-3-yl)-2,5-diazabicyclo[2.2.1]heptan-2-yl)cyclobut-3-ene-1,2-dione (Compound 13)

### [Step 1] Synthesis of tert-butyl (1S,4S)-5-(3,4-dioxo-2-(phenylamino)cyclobut-1-en-1-yl)-2,5-diazabicyclo[2.2.1]heptane-2-carboxylate

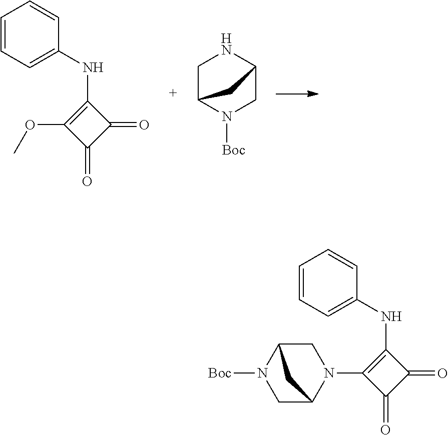 [SMILES: COc1c(Nc2ccccc2)c(=O)c1=O ; CC(C)(C)OC(=O)N1C[C@@H]2C[C@H]1CN2 ; CC(C)(C)OC(=O)N1C[C@@H]2C[C@H]1CN2c1c(Nc2ccccc2)c(=O)c1=O]

3-Methoxy-4-(phenylamino)cyclobut-3-ene-1,2-dione (0.500 g, 2.461 mmol), tertbutyl (1S,4S)-2,5-diazabicyclo[2.2.1]heptane-2-carboxylate (0.537 g, 2.707 mmol), and N,N-diisopropylethylamine (0.85 mL, 4.921 mmol) were dissolved in methanol (10 mL) at room temperature, and the reaction solution was stirred for 18 hours at room temperature. After removing the solvent from the reaction mixture under reduced pressure, the concentrate was purified by column chromatography (SiO₂, 12 g cartridge; ethyl acetate/hexane=20% to 40%) and concentrated to obtain the title compound (0.871 g, 95.8%) as a pale yellow solid.

### [Step 2] Synthesis of tert-butyl (1S,4S)-5-(2-((4-(5-(difluoromethyl)-1,3,4-oxadiazol-2-yl)-2-fluorobenzyl)(phenyl)amino)-3,4-dioxocyclobut-1-en-1-yl)-2,5-diazabicyclo[2.2.1]heptane-2-carboxylate

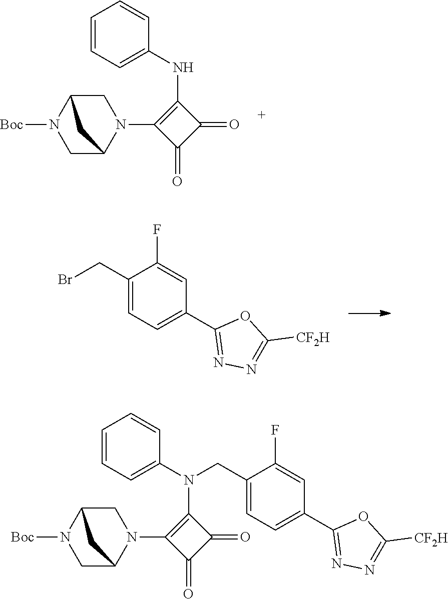 [SMILES: CC(C)(C)OC(=O)N1C[C@@H]2C[C@H]1CN2c1c(Nc2ccccc2)c(=O)c1=O ; Fc1cc(-c2nnc(C(F)F)o2)ccc1CBr ; CC(C)(C)OC(=O)N1C[C@@H]2C[C@H]1CN2c1c(N(Cc2ccc(-c3nnc(C(F)F)o3)cc2F)c2ccccc2)c(=O)c1=O]

Tert-butyl (1S,4S)-5-(3,4-dioxo-2-(phenylamino)cyclobut-1-en-1-yl)-2,5-diazabicyclo[2.2.1]heptane-2-carboxylate (0.300 g, 0.812 mmol) prepared in Step 1, 2-(4-(bromomethyl)-3-fluorophenyl)-5-(difluoromethyl)-1,3,4-oxadiazole (0.274 g, 0.893 mmol), potassium carbonate (0.224 g, 1.624 mmol), and potassium iodide (0.013 g, 0.081 mmol) were dissolved in N,N-dimethylformamide (4 mL) at room temperature, and the reaction solution was stirred for 18 hours at 60° C. Then, the reaction was completed by lowering the reaction temperature to room temperature. The solvent was removed from the reaction mixture under reduced pressure to obtain a concentrate. Into the concentrate, a saturated aqueous solution of sodium hydrogen carbonate was poured, and the reaction mixture was extracted with dichloromethane. The mixture was filtered through a plastic filter to remove the solid residue and the aqueous layer and then concentrated under reduced pressure. The concentrate was purified by column chromatography (SiO₂, 12 g cartridge; ethyl acetate/hexane=30% to 60%) and concentrated to obtain the title compound (0.315 g, 65.1%) as a yellow solid.

### [Step 3] Synthesis of 3-((1S,4S)-2,5-diazabicyclo[2.2.1]heptan-2-yl)-4-((4-(5-(difluoromethyl)-1,3,4-oxadiazol-2-yl-2-fluorobenzyl)(phenyl)amino)cyclobut-3-ene-1.2-dione

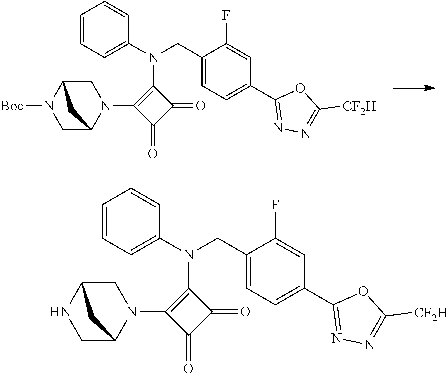

Tert-butyl (1S,4S)-5-(2-((4-(5-(difluoromethyl)-1,3,4-oxadiazol-2-yl)-2-fluorobenzyl)(phenyl)amino)-3,4-dioxocyclobut-1-en-1-yl)-2,5-diazabicyclo[2.2.1]heptane-2-carboxylate (0.315 g, 0.529 mmol) prepared in Step 2 and trifluoroacetic acid (0.284 mL, 3.702 mmol) were dissolved in dichloromethane (4 mL) at room temperature, and the reaction solution was stirred for 18 hours at the same temperature. A saturated aqueous solution of sodium hydrogen carbonate was poured into the reaction mixture, followed by extraction with dichloromethane. The mixture was filtered through a plastic filter to remove the solid residue and the aqueous layer and then concentrated under reduced pressure. The obtained product was used without further purification (0.221 g, 84.3%, pale yellow solid).

### [Step 4] Synthesis of Compound 13

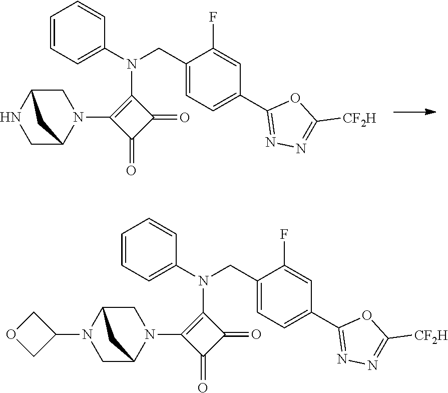 [SMILES: O=c1c(N(Cc2ccc(-c3nnc(C(F)F)o3)cc2F)c2ccccc2)c(N2C[C@@H]3C[C@H]2CN3)c1=O ; O=c1c(N(Cc2ccc(-c3nnc(C(F)F)o3)cc2F)c2ccccc2)c(N2C[C@@H]3C[C@H]2CN3C2COC2)c1=O]

3-((1S,4S)-2,5-diazabicyclo[2.2.1]heptan-2-yl)-4-((4-(5-(difluoromethyl)-1,3,4-oxadiazol-2-yl)-2-fluorobenzyl)(phenyl)amino)cyclobut-3-ene-1,2-dione (0.100 g, 0.202 mmol) prepared in Step 3 and formaldehyde (0.018 mL, 0.303 mmol) were dissolved in dichloromethane (4 mL) at room temperature. To the reaction solution, sodium triacetoxyborohydride (0.086 g, 0.404 mmol) was added and stirred for 18 hours at the same temperature. A saturated aqueous solution of sodium hydrogen carbonate was poured into the reaction mixture, followed by extraction with dichloromethane. The mixture was filtered through a plastic filter to remove the solid residue and the aqueous layer and then concentrated under reduced pressure. The concentrate was purified by column chromatography (SiO₂, 4 g cartridge; methanol/dichloromethane=0% to 2.5%) and concentrated to obtain the title compound (0.075 g, 67.4%) as a yellow solid.

¹H NMR (400 MHz, CDCl₃) δ 7.87 (dd, J=8.0, 1.4 Hz, 1H), 7.72-7.66 (m, 2H), 7.35 (t, J=7.6 Hz, 2H), 7.29-7.25 (m, 1H), 7.07 (d, J=7.6 Hz, 2H), 7.05-6.79 (m, 1H), 5.56 (d, J=15.4 Hz, 1H), 5.47 (d, J=15.4 Hz, 1H), 4.63-4.57 (m, 2H), 4.40 (t, J=5.9 Hz, 2H), 3.74-3.71 (m, 1H), 3.13 (brs, 1H), 2.78 (brs, 1H), 2.67 (brs, 1H), 1.95 (brs, 1H), 1.76 (d, J=9.8 Hz, 1H), 1.54 (d, J=10.1 Hz, 1H), 1.30-1.28 (m, 1H); LRMS (ES) m/z 552.8 (M⁺+1).

### Example 14: Synthesis of 3-((4-(5-(difluoromethyl)-1,3,4-oxadiazol-2-yl)-2-fluorobenzyl)(phenyl)amino)-4-((1S,4S)-5-methyl-2,5-diazabicyclo[2.2.1]heptan-2-yl)cyclobut-3-ene-1,2-dione (Compound 14)

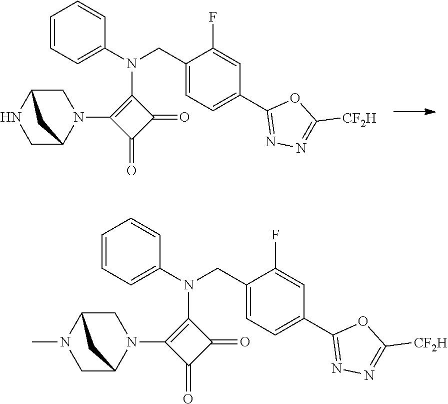 [SMILES: O=c1c(N(Cc2ccc(-c3nnc(C(F)F)o3)cc2F)c2ccccc2)c(N2C[C@@H]3C[C@H]2CN3)c1=O ; CN1C[C@@H]2C[C@H]1CN2c1c(N(Cc2ccc(-c3nnc(C(F)F)o3)cc2F)c2ccccc2)c(=O)c1=O]

3-((1S,4S)-2,5-diazabicyclo[2.2.1]heptan-2-yl)-4-((4-(5-(difluoromethyl)-1,3,4-oxadiazol-2-yl)-2-fluorobenzyl)(phenyl)amino)cyclobut-3-ene-1,2-dione (0.100 g, 0.202 mmol) prepared in Step 3 of Example 14 and formaldehyde (38.00% solution, 0.022 mL, 0.303 mmol) were dissolved in dichloromethane (4 mL) at room temperature. To the reaction solution, sodium triacetoxyborohydride (0.086 g, 0.404 mmol) was added and stirred for 18 hours at the same temperature. A saturated aqueous solution of sodium hydrogen carbonate was poured into the reaction mixture, followed by extraction with dichloromethane. The mixture was filtered through a plastic filter to remove the solid residue and the aqueous layer and then concentrated under reduced pressure. The concentrate was purified by column chromatography (SiO₂, 4 g cartridge; methanol/dichloromethane=0% to 5%) and concentrated to obtain the title compound (0.049 g, 47.7%) as a yellow solid.

¹H NMR (400 MHz, CDCl₃) δ 7.88 (dd, J=8.0, 1.5 Hz, 1H), 7.74-7.67 (m, 2H), 7.35 (t, J=7.6 Hz, 2H), 7.28-7.25 (m, 1H), 7.07-6.79 (m, 3H), 5.58-5.47 (m, 2H), 3.13 (brs, 1H), 2.71 (brs, 1H), 2.63 (brs, 1H), 2.28 (s, 3H), 1.99-1.83 (m, 3H), 1.52 (d, J=10.1 Hz, 1H), 1.31-1.29 (m, 1H); LRMS (ES) m/z 510.8 (M⁺+1).

### Example 15: Synthesis of 3-(((5-(5-(difluoromethyl)-1,3,4-oxadiazol-2-yl)pyridin-2-yl)methyl)(phenyl)amino)-4-((1S,4S)-5-methyl-2,5-diazabicyclo[2.2.1]heptan-2-yl)cyclobut-3-ene-1,2-dione (Compound 15)

### [Step 1] Synthesis of tert-butyl (1S,4S)-5-(2-(((5-(5-(difluoromethyl)-1,3,4-oxadiazol-2-yl)pyridin-2-yl)methyl)(phenyl)amino)-3,4-dioxocyclobut-1-en-1-yl)-2,5-diazabicyclo[2.2.1]heptane-2-carboxylate

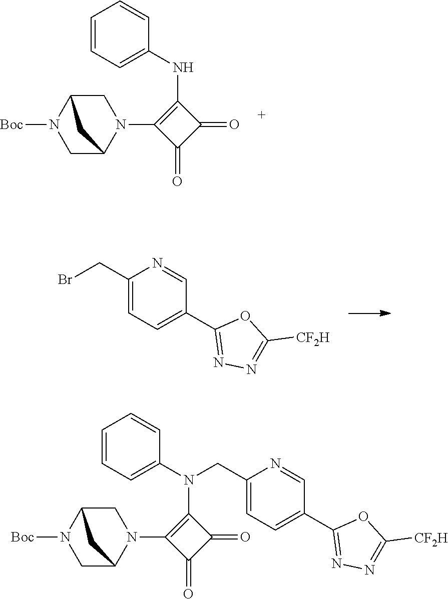 [SMILES: CC(C)(C)OC(=O)N1C[C@@H]2C[C@H]1CN2c1c(Nc2ccccc2)c(=O)c1=O ; FC(F)c1nnc(-c2ccc(CBr)nc2)o1 ; CC(C)(C)OC(=O)N1C[C@@H]2C[C@H]1CN2c1c(N(Cc2ccc(-c3nnc(C(F)F)o3)cn2)c2ccccc2)c(=O)c1=O]

Tert-butyl (1S,4S)-5-(3,4-dioxo-2-(phenylamino)cyclobut-1-en-1-yl)-2,5-diazabicyclo[2.2.1]heptane-2-carboxylate (0.500 g, 1.353 mmol) prepared in Step 1 of Example 13, 2-(6-(bromomethyl)pyridin-3-yl)-5-(difluoromethyl)-1,3,4-oxadiazole (0.432 g, 1.489 mmol), potassium carbonate (0.281 g, 2.030 mmol), and potassium iodide (0.022 g, 0.135 mmol) were dissolved in N,N-dimethylformamide (4 mL) at room temperature, and the reaction solution was stirred for 18 hours at 60° C. Then, the reaction was completed by lowering the reaction temperature to room temperature. The solvent was removed from the reaction mixture under reduced pressure to obtain a concentrate. Into the concentrate, a saturated aqueous solution of sodium hydrogen carbonate was poured, and the reaction mixture was extracted with dichloromethane. The mixture was filtered through a plastic filter to remove the solid residue and the aqueous layer and then concentrated under reduced pressure. The concentrate was purified by column chromatography (SiO₂, 12 g cartridge; ethyl acetate/hexane=30% to 65%) and concentrated to obtain the title compound (0.440 g, 56.2%) as a pale brown solid.

### [Step 2] Synthesis of 3-((1S,4S)-2,5-diazabicyclo[2.2.1]heptan-2-yl)-4-(((5-(5-(difluoromethyl)-1,3,4-oxadiazol-2-yl)pyridin-2-yl)methyl)(phenyl)amino)cyclobut-3-ene-1,2-dione

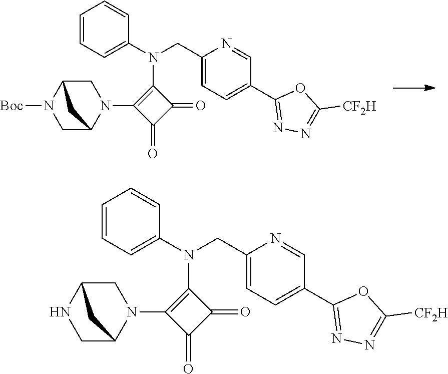

Tert-butyl (1S,4S)-5-(2-(((5-(5-(difluoromethyl)-1,3,4-oxadiazol-2-yl)pyridin-2-yl)methyl)(phenyl)amino)-3,4-dioxocyclobut-1-en-1-yl)-2,5-diazabicyclo[2.2.1]heptane-2-carboxylate (0.440 g, 0.760 mmol) prepared in Step 1 and trifluoroacetic acid (0.291 mL, 3.802 mmol) were dissolved in dichloromethane (4 mL) at room temperature, and the reaction solution was stirred for 18 hours at the same temperature. A saturated aqueous solution of sodium hydrogen carbonate was poured into the reaction mixture, followed by extraction with dichloromethane. The mixture was filtered through a plastic filter to remove the solid residue and the aqueous layer and then concentrated under reduced pressure. The obtained product was used without further purification (0.277 g, 76.1%, brown solid).

### [Step 3] Synthesis of Compound 15

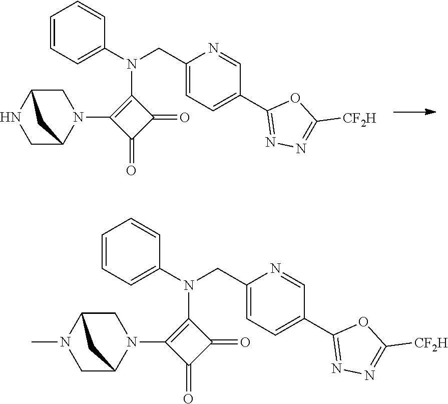 [SMILES: O=c1c(N(Cc2ccc(-c3nnc(C(F)F)o3)cn2)c2ccccc2)c(N2C[C@@H]3C[C@H]2CN3)c1=O ; CN1C[C@@H]2C[C@H]1CN2c1c(N(Cc2ccc(-c3nnc(C(F)F)o3)cn2)c2ccccc2)c(=O)c1=O]

3-((1S,4S)-2,5-diazabicyclo[2.2.1]heptan-2-yl)-4-(((5-(5-(difluoromethyl)-1,3,4-oxadiazol-2-yl)pyridin-2-yl)methyl)(phenyl)amino)cyclobut-3-ene-1,2-dione (0.090 g, 0.188 mmol) prepared in Step 2 and formaldehyde (38.00% solution in water, 0.021 mL, 0.282 mmol) were dissolved in dichloromethane (4 mL) at room temperature. To the reaction solution, sodium triacetoxyborohydride (0.080 g, 0.376 mmol) was added and stirred for 18 hours at the same temperature. A saturated aqueous solution of sodium hydrogen carbonate was poured into the reaction mixture, followed by extraction with dichloromethane. The mixture was filtered through a plastic filter to remove the solid residue and the aqueous layer and then concentrated under reduced pressure. The concentrate was purified by column chromatography (SiO₂, 4 g cartridge; methanol/dichloromethane=0% to 5%) and concentrated to obtain the title compound (0.054 g, 58.3%) as an orange solid.

¹H NMR (400 MHz, CDCl₃) δ 9.25 (d, J=2.0 Hz, 1H), 8.37 (dd, J=8.2, 2.2 Hz, 1H), 7.64 (d, J=8.2 Hz, 1H), 7.36 (t, J=7.8 Hz, 2H), 7.24 (t, J=7.4 Hz, 1H), 7.19 (d, J=7.6 Hz, 2H), 7.08-6.82 (m, 1H), 5.63-5.50 (m, 2H), 3.18 (brs, 1H), 2.76 (brs, 1H), 2.63 (brs, 1H), 2.31 (s, 3H), 2.16-2.14 (m, 2H), 1.87 (d, J=9.9 Hz, 1H), 1.57-1.53 (m, 1H), 1.32-1.28 (m, 1H); LRMS (ES) m/z 493.8 (M⁺+1).

### Example 16: Synthesis of 3-(((5-(5-(difluoromethyl)-1,3,4-oxadiazol-2-yl)pyridin-2-yl)methyl)(phenyl)amino)-4-((1S,4S)-5-isopropyl-2,5-diazabicyclo[2.2.1]heptan-2-yl)cyclobut-3-ene-1,2-dione (Compound 16)

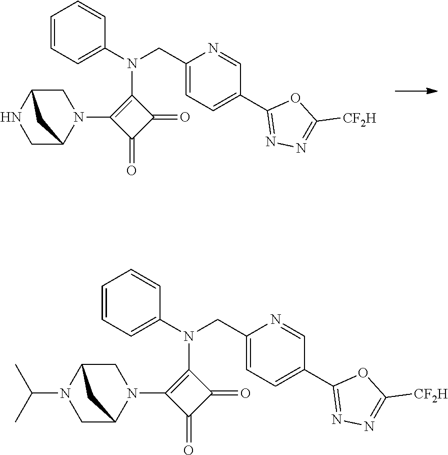 [SMILES: O=c1c(N(Cc2ccc(-c3nnc(C(F)F)o3)cn2)c2ccccc2)c(N2C[C@@H]3C[C@H]2CN3)c1=O ; CC(C)N1C[C@@H]2C[C@H]1CN2c1c(N(Cc2ccc(-c3nnc(C(F)F)o3)cn2)c2ccccc2)c(=O)c1=O]

3-((1S,4S)-2,5-diazabicyclo[2.2.1]heptan-2-yl)-4-(((5-(5-(difluoromethyl)-1,3,4-oxadiazol-2-yl)pyridin-2-yl)methyl)(phenyl)amino)cyclobut-3-ene-1,2-dione (0.090 g, 0.188 mmol) prepared in Step 2 of Example 15 and acetone (0.021 mL, 0.282 mmol) were dissolved in dichloromethane (4 mL) at room temperature. To the reaction solution, sodium triacetoxyborohydride (0.080 g, 0.376 mmol) was added and stirred for 18 hours at the same temperature. A saturated aqueous solution of sodium hydrogen carbonate was poured into the reaction mixture, followed by extraction with dichloromethane. The mixture was filtered through a plastic filter to remove the solid residue and the aqueous layer and then concentrated under reduced pressure. The concentrate was purified by column chromatography (SiO₂, 4 g cartridge; methanol/dichloromethane=0% to 5%) and concentrated to obtain the title compound (0.056 g, 57.2%) as an orange solid.

¹H NMR (400 MHz, CDCl₃) δ 9.25 (d, J=2.0 Hz, 1H), 8.38 (dd, J=8.2, 2.1 Hz, 1H), 7.65 (d, J=8.1 Hz, 1H), 7.37 (t, J=7.8 Hz, 2H), 7.24 (t, J=7.4 Hz, 1H), 7.19 (d, J=7.7 Hz, 2H), 7.08-6.82 (m, 1H), 5.62-5.52 (m, 2H), 3.49 (brs, 1H), 3.03 (s, 1H), 2.46 (s, 2H), 1.88-1.57 (m, 4H), 1.04-1.03 (m, 3H), 0.99-0.95 (m, 4H); LRMS (ES) m/z 521.8 (M⁺+1).

### Example 17: Synthesis of 3-(((5-(5-(difluoromethyl)-1,3,4-oxadiazol-2-yl)pyridin-2-yl)methyl)(phenyl)amino)-4-((1S,4S)-5-(oxetan-3-yl)-2,5-diazabicyclo[2.2.1]heptan-2-yl)cyclobut-3-ene-1,2-dione (Compound 17)

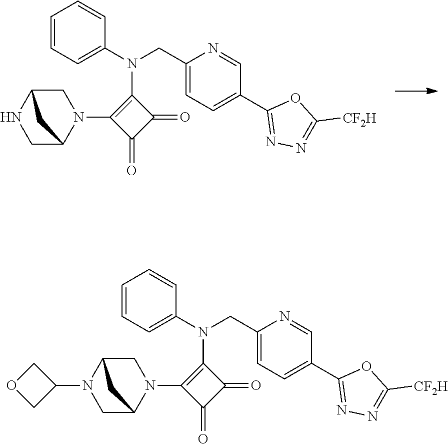 [SMILES: O=c1c(N(Cc2ccc(-c3nnc(C(F)F)o3)cn2)c2ccccc2)c(N2C[C@@H]3C[C@H]2CN3)c1=O ; O=c1c(N(Cc2ccc(-c3nnc(C(F)F)o3)cn2)c2ccccc2)c(N2C[C@@H]3C[C@H]2CN3C2COC2)c1=O]

3-((1S,4S)-2,5-diazabicyclo[2.2.1]heptan-2-yl)-4-(((5-(5-(difluoromethyl)-1,3,4-oxadiazol-2-yl)pyridin-2-yl)methyl)(phenyl)amino)cyclobut-3-ene-1,2-dione (0.100 g, 0.209 mmol) prepared in Step 2 of Example 15 and 3-oxetanone (0.018 mL, 0.314 mmol) were dissolved in dichloromethane (4 mL) at room temperature. To the reaction solution, sodium triacetoxyborohydride (0.089 g, 0.418 mmol) was added and stirred for 18 hours at the same temperature. A saturated aqueous solution of sodium hydrogen carbonate was poured into the reaction mixture, followed by extraction with dichloromethane. The mixture was filtered through a plastic filter to remove the solid residue and the aqueous layer and then concentrated under reduced pressure. The concentrate was purified by column chromatography (SiO₂, 4 g cartridge; methanol/dichloromethane=0% to 2.5%) and concentrated to obtain the title compound (0.036 g, 32.2%) as an orange solid.

¹H NMR (400 MHz, CDCl₃) δ 9.27 (s, 1H), 8.39 (dd, J=8.1, 1.8 Hz, 1H), 7.64 (d, J=8.2 Hz, 1H), 7.38 (t, J=7.7 Hz, 2H), 7.26-7.24 (m, 1H), 7.21 (d, J=8.0 Hz, 2H), 7.08-6.82 (m, 1H), 5.62-5.51 (m, 2H), 4.65-4.59 (m, 2H), 4.44 (brs, 2H), 3.77 (brs, 2H), 3.20 (brs, 2H), 2.80-2.72 (m, 2H), 1.82-1.52 (m, 4H); LRMS (ES) m/z 535.8 (M⁺+1).

### Example 18: Synthesis of 3-(((5-(5-(difluoromethyl)-1,3,4-oxadiazol-2-yl)pyridin-2-yl)methyl)(3-(pyridin-3-yl)phenyl)amino)-4-(4-methylpiperazin-1-yl)cyclobut-3-ene-1,2-dione (Compound 18)

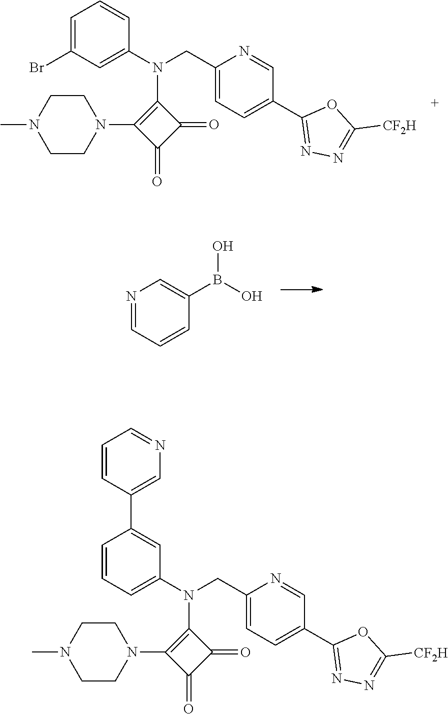 [SMILES: CN1CCN(c2c(N(Cc3ccc(-c4nnc(C(F)F)o4)cn3)c3cccc(Br)c3)c(=O)c2=O)CC1 ; OB(O)c1cccnc1 ; CN1CCN(c2c(N(Cc3ccc(-c4nnc(C(F)F)o4)cn3)c3cccc(-c4cccnc4)c3)c(=O)c2=O)CC1]

3-((3-Bromophenyl)((5-(5-(difluoromethyl)-1,3,4-oxadiazol-2-yl)pyridin-2-yl)methyl)amino)-4-(4-methylpiperazin-1-yl)cyclobut-3-ene-1,2-dione (0.250 g, 0.447 mmol), pyridin-3-ylboronic acid (0.071 g, 0.581 mmol), [1,1′-bis(di-tert-butylphosphino)ferrocene]palladium(II) dichloride (Pd(dtbpf)Cl₂, 0.029 g, 0.045 mmol), and cesium carbonate (0.364 g, 1.117 mmol) were mixed with 1,4-dioxane (9 mL)/water (3 mL) and heated at 100° C. for 20 hours by microwave irradiation. Then, the reaction was completed by lowering the reaction temperature to room temperature. Water was poured into the reaction mixture, and the mixture was extracted with ethyl acetate. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; methanol/dichloromethane=0% to 10%) and concentrated to obtain the title compound (0.100 g, 40.1%) as a brown oil.

¹H NMR (400 MHz, CDCl₃) δ 8.78 (d, J=1.6 Hz, 1H), 8.64 (d, J=3.8 Hz, 1H), 7.88-7.76 (m, 3H), 7.66 (t, J=7.6 Hz, 1H), 7.47 (t, J=7.8 Hz, 1H), 7.42-7.39 (m, 2H), 7.18 (s, 1H), 7.07 (dd, J=7.9, 1.3 Hz, 1H), 7.04 (s, 0.25H), 6.91 (s, 0.5H), 6.78 (s, 0.25H), 5.61 (s, 2H), 3.32 (br s, 4H), 2.23 (br s, 4H), 2.16 (s, 3H); LRMS (ES) m/z 558.4 (M⁺+1).

### Example 19: Synthesis of 3-(((5-(5-(difluoromethyl)-1,3,4-oxadiazol-2-yl)pyridin-2-yl)methyl)(3-(pyridin-4-y 1)phenyl)amino)-4-(4-methylpiperazin-1-yl)cyclobut-3-ene-1,2-dione (Compound 19)

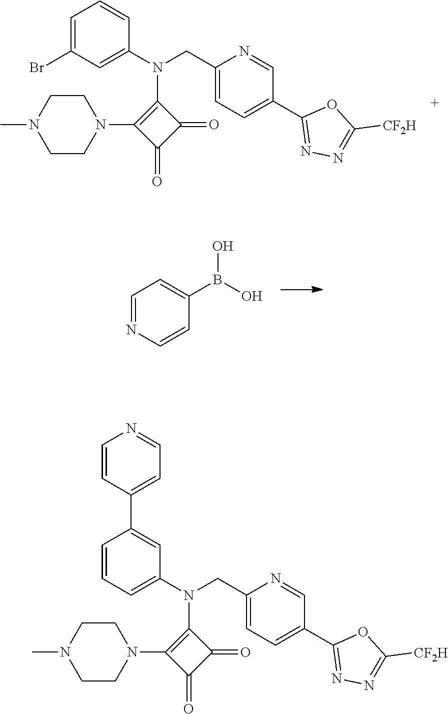 [SMILES: CN1CCN(c2c(N(Cc3ccc(-c4nnc(C(F)F)o4)cn3)c3cccc(Br)c3)c(=O)c2=O)CC1 ; OB(O)c1ccncc1 ; CN1CCN(c2c(N(Cc3ccc(-c4nnc(C(F)F)o4)cn3)c3cccc(-c4ccncc4)c3)c(=O)c2=O)CC1]

3-((3-Bromophenyl)((5-(5-(difluoromethyl)-1,3,4-oxadiazol-2-yl)pyridin-2-yl)methyl)amino)-4-(4-methylpiperazin-1-yl)cyclobut-3-ene-1,2-dione (0.200 g, 0.358 mmol), pyridin-4-ylboronic acid (0.057 g, 0.465 mmol), [1,1′-bis(di-tert-butylphosphino)ferrocene]palladium(II) dichloride (Pd(dtbpf)Cl₂, 0.023 g, 0.036 mmol), and cesium carbonate (0.291 g, 0.894 mmol) were mixed with 1,4-dioxane (9 mL)/water (3 mL) and heated at 100° C. for 15 minutes by microwave irradiation. Then, the reaction was completed by lowering the reaction temperature to room temperature. Water was poured into the reaction mixture, and the mixture was extracted with ethyl acetate. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; methanol/dichloromethane=0% to 10%) and concentrated to obtain the title compound (0.060 g, 30.1%) as a black oil.

¹H NMR (400 MHz, CDCl₃) (8.71 (d, J=4.4 Hz, 2H), 7.89 (d, J=8.1 Hz, 1H), 7.80 (dd, J=10.1, 1.1 Hz, 1H), 7.67 (t, J=7.6 Hz, 1H), 7.50-7.43 (m, 4H), 7.23 (s, 1H), 7.11-7.09 (m, 1H), 7.04 (s, 0.25), 6.91 (s, 0.5H), 6.78 (s, 0.25H), 5.62 (s, 2H), 3.31 (br s, 4H), 2.22 (br s, 4H), 3.16 (s, 3H); LRMS (ES) m/z 558.4 (M⁺+1).

### Example 20: Synthesis of 3-(((5-(5-(difluoromethyl)-1,3,4-oxadiazol-2-yl)-3-fluoropyridin-2-yl)methyl)(phenyl)amino)-4-((1S,4S)-5-methyl-2,5-diazabicyclo[2.2.1]heptan-2-yl)cyclobut-3-ene-1,2-dione (Compound 20)

### [Step 1] Synthesis of tert-butyl (1S,4S)-5-(2-(((5-(5-(difluoromethyl)-1.3.4-oxadiazol-2-yl)-3-fluoropyridin-2-yl]methyl)(phenyl)amino)-3,4-dioxocyclobut-1-en-1-yl)-2.5-diazabicyclo[2.2.1]heptane-2-carboxylate

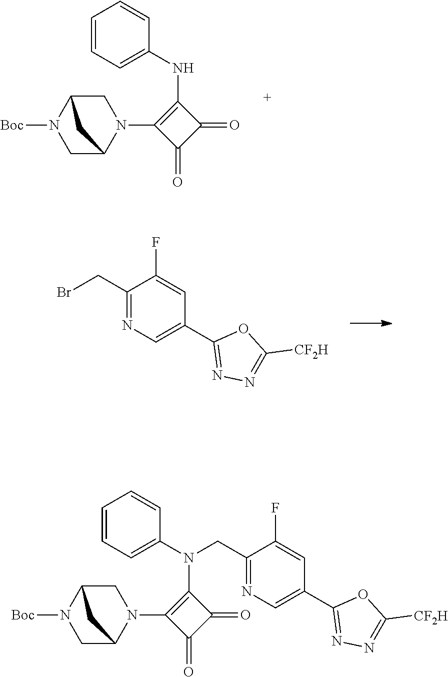 [SMILES: CC(C)(C)OC(=O)N1C[C@@H]2C[C@H]1CN2c1c(Nc2ccccc2)c(=O)c1=O ; Fc1cc(-c2nnc(C(F)F)o2)cnc1CBr ; CC(C)(C)OC(=O)N1C[C@@H]2C[C@H]1CN2c1c(N(Cc2ncc(-c3nnc(C(F)F)o3)cc2F)c2ccccc2)c(=O)c1=O]

Tert-butyl (1S,4S)-5-(3,4-dioxo-2-(phenylamino)cyclobut-1-en-1-yl)-2,5-diazabicyclo[2.2.1]heptane-2-carboxylate (0.500 g, 1.353 mmol) prepared in Step 1 of Example 13, 2-(bromomethyl)-3-fluoro-5-(5-(difluoromethyl)-1,3,4-oxadiazol-2-yl)pyridine (0.459 g, 1.489 mmol), potassium carbonate (0.281 g, 2.030 mmol), and potassium iodide (0.022 g, 0.135 mmol) were dissolved in N,N-dimethylformamide (4 mL) at room temperature, and the reaction solution was stirred for 18 hours at 60° C. Then, the reaction was completed by lowering the reaction temperature to room temperature. The solvent was removed from the reaction mixture under reduced pressure to obtain a concentrate. Into the concentrate, a saturated aqueous solution of sodium hydrogen carbonate was poured, and the reaction mixture was extracted with dichloromethane. The mixture was filtered through a plastic filter to remove the solid residue and the aqueous layer and then concentrated under reduced pressure. The concentrate was purified by column chromatography (SiO₂, 12 g cartridge; ethyl acetate/hexane=30% to 70%) and concentrated to obtain the title compound (0.285 g, 35.3%) as a brown solid.

### [Step 2] Synthesis of 3-((1S,4S)-2.5-diazabicyclo[2.2.1]heptan-2-yl)-4-(((5-(5-(difluoromethyl)-1.3.4-oxadiazol-2-yl)-3-fluoropyridin-2-yl)methyl)(phenyl)amino)cyclobut-3-ene-1.2-dione

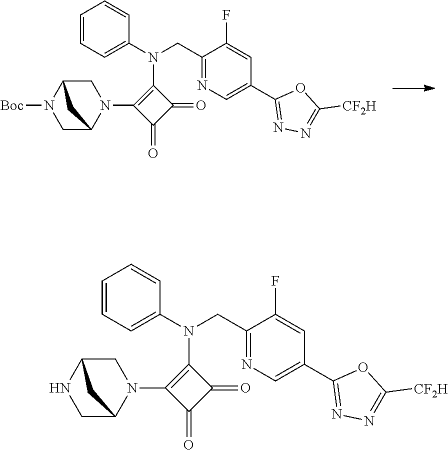 [SMILES: CC(C)(C)OC(=O)N1C[C@@H]2C[C@H]1CN2c1c(N(Cc2ncc(-c3nnc(C(F)F)o3)cc2F)c2ccccc2)c(=O)c1=O ; O=c1c(N(Cc2ncc(-c3nnc(C(F)F)o3)cc2F)c2ccccc2)c(N2C[C@@H]3C[C@H]2CN3)c1=O]

Tert-butyl (1S,4S)-5-(2-(((5-(5-(difluoromethyl)-1,3,4-oxadiazol-2-yl)-3-fluoropyridin-2-yl)meth yl)(phenyl)amino)-3,4-dioxocyclobut-1-en-1-yl)-2,5-diazabicyclo[2.2.1]heptane-2-carboxylate (0.285 g, 0.478 mmol) prepared in Step 1 and trifluoroacetic acid (0.256 mL, 3.344 mmol) were dissolved in dichloromethane (4 mL) at room temperature, and the reaction solution was stirred for 18 hours at the same temperature. A saturated aqueous solution of sodium hydrogen carbonate was poured into the reaction mixture, followed by extraction with dichloromethane. The mixture was filtered through a plastic filter to remove the solid residue and the aqueous layer and then concentrated under reduced pressure. The concentrate was purified by column chromatography (SiO₂, 4 g cartridge; ethyl acetate/hexane=0% to 30%) and concentrated to obtain the title compound (0.206 g, 86.9%) as a pale brown solid.

### [Step 3] Synthesis of Compound 20

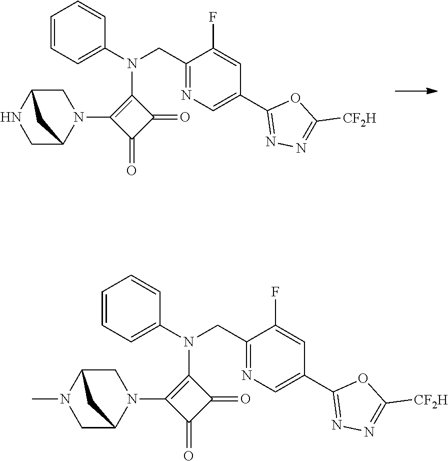 [SMILES: O=c1c(N(Cc2ncc(-c3nnc(C(F)F)o3)cc2F)c2ccccc2)c(N2C[C@@H]3C[C@H]2CN3)c1=O ; CN1C[C@@H]2C[C@H]1CN2c1c(N(Cc2ncc(-c3nnc(C(F)F)o3)cc2F)c2ccccc2)c(=O)c1=O]

3-((1S,4S)-2,5-diazabicyclo[2.2.1]heptan-2-yl)-4-(((5-(5-(difluoromethyl)-1,3,4-oxadiazol-2-yl)-3-fluoropyridin-2-yl)methyl)(phenyl)amino)cyclobut-3-ene-1,2-dione (0.100 g, 0.201 mmol) prepared in Step 2 and formaldehyde (0.009 g, 0.302 mmol) were dissolved in dichloromethane (4 mL) at room temperature. To the reaction solution, sodium triacetoxyborohydride (0.085 g, 0.403 mmol) was added and stirred for 18 hours at the same temperature. A saturated aqueous solution of sodium hydrogen carbonate was poured into the reaction mixture, followed by extraction with dichloromethane. The mixture was filtered through a plastic filter to remove the solid residue and the aqueous layer and then concentrated under reduced pressure. The concentrate was purified by column chromatography (SiO₂, 4 g cartridge; methanol/dichloromethane=0% to 5%) and concentrated to obtain the title compound (0.067 g, 65.2%) as a yellow solid.

¹H NMR (400 MHz, CDCl₃) δ 9.05 (s, 1H), 8.06 (d, J=9.3 Hz, 1H), 7.34 (t, J=7.1 Hz, 2H), 7.22 (t, J=7.4 Hz, 1H), 7.14 (d, J=8.0 Hz, 2H), 7.08-6.82 (m, 1H), 5.71-5.66 (m, 2H), 3.18 (brs, 1H), 2.77 (brs, 3H), 2.61-2.59 (m, 1H), 2.30 (brs, 3H), 1.86 (d, J=9.8 Hz, 1H), 1.57 (d, J=10.1 Hz, 1H); LRMS (ES) m/z 511.8 (M⁺+1).

### Example 21: Synthesis of 3-(((5-(5-(difluoromethyl)-1,3,4-oxadiazol-2-yl)-3-fluoropyridin-2-yl)methyl)(phenyl)amino)-4-((1S,4S)-5-(oxetan-3-yl)-2,5-diazabicyclo[2.2.1]heptan-2-yl)cyclobut-3-ene-1,2-dione (Compound 21)

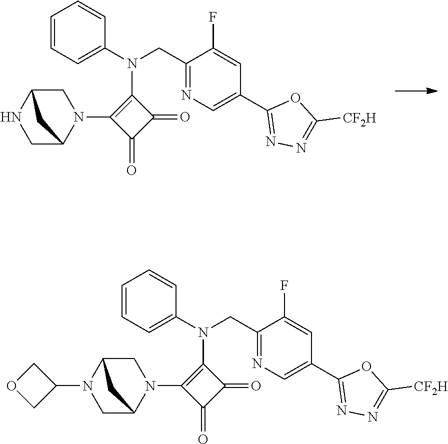 [SMILES: O=c1c(N(Cc2ncc(-c3nnc(C(F)F)o3)cc2F)c2ccccc2)c(N2C[C@@H]3C[C@H]2CN3)c1=O ; O=c1c(N(Cc2ncc(-c3nnc(C(F)F)o3)cc2F)c2ccccc2)c(N2C[C@@H]3C[C@H]2CN3C2COC2)c1=O]

3-((1S,4S)-2,5-diazabicyclo[2.2.1]heptan-2-yl)-4-(((5-(5-(difluoromethyl)-1,3,4-oxadiazol-2-yl)-3-fluoropyridin-2-yl)methyl)(phenyl)amino)cyclobut-3-ene-1,2-dione (0.100 g, 0.201 mmol) prepared in Step 2 of Example 20 and 3-oxetanone (0.019 mL, 0.302 mmol) were dissolved in dichloromethane (4 mL) at room temperature. To the reaction solution, sodium triacetoxyborohydride (0.085 g, 0.403 mmol) was added and stirred for 18 hours at the same temperature. A saturated aqueous solution of sodium hydrogen carbonate was poured into the reaction mixture, followed by extraction with dichloromethane. The mixture was filtered through a plastic filter to remove the solid residue and the aqueous layer and then concentrated under reduced pressure. The concentrate was purified by column chromatography (SiO₂, 4 g cartridge; methanol/dichloromethane=0% to 2.5%) and concentrated to obtain the title compound (0.019 g, 17.1%) as a yellow solid.

¹H NMR (400 MHz, CDCl₃) δ 9.09 (s, 1H), 8.10 (dd, J=29.3, 20.0 Hz, 1H), 7.37 (t, J=7.7 Hz, 2H), 7.26-7.24 (m, 1H), 7.18 (d, J=7.6 Hz, 2H), 7.08-6.82 (m, 1H), 5.68 (s, 2H), 4.68-4.63 (m, 2H), 4.50-4.45 (m, 2H), 3.81-3.80 (m, 1H), 3.22 (brs, 1H), 2.82 (brs, 1H), 2.75 (brs, 1H), 1.83-1.59 (m, 4H), 1.31-1.30 (m, 1H); LRMS (ES) m/z 553.7 (M⁺+1).

### Example 22: Synthesis of 3-((4-(5-(difluoromethyl)-1,3,4-oxadiazol-2-yl)-2-fluorobenzyl)(3,4-difluorophenyl)amino)-4-(4-methylpiperazin-1-yl)cyclobut-3-ene-1,2-dione (Compound 22)

### [Step 1] Synthesis of 3-((3,4-difluorophenyl)amino)-4-methoxycyclobut-3-ene-1,2-dione

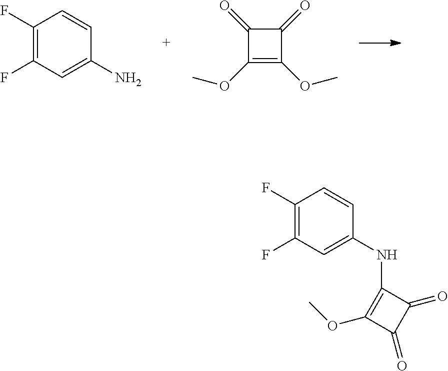

3,4-Difluoroaniline (1.000 g, 7.745 mmol) and 3,4-dimethoxycyclobut-3-ene-1,2-dione (1.101 g, 7.745 mmol) were dissolved in methanol (30 mL) at room temperature and stirred for 18 hours at the same temperature. The precipitated solid was filtered, washed with methanol and dried to obtain the title compound (1.610 g, 86.9%) as a pale yellow solid.

### [Step 2] Synthesis of tert-butyl 4-(2-((3,4-difluorophenyl)amino)-3,4-dioxocyclobut-1-en-1-yl)piperazine-1-carboxylate

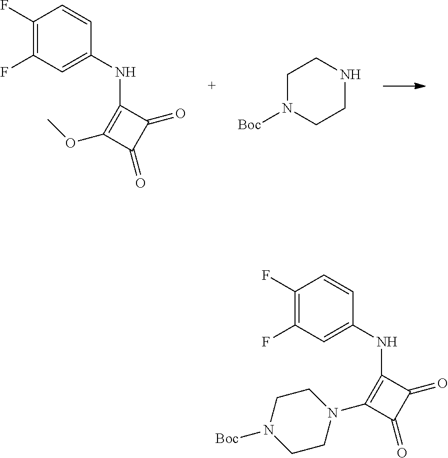 [SMILES: COc1c(Nc2ccc(F)c(F)c2)c(=O)c1=O ; CC(C)(C)OC(=O)N1CCNCC1 ; CC(C)(C)OC(=O)N1CCN(c2c(Nc3ccc(F)c(F)c3)c(=O)c2=O)CC1]

3-((3,4-Difluorophenyl)amino)-4-methoxycyclobut-3-ene-1,2-dione (1.500 g, 6.271 mmol) prepared in Step 2, tert-butyl piperazine-1-carboxylate (1.168 g, 6.271 mmol), and N,N-diisopropylethylamine (1.092 mL, 6.271 mmol) were dissolved in methanol (30 mL) at room temperature, and the reaction solution was stirred for 18 hours at the same temperature. Methanol was added to the reaction mixture and stirred to precipitate a solid, and the precipitated solid was filtered, washed with methanol, and dried to obtain the title compound (1.770 g, 71.7%) as a pale green solid.

### [Step 3] Synthesis of tert-butyl 4-(2-((4-(5-(difluoromethyl)-1,3,4-oxadiazol-2-yl)-2-fluorobenzyl)(3,4-difluorophenyl)amino)-3,4-dioxocyclobut-1-en-1-yl)piperazine-1-carboxylate

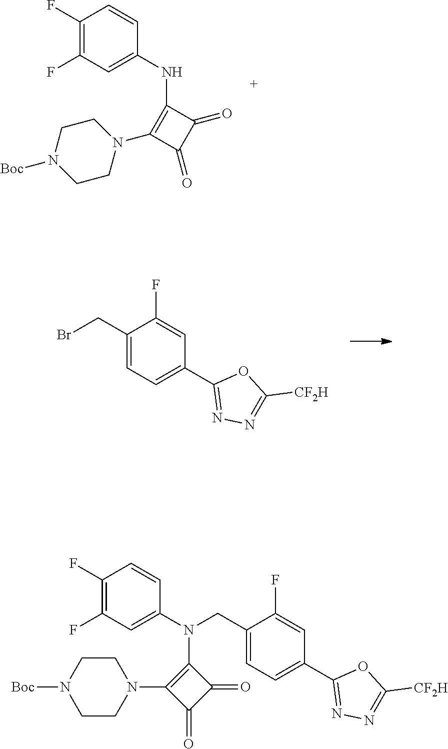 [SMILES: CC(C)(C)OC(=O)N1CCN(c2c(Nc3ccc(F)c(F)c3)c(=O)c2=O)CC1 ; Fc1cc(-c2nnc(C(F)F)o2)ccc1CBr ; CC(C)(C)OC(=O)N1CCN(c2c(N(Cc3ccc(-c4nnc(C(F)F)o4)cc3F)c3ccc(F)c(F)c3)c(=O)c2=O)CC1]

Tert-butyl 4-(2-((3,4-difluorophenyl)amino)-3,4-dioxocyclobut-1-en-1-yl)piperazine-1-carboxylate (0.500 g, 1.271 mmol) prepared in Step 2 and sodium hydride (60.00%, 0.056 g, 1.398 mmol) were dissolved in N,N-dimethylformamide (20 mL) at 0° C. To the reaction solution, 2-(4-(bromomethyl)-3-fluorophenyl)-5-(difluoromethyl)-1,3,4-oxadiazole (0.429 g, 1.398 mmol) was added and stirred for 18 hour at room temperature. After removing the solvent from the reaction mixture under reduced pressure, the concentrate was purified by column chromatography (SiO₂, 12 g cartridge; ethyl acetate/hexane=5% to 40%) and concentrated to obtain the title compound (0.516 g, 65.5%) as a pale yellow solid.

### [Step 4] Synthesis of 3-((4-(5-(difluoromethyl)-1,3,4-oxadiazol-2-yl)-2-fluorobenzyl)(3,4-difluorophenyl)amino)-4-(piperazin-1-yl)cyclobut-3-ene-1,2-dione

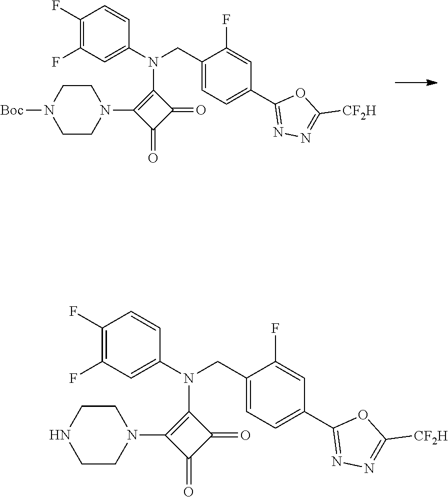 [SMILES: CC(C)(C)OC(=O)N1CCN(c2c(N(Cc3ccc(-c4nnc(C(F)F)o4)cc3F)c3ccc(F)c(F)c3)c(=O)c2=O)CC1 ; O=c1c(N2CCNCC2)c(N(Cc2ccc(-c3nnc(C(F)F)o3)cc2F)c2ccc(F)c(F)c2)c1=O]

Tert-butyl 4-(2-((4-(5-(difluoromethyl)-1,3,4-oxadiazol-2-yl)-2-fluorobenzyl)(3,4-difluorophenyl) amino)-3,4-dioxocyclobut-1-en-1-yl)piperazine-1-carboxylate (0.516 g, 0.833 mmol) prepared in Step 3 and trifluoroacetic acid (0.510 mL, 6.663 mmol) were dissolved in dichloromethane (5 mL) at room temperature, and the reaction solution was stirred for 18 hours at the same temperature. A saturated aqueous solution of sodium hydrogen carbonate and dichloromethane were added to the reaction mixture and stirred to precipitate a solid, and the precipitated solid was filtered. The filtrate was washed with dichloromethane and dried to obtain the title compound (0.361 g, 83.4%) as a yellow solid.

### [Step 5] Synthesis of Compound 22

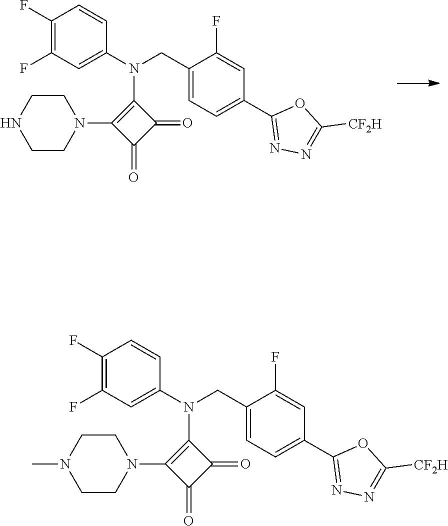 [SMILES: O=c1c(N2CCNCC2)c(N(Cc2ccc(-c3nnc(C(F)F)o3)cc2F)c2ccc(F)c(F)c2)c1=O ; CN1CCN(c2c(N(Cc3ccc(-c4nnc(C(F)F)o4)cc3F)c3ccc(F)c(F)c3)c(=O)c2=O)CC1]

3-((4-(5-(Difluoromethyl)-1,3,4-oxadiazol-2-yl)-2-fluorobenzyl)(3,4-difluorophenyl) amino)-4-(piperazin-1-yl)cyclobut-3-ene-1,2-dione (0.100 g, 0.193 mmol) prepared in Step 4 and formaldehyde (38.00% solution, 0.021 mL, 0.289 mmol) were dissolved in dichloromethane (4 mL) at room temperature. To the reaction solution, sodium triacetoxyborohydride (0.082 g, 0.385 mmol) was added and stirred for 18 hours at the same temperature. A saturated aqueous solution of sodium hydrogen carbonate was poured into the reaction mixture, followed by extraction with dichloromethane. The mixture was filtered through a plastic filter to remove the solid residue and the aqueous layer and then concentrated under reduced pressure. The concentrate was purified by column chromatography (SiO₂, 4 g cartridge; methanol/dichloromethane=0% to 5%) and concentrated to obtain the title compound (0.044 g, 42.8%) as a white solid.

¹H NMR (400 MHz, CDCl₃) δ 7.90 (d, J=8.0 Hz, 1H), 7.81 (d, J=10.2 Hz, 1H), 7.63 (t, J=7.7 Hz, 1H), 7.17 (q, J=9.0 Hz, 1H), 7.06-6.85 (m, 2H), 6.77-6.75 (m, 1H), 5.52 (s, 1H), 3.37 (s, 4H), 2.35 (s, 4H), 2.28 (s, 3H); LRMS (ES) m/z 534.7 (M⁺+1)

### Example 23: Synthesis of 3-((4-(5-(difluoromethyl)-1,3,4-oxadiazol-2-yl)-2-fluorobenzyl)(3,4-difluorophenyl)amino)-4-(4-(oxetan-3-yl)piperazin-1-yl)cyclobut-3-ene-1,2-dione (Compound 23)

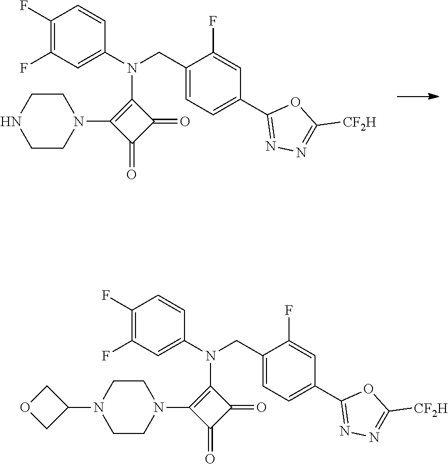 [SMILES: O=c1c(N2CCNCC2)c(N(Cc2ccc(-c3nnc(C(F)F)o3)cc2F)c2ccc(F)c(F)c2)c1=O ; O=c1c(N2CCN(C3COC3)CC2)c(N(Cc2ccc(-c3nnc(C(F)F)o3)cc2F)c2ccc(F)c(F)c2)c1=O]

3-((4-(5-(Difluoromethyl)-1,3,4-oxadiazol-2-yl)-2-fluorobenzyl)(3,4-difluorophenyl) amino)-4-(piperazin-1-yl)cyclobut-3-ene-1,2-dione (0.065 g, 0.125 mmol) prepared in Step 4 of Example 23 and oxetan-3-one (0.012 mL, 0.188 mmol) were dissolved in dichloromethane (4 mL) at room temperature. To the reaction solution, sodium triacetoxyborohydride (0.053 g, 0.250 mmol) was added and stirred for 18 hours at the same temperature. A saturated aqueous solution of sodium hydrogen carbonate was poured into the reaction mixture, followed by extraction with dichloromethane. The mixture was filtered through a plastic filter to remove the solid residue and the aqueous layer and then concentrated under reduced pressure. The concentrate was purified by column chromatography (SiO₂, 4 g cartridge; methanol/dichloromethane=0% to 2.5%) and concentrated to obtain the title compound (0.016 g, 22.2%) as a white solid.

¹H NMR (400 MHz, CDCl₃) δ 7.90 (dd, J=8.0, 1.2 Hz, 1H), 7.80 (dd, J=10.1, 1.2 Hz, 1H), 7.62 (t, J=7.7 Hz, 1H), 7.17 (q, J=9.0 Hz, 1H), 7.06-6.85 (m, 2H), 6.77-6.75 (m, 1H), 5.52 (s, 2H), 4.64 (t, J=6.5 Hz, 2H), 4.52 (t, J=6.1 Hz, 2H), 3.50-3.39 (m, 5H), 2.25 (brs, 4H); LRMS (ES) m/z 576.3 (M⁺+1).

### Example 24: Synthesis of 3-morpholino-4-[N-[[4-[5-(trifluoromethyl)-1,3,4-oxadiazol-2-yl]phenyl]methyl]anilino]cyclobut-3-ene-1,2-dione (Compound 24)

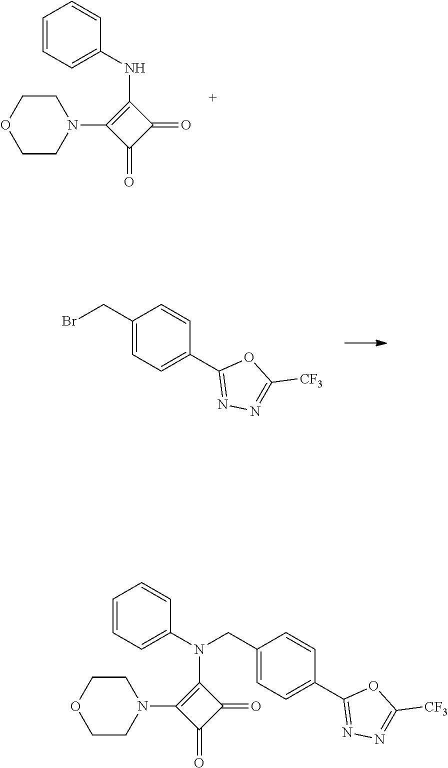 [SMILES: O=c1c(Nc2ccccc2)c(N2CCOCC2)c1=O ; FC(F)(F)c1nnc(-c2ccc(CBr)cc2)o1 ; O=c1c(N2CCOCC2)c(N(Cc2ccc(-c3nnc(C(F)(F)F)o3)cc2)c2ccccc2)c1=O]

3-Morpholino-4-(phenylamino)cyclobut-3-ene-1,2-dione (0.500 g, 1.936 mmol) prepared in Step 1 of Example 2 and sodium hydride (60.00%, 0.085 g, 2.125 mmol) were dissolved in N,N-dimethylformamide (10 mL) at 0° C. To the reaction solution, 2-[4-(bromomethyl)phenyl)-5-(trifluoromethyl)-1,3,4-oxadiazole (0.624 g, 2.03 mmol) was added and stirred for 18 hours at room temperature. The solvent was removed from the reaction mixture under reduced pressure to obtain a concentrate. Into the concentrate, a saturated aqueous solution of sodium hydrogen carbonate was poured, and the reaction mixture was extracted with dichloromethane. The mixture was filtered through a plastic filter to remove the solid residue and the aqueous layer and then concentrated under reduced pressure. The concentrate was purified by column chromatography (SiO₂, 12 g cartridge; ethyl acetate/hexane=10% to 60%) and concentrated. Dichloromethane and diethyl ether were added to the obtained product and stirred to precipitate a solid, and the precipitated solid was filtered. The filtrate was washed with diethyl ether and dried to obtain the title compound (0.196 g, 20.90%, white solid).

¹H NMR (400 MHz, DMSO-d₆) δ 8.06 (d, J=8.4 Hz, 2H), 7.60 (d, J=8.4 Hz, 2H), 7.41 (t, J=7.8 Hz, 2H), 7.26-7.21 (m, 3H), 5.57 (s, 2H), 3.51 (t, J=4.8 Hz, 4H), 3.27 (brs, 4H); LRMS (ES) m/z 485.7 (M⁺+1).

### Activity Measurement and Analysis Protocol of Compounds According to Present Invention

### <Experimental Example 1> Confirmation of Inhibition of HDAC Enzyme Activity (In Vitro)

A selective HDAC6 inhibitor is important for the selectivity of HDAC1 inhibition as a cause of side effects. In order to confirm the importance, HDAC1/6 enzyme selectivity and cell selectivity (HDAC1: Histone acetylation/HDAC6: Tubulin acetylation) were confirmed.

### 1. Experimental Method

The HDAC enzyme inhibition ability of the test substance was measured using the HDAC1 Fluorimetric Drug Discovery Assay Kit (Enzo life sciences: BML-AK511) and the HDAC6 human recombinant (Calbiochem: 382180). In the HDAC1 assay, the test substance was treated at concentrations of 100, 1000, and 10000 nM, and in the HDAC6 assay, the test substance was treated at concentrations of 0.1, 1, 10, 100, and 1000 nM. After sample treatment, the samples were reacted at 37° C. for 60 minutes, followed by reaction at 37° C. for 30 minutes by treatment with a developer, and fluorescence intensities (Ex 390, Em 460) thereof were measured using FlexStatin3 (Molecular device).

### 2. Experimental Result

Table 2 shows the HDAC enzyme activity inhibition assay results obtained according to the above experimental method.

TABLE 2

| Compound | HDAC6 IC₅₀ (uM) | HDAC1 IC₅₀ (uM) |
|---|---|---|
| 1 | 0.022 | >10 |
| 2 | 0.026 | >10 |
| 3 | 0.027 | >10 |
| 4 | 0.031 | >10 |
| 5 | 0.029 | >10 |
| 6 | 0.032 | >10 |
| 7 | 0.036 | >10 |
| 8 | 0.035 | >10 |
| 9 | 0.054 | >10 |
| 10 | 0.056 | >10 |
| 11 | 0.060 | >10 |
| 12 | 0.030 | >10 |
| 13 | 0.024 | >10 |
| 14 | 0.024 | >10 |
| 15 | 0.022 | >10 |
| 16 | 0.028 | >10 |
| 17 | 0.043 | >10 |
| 18 | 0.051 | >10 |
| 19 | 0.102 | >10 |
| 20 | 0.039 | >10 |
| 21 | 0.054 | >10 |
| 22 | 0.064 | >10 |
| 23 | 0.050 | >10 |
| 24 | 0.161 | >10 |

1. A 1,3,4-oxadiazole derivative compound represented by the following Chemical Formula I, a stereoisomer thereof or a pharmaceutically acceptable salt thereof: 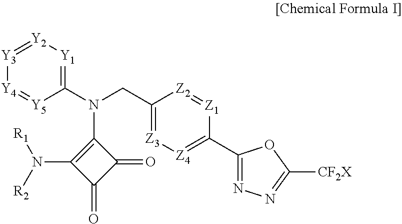
wherein:
R₁ and R₂ are —(C₁-C₄ alkyl), or R₁ and R₂ together with an adjacent nitrogen atom are linked to form a heterocycloalkyl, wherein at least one H of the heterocycloalkyl is substituted with —(C₁-C₄ alkyl), —(C₁-C₄ aminoalkyl), —(C₁-C₄ hydroxyalkyl), —(C₁-C₄ haloalkyl), -halo, or heterocycloalkyl;
X is —H or —F;
Y₁ to Y₅ are each independently N or CR₃, provided that Y₁ to Y₅ are not 3 or more N at the same time;
R₃ is —H, —(C₁-C₄ alkyl), —(C₁-C₄ aminoalkyl), —(C₁-C₄ hydroxyalkyl), —(C₁-C₄ haloalkyl), -halo, aryl or heteroaryl, wherein at least one —H of the aryl or heteroaryl is substituted with —(C₁-C₄ alkyl), —(C₁-C₄ aminoalkyl), —(C₁-C₄ hydroxyalkyl), —(C₁-C₄ haloalkyl), —OH, —O(C₁-C₄ alkyl), or -halo;
Z₁ to Z₄ are each independently N or CR₄, provided that Z₁ to Z₄ are not 3 or more N at the same time; and
R₄ is —H, —(C₁-C₄ alkyl), —(C₁-C₄ aminoalkyl), —(C₁-C₄ hydroxyalkyl), —(C₁-C₄ haloalkyl), -halo, aryl or heteroaryl, wherein at least one —H of the aryl or heteroaryl is each independently substituted with —(C₁-C₄ alkyl), —(C₁-C₄ aminoalkyl), —(C₁-C₄ hydroxyalkyl), —(C₁-C₄ haloalkyl), —OH, —O(C₁-C₄ alkyl), or -halo.

2. The 1,3,4-oxadiazole derivative compound, the stereoisomer thereof or the pharmaceutically acceptable salt thereof according to claim 1, wherein:
R₁ and R₂ together with an adjacent nitrogen atom are linked to form a heterocycloalkyl, wherein at least one H of the heterocycloalkyl is substituted with —(C₁-C₄ alkyl) or heterocycloalkyl;
X is —H or —F;
Y₁ to Y₅ are each independently CR₃;
R₃ is —H, -halo or heteroaryl, wherein at least one —H of the heteroaryl is substituted with —(C₁-C₄ alkyl), —(C₁-C₄ aminoalkyl), —(C₁-C₄ hydroxyalkyl), —(C₁-C₄ haloalkyl), —OH, —O(C₁-C₄ alkyl), or -halo;
Z₁ to Z₄ are each independently N or CR₄, provided that Z₁ to Z₄ are not 3 or more N at the same time; and
R₄ is —H or -halo.

3. The 1,3,4-oxadiazole derivative compound, the stereoisomer thereof or the pharmaceutically acceptable salt thereof according to claim 1, wherein:
R₁ and R₂ together with an adjacent nitrogen atom are linked to form a 4-12 membered heterocycloalkyl, wherein at least one H of the 4-12 membered heterocycloalkyl ring is substituted with —(C₁-C₄ alkyl), —(C₁-C₄ aminoalkyl), —(C₁-C₄ hydroxyalkyl), —(C₁-C₄ haloalkyl), -halo, or 3-6 membered heterocycloalkyl.

4. The 1,3,4-oxadiazole derivative compound, the stereoisomer thereof or the pharmaceutically acceptable salt thereof according to claim 3, wherein:
R₁ and R₂ together with an adjacent nitrogen atom are linked to form 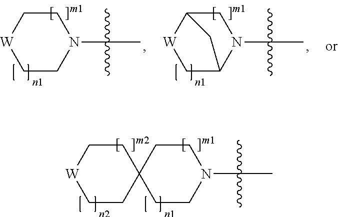
wherein at least one H of the 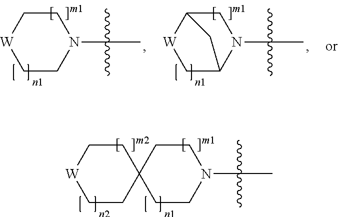
is substituted with —(C₁-C₄ alkyl), —(C₁-C₄ aminoalkyl), —(C₁-C₄ hydroxyalkyl), —(C₁-C₄ haloalkyl), or -halo;
W is NR₅, O, S, or S(═O)₂;
R₅ is —(C₁-C₄ alkyl) or 3-6 membered heterocycloalkyl; and
n1, n2, ml, and m2 are each independently 0, 1, or 2.

5. The 1,3,4-oxadiazole derivative compound, the stereoisomer thereof or pharmaceutically acceptable salt thereof according to claim 4, wherein;
R₁ and R₂ together with an adjacent nitrogen atom are linked to form 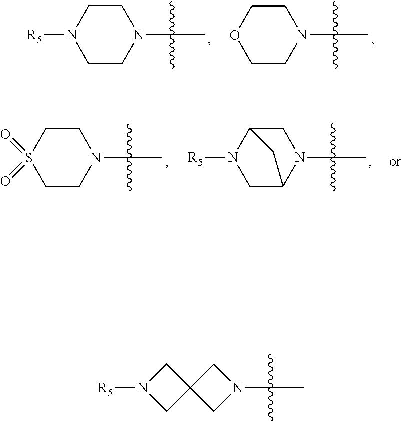
wherein at least one H of the 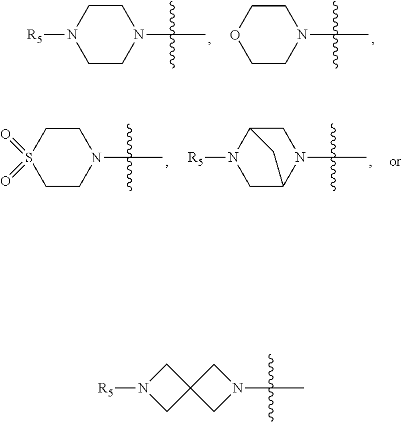
is substituted with —(C₁-C₄ alkyl), —(C₁-C₄ aminoalkyl), —(C₁-C₄ hydroxyalkyl), —(C₁-C₄ haloalkyl), or -halo; and
—R₅ is —(C₁-C₄ alkyl) or

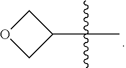 [SMILES: *C1COC1]

6. The 1,3,4-oxadiazole derivative compound, the stereoisomer thereof or the pharmaceutically acceptable salt thereof according to claim 1, wherein the 1,3,4-oxadiazole derivative compound is one of following compounds:

| Compound | |
|---|---|
| No. | Structure |
| 1 | 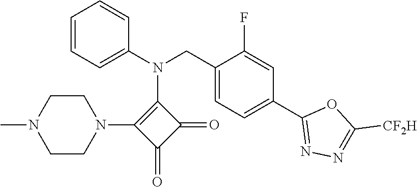 [SMILES: CN1CCN(c2c(N(Cc3ccc(-c4nnc(C(F)F)o4)cc3F)c3ccccc3)c(=O)c2=O)CC1] |
| 2 | 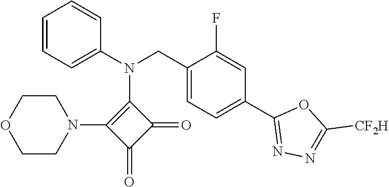 [SMILES: O=c1c(N2CCOCC2)c(N(Cc2ccc(-c3nnc(C(F)F)o3)cc2F)c2ccccc2)c1=O] |
| 3 | 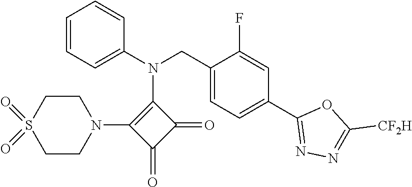 [SMILES: O=c1c(N2CCS(=O)(=O)CC2)c(N(Cc2ccc(-c3nnc(C(F)F)o3)cc2F)c2ccccc2)c1=O] |
| 4 | 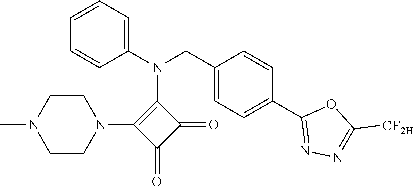 [SMILES: CN1CCN(c2c(N(Cc3ccc(-c4nnc(C(F)F)o4)cc3)c3ccccc3)c(=O)c2=O)CC1] |
| 5 | 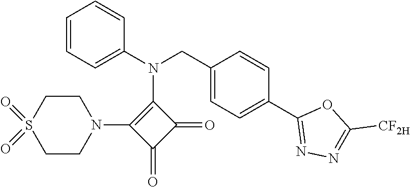 [SMILES: O=c1c(N2CCS(=O)(=O)CC2)c(N(Cc2ccc(-c3nnc(C(F)F)o3)cc2)c2ccccc2)c1=O] |
| 6 | 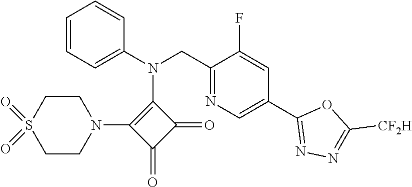 [SMILES: O=c1c(N2CCS(=O)(=O)CC2)c(N(Cc2ncc(-c3nnc(C(F)F)o3)cc2F)c2ccccc2)c1=O] |
| 7 | 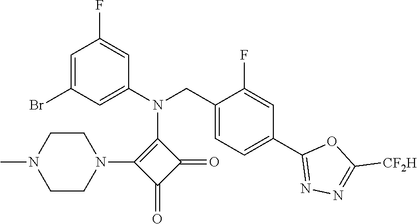 [SMILES: CN1CCN(c2c(N(Cc3ccc(-c4nnc(C(F)F)o4)cc3F)c3cc(F)cc(Br)c3)c(=O)c2=O)CC1] |
| 8 | 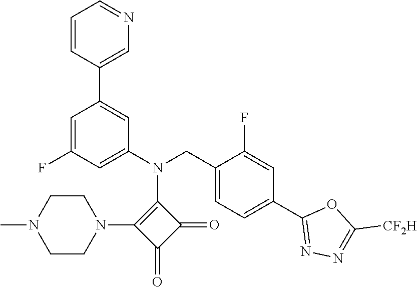 [SMILES: CN1CCN(c2c(N(Cc3ccc(-c4nnc(C(F)F)o4)cc3F)c3cc(F)cc(-c4cccnc4)c3)c(=O)c2=O)CC1] |
| 9 | 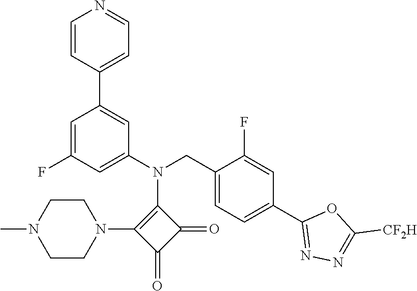 [SMILES: CN1CCN(c2c(N(Cc3ccc(-c4nnc(C(F)F)o4)cc3F)c3cc(F)cc(-c4ccncc4)c3)c(=O)c2=O)CC1] |
| 10 | 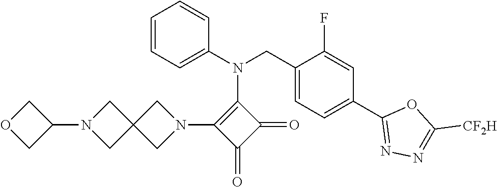 [SMILES: O=c1c(N2CC3(C2)CN(C2COC2)C3)c(N(Cc2ccc(-c3nnc(C(F)F)o3)cc2F)c2ccccc2)c1=O] |
| 11 | 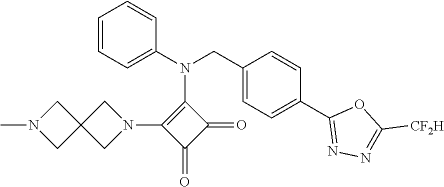 [SMILES: CN1CC2(C1)CN(c1c(N(Cc3ccc(-c4nnc(C(F)F)o4)cc3)c3ccccc3)c(=O)c1=O)C2] |
| 12 | 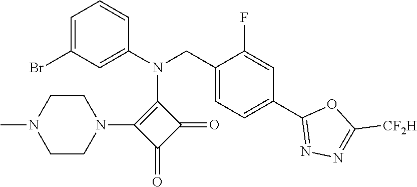 [SMILES: CN1CCN(c2c(N(Cc3ccc(-c4nnc(C(F)F)o4)cc3F)c3cccc(Br)c3)c(=O)c2=O)CC1] |
| 13 | 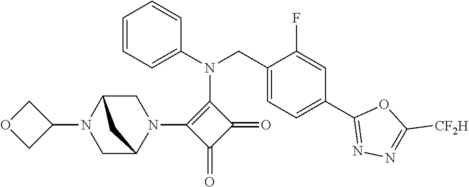 [SMILES: O=c1c(N(Cc2ccc(-c3nnc(C(F)F)o3)cc2F)c2ccccc2)c(N2C[C@@H]3C[C@H]2CN3C2COC2)c1=O] |
| 14 | 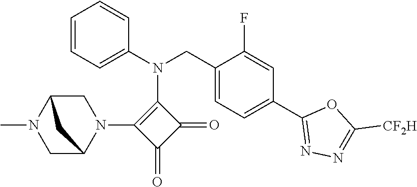 [SMILES: CN1C[C@@H]2C[C@H]1CN2c1c(N(Cc2ccc(-c3nnc(C(F)F)o3)cc2F)c2ccccc2)c(=O)c1=O] |
| 15 | 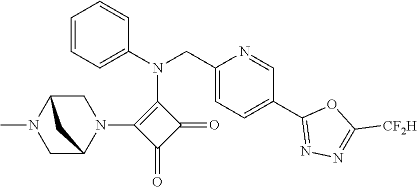 [SMILES: CN1C[C@@H]2C[C@H]1CN2c1c(N(Cc2ccc(-c3nnc(C(F)F)o3)cn2)c2ccccc2)c(=O)c1=O] |
| 16 | 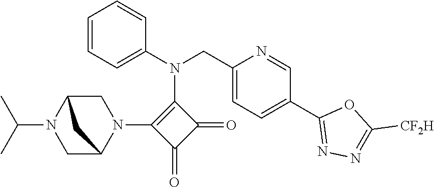 [SMILES: CC(C)N1C[C@@H]2C[C@H]1CN2c1c(N(Cc2ccc(-c3nnc(C(F)F)o3)cn2)c2ccccc2)c(=O)c1=O] |
| 17 | 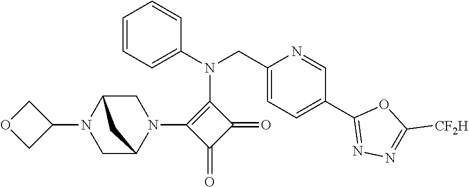 [SMILES: O=c1c(N(Cc2ccc(-c3nnc(C(F)F)o3)cn2)c2ccccc2)c(N2C[C@@H]3C[C@H]2CN3C2COC2)c1=O] |
| 18 | 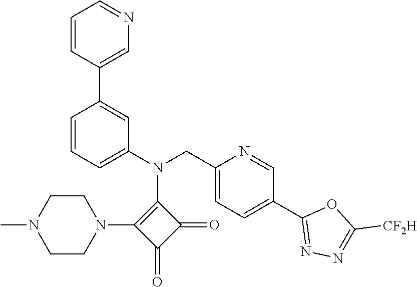 [SMILES: CN1CCN(c2c(N(Cc3ccc(-c4nnc(C(F)F)o4)cn3)c3cccc(-c4cccnc4)c3)c(=O)c2=O)CC1] |
| 19 | 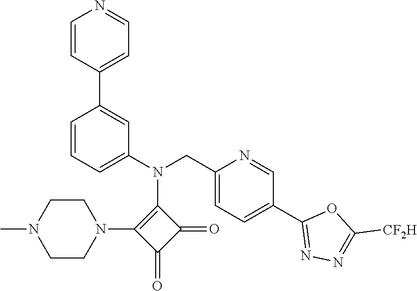 [SMILES: CN1CCN(c2c(N(Cc3ccc(-c4nnc(C(F)F)o4)cn3)c3cccc(-c4ccncc4)c3)c(=O)c2=O)CC1] |
| 20 | 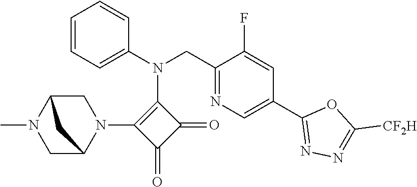 [SMILES: CN1C[C@@H]2C[C@H]1CN2c1c(N(Cc2ncc(-c3nnc(C(F)F)o3)cc2F)c2ccccc2)c(=O)c1=O] |
| 21 | 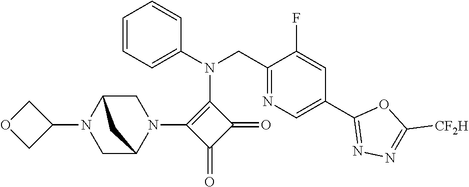 [SMILES: O=c1c(N(Cc2ncc(-c3nnc(C(F)F)o3)cc2F)c2ccccc2)c(N2C[C@@H]3C[C@H]2CN3C2COC2)c1=O] |
| 22 | 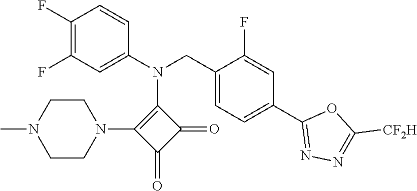 [SMILES: CN1CCN(c2c(N(Cc3ccc(-c4nnc(C(F)F)o4)cc3F)c3ccc(F)c(F)c3)c(=O)c2=O)CC1] |
| 23 | 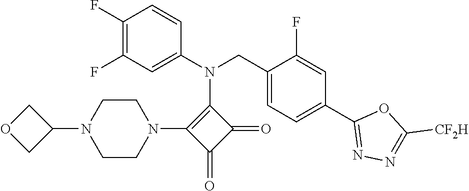 [SMILES: O=c1c(N2CCN(C3COC3)CC2)c(N(Cc2ccc(-c3nnc(C(F)F)o3)cc2F)c2ccc(F)c(F)c2)c1=O] |
| 24 | 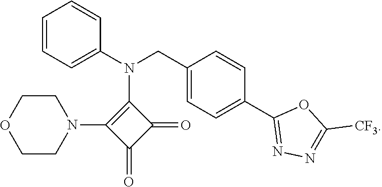 [SMILES: O=c1c(N2CCOCC2)c(N(Cc2ccc(-c3nnc(C(F)(F)F)o3)cc2)c2ccccc2)c1=O] |

7. A method of preventing or treating histone deacetylase 6-mediated diseases, comprising administering a therapeutically effective amount of the compound, the stereoisomer thereof or the pharmaceutically acceptable salt thereof of claim 1 as an active ingredient to a subject in need.

8. The method of claim 7, wherein
the histone deacetylase 6-mediated diseases are infectious diseases; neoplasm; endocrine, nutritional, and metabolic diseases; mental and behavioral disorders; neurological diseases; eye and ocular adnexal disease; circulatory diseases; respiratory diseases; digestive diseases; skin and subcutaneous tissue diseases; musculoskeletal and connective tissue diseases; or congenital malformations, alterations or chromosomal abnormalities.

9. (canceled)

10. (canceled)

11. A method of preventing or treating histone deacetylase 6-mediated diseases, comprising administering a therapeutically effective amount of the compound, the stereoisomer thereof or the pharmaceutically acceptable salt thereof claim 2 as an active ingredient to a subject in need.

12. A method of preventing or treating histone deacetylase 6-mediated diseases, comprising administering a therapeutically effective amount of the compound, the stereoisomer thereof or the pharmaceutically acceptable salt thereof claim 3 as an active ingredient to a subject in need.

13. A method of preventing or treating histone deacetylase 6-mediated diseases, comprising administering a therapeutically effective amount of the compound, the stereoisomer thereof or the pharmaceutically acceptable salt thereof claim 4 as an active ingredient to a subject in need.

14. A method of preventing or treating histone deacetylase 6-mediated diseases, comprising administering a therapeutically effective amount of the compound, the stereoisomer thereof or the pharmaceutically acceptable salt thereof claim 5 as an active ingredient to a subject in need.

15. A method of preventing or treating histone deacetylase 6-mediated diseases, comprising administering a therapeutically effective amount of the compound, the stereoisomer thereof or the pharmaceutically acceptable salt thereof claim 6 as an active ingredient to a subject in need.

16. A composition comprising the 1,3,4-oxadiazole derivative compound of claim 1 and a pharmaceutically acceptable carrier.

17. A method of preventing or treating histone deacetylase 6-mediated diseases, comprising administering the composition of claim 16 to a subject in need.
